(19) United States
(12) Patent Application Publication
(10) Pub. No.: US 2022/0048893 A1
(43) Pub. Date: Feb. 17, 2022

(54) Compounds for Treating Neurodegenerative Diseases and Cancers

(71) Applicant: Hongyi & Associates LLC, Princeton, NJ (US)

(72) Inventor: Weilin Sun, Princeton, NJ (US)

(21) Appl. No.: 17/435,803

(22) PCT Filed: Mar. 5, 2020
(86) PCT No.: PCT/US2020/021063

§ 371 Date: Sep. 2, 2021

Related U.S. Application Data
(60) Provisional application No. 62/814,160, filed on Mar. 5, 2019.

(51) Int. Cl. C07D 401/14; C07D 401/04

(52) U.S. Cl. CPC C07D 401/14; C07D 401/04

(57) ABSTRACT

The present invention relates to novel substituted aromatic-aliphatic amines capable of penetrating blood brain barrier and mediating pathogenic process in neurodegenerative diseases.

### CROSS-REFERENCE TO RELATED APPLICATIONS

This application claims priority to U.S. Provisional Patent Application No. 62/814,160, filed on Mar. 5, 2019, which is hereby incorporated by reference in its entirety.

### FIELD OF INVENTION

The present invention relates to novel substituted aromatic-aliphatic amines capable of penetrating blood brain barrier and mediating pathogenic process in neurodegenerative diseases, such as Alzheimer's Disease (AD), Parkinson's Disease (PD), amyotrophic lateral sclerosis (ALS) and Huntington's disease (HD). Further, the compounds of the present invention inhibit certain kinases, thereby being useful for treating cancers.

### BACKGROUND OF INVENTION

Neurodegenerative diseases are a group of heterogeneous disorders with features of gradually progressive loss of neural cells and neurons, eventually leading to compromised motor and/or cognitive functions. The etiology of neurodegenerative diseases remains largely unknown.

The hallmarks of AD, the most prevalent neurodegenerative disease, are characterized by progressive neuronal loss, accumulation of extracellular amyloid plaque and intracellular neurofibrillary tangle and neuronal inflammation.

PD is the second most common neurodegenerative disease. Degeneration of dopaminergic neuron in the sub stantia nigra pars compacta and the loss of DA levels in the nigrostriatal DA pathway in the brain are the main features of PD. The molecular mechanism of PD remains elusive. Aggregation of the toxic misfolded α-synuclein and subsequent formation of Lewy body play roles in the pathological progression of PD. Although etiology of AD and PD is different, these two neurodegenerative disorders share the common pathological events including protein misfolding and accumulation of aggregates.

Given the devastating situation to the neurodegenerative disease patients and their families, new treatments or therapeutics are of significant unmet medical need.

An Abl kinase inhibitor Imatinib was found to inhibit production of Amyloid-β (Aβ) peptides without affecting γ-secretase cleavage of Notch and show no neuron toxicity (Netzer, PNAS 2003). But Imatinib does not pass through blood brain barrier probably due to it as a p-glycoprotein substrate. The second generation Abl kinase inhibitor Nilotinib showed improved brain penetration and reduction of amyloid and p-tau in preclinical models (Hebron, et al. Tyrosine Kinase Inhibition Regulates Early Systemic Immune Changes and Modulates the Neuroimmune Response in α-Synucleinopathy. J Clin Cell Immunol, 5, 259; Lonskaya, et al. Nilotinib-induced autophagic changes increase endogenous parkin level and ubiquitination, leading to amyloid clearance. J Mol Med, 92, 373-386). Nilotinib was also reported to show in vivo efficacy and prevent dopaminergic neuron loss and behavioral deficits in a preclinical PD animal model (Senthilkumar et al. The c-Abl inhibitor, Nilotinib, protects dopaminergic neurons in a preclinical animal model of Parkinson's disease. Scientific Reports 2014, 1-8). In a small non-controlled pilot clinical trial for the treatment of PD, Nilotinib may show some minor beneficial effects on motor and cognitive performance for PD patients after 6 month treatment (Pagan, et al. Nilotinib Effects in Parkinson's disease and Dementia with Lewy Bodies. Journal of Parkinson's Diseases. 6 (2016) 503-517). From two recently completed PD clinical trials, Nilotinib showed effects on some biomarkers but fail to improve motor mobility in PD patients in the trial, probably due to its limit brain permeability and mild potency (Pagan et al. Nilotinib effects on safety, tolerability, and potential biomarkers in Parkinson Disease. JAMA Neurology. 2019. doi:10.1001/jamaneuro1.2019.4200). Tasigna®, brand name of Nilotinib, has a black box waring for it is associated with potentially severe cardiac side effects. Ponatinib, an approved kinase inhibitor for chronic myeloid leukemia (AML) and acute lymphoblatic leukemia (ALL) with black box warning, is also intended for treating or preventing neurodegenerative diseases (WO 2012/139029 A1). Therefore, there is growing need to develop more brain permeable, and safer novel compounds for neurodegenerative diseases (Brahmachari, et al. c-Abl and Parkinson's Disease: Mechanisms and Therapeutic Potential. Journal of Parkinson's Disease 7 (2017) 589-601).

### SUMMARY OF THE INVENTION

The present invention provides compounds of Formula (I) or a pharmaceutically acceptable salt, solvate, hydrate, cocrystal, or prodrug thereof. The compounds of Formula (I) may be present as a single stereoisomer (e.g., enriched to at least 95% purity relative to the total amount of all stereoisomers present), a racemate, or a mixture of enantiomers or diastereomers in any ratio.

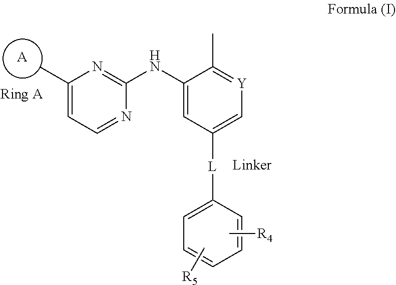

wherein Ring A is a substituted or unsubstituted 6-membered heteroaryl ring containing 1 or 2 nitrogen atoms with the remaining ring atoms being carbon;
L is a linker is selected from the group consisting of —NHC(O)—, —C(O)NH—, —SO₂NH—, —NHSO₂—, —C(CF₃)NH—, —NH—C(CF₃)—, —C(CH₂CH₂)—NH—, —NH(CH₂CH₂)C—, —C(F)═CH—, —CH═(F)C—, —C(CH₂OCH₂)NH—, —NH(CH₂OCH₂)C—, —C(CH₂OCH₂)O—, —O(CH₂OCH₂)C—, and combinations thereof;
Y═CH, or N;
R₄ is selected from the group consisting of hydrogen, halogens, —OMe, —CF₃, cyano, and ethylene; and
R₅ is an unsubstituted or substituted alkylamine.

This invention relates to novel compounds that cross blood brain barrier, inhibit the formation and accumulation of AP as well as the hyperphosphorylation of tau, prevent dopaminergic neuron loss and behavioral deficits, reduce the accumulation of α-synuclein and formation of Lewy body; and are useful for the treatment of neurodegenerative diseases particularly PD and AD.

### BRIEF DESCRIPTION OF THE DRAWINGS

The foregoing summary, as well as the following detailed description of the invention, will be better understood when read in conjunction with the appended drawings.

FIG. 1 illustrates Imatinib Analog 1's potency on reducing Aβ40 and Aβ42 levels compared to Imatinib.

FIG. 2 illustrates the structure of Imatinib Analog 1.

### DETAILED DESCRIPTION OF THE INVENTION

### Definitions

The term “H” denotes a single hydrogen atom. This radical may be attached, for example, to an oxygen atom to form a hydroxyl radical.

Where the term “alkyl” is used, either alone or within other terms such as “haloalkyl” or “alkylamino”, it embraces linear or branched radicals having one to about twelve carbon atoms. More preferred alkyl radicals are “lower alkyl” radicals having one to about six carbon atoms. Examples of such radicals include methyl, ethyl, n-propyl, isopropyl, n-butyl, isobutyl, sec-butyl, tert-butyl, pentyl, isoamyl, hexyl and the like. Even more preferred are lower alkyl radicals having one or two carbon atoms. The term “alkylenyl” or “alkylene” embraces bridging divalent alkyl radicals such as methylenyl or ethylenyl. The term “lower alkyl substituted with R²” does not include an acetal moiety. The term “alkyl” further includes alkyl radicals wherein one or more carbon atoms in the chain is substituted with a heteroatom selected from oxygen, nitrogen, or sulfur.

The term “alkenyl” embraces linear or branched radicals having at least one carbon-carbon double bond of two to about twelve carbon atoms. More preferred alkenyl radicals are “lower alkenyl” radicals having two to about six carbon atoms. Most preferred lower alkenyl radicals are radicals having two to about four carbon atoms. Examples of alkenyl radicals include ethenyl, propenyl, allyl, propenyl, butenyl and 4-methylbutenyl. The terms “alkenyl” and “lower alkenyl”, embrace radicals having “cis” and “trans” orientations, or alternatively, “E” and “Z” orientations.

The term “alkynyl” denotes linear or branched radicals having at least one carbon-carbon triple bond and having two to about twelve carbon atoms. More preferred alkynyl radicals are “lower alkynyl” radicals having two to about six carbon atoms. Most preferred are lower alkynyl radicals having two to about four carbon atoms. Examples of such radicals include propargyl, and butynyl, and the like.

Alkyl, alkylenyl, alkenyl, and alkynyl radicals may be optionally substituted with one or more functional groups such as halo, hydroxy, nitro, amino, cyano, haloalkyl, aryl, heteroaryl, and heterocyclo and the like.

The term “halo” means halogens such as fluorine, chlorine, bromine or iodine atoms.

The term “haloalkyl” embraces radicals wherein any one or more of the alkyl carbon atoms is substituted with halo as defined above. Specifically embraced are monohaloalkyl, dihaloalkyl and polyhaloalkyl radicals including perhaloalkyl. A monohaloalkyl radical, for example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having 1 to 6 carbon atoms. Even more preferred are lower haloalkyl radicals having one to three carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “perfluoroalkyl” means alkyl radicals having all hydrogen atoms replaced with fluoro atoms. Examples include trifluoromethyl and pentafluoroethyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl. Even more preferred are lower hydroxyalkyl radicals having one to three carbon atoms.

The term “alkoxy” embraces linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. Even more preferred are lower alkoxy radicals having one to three carbon atoms. Alkoxy radicals may be further substituted with one or more halo atoms, such as fluoro, chloro or bromo, to provide “haloalkoxy” radicals. Even more preferred are lower haloalkoxy radicals having one to three carbon atoms. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one or two rings, wherein such rings may be attached together in a fused manner. The term “aryl” embraces aromatic radicals such as phenyl, naphthyl, indenyl, tetrahydronaphthyl, and indanyl. More preferred aryl is phenyl. An “aryl” group may have 1 or more substituents such as lower alkyl, hydroxyl, halo, haloalkyl, nitro, cyano, alkoxy, and lower alkylamino, and the like. Phenyl substituted with —O—CH₂—O— forms the aryl benzodioxolyl substituent.

The term “heterocyclyl” (or “heterocyclo”) embraces saturated, partially saturated and unsaturated heteroatom-containing ring radicals, where the heteroatoms may be selected from nitrogen, sulfur and oxygen. It does not include rings containing —O—O—, —O—S— or —S—S— portions. The “heterocyclyl” group may have 1 to 4 substituents such as hydroxyl, Boc, halo, haloalkyl, cyano, lower alkyl, lower aralkyl, oxo, lower alkoxy, amino and lower alkylamino.

Examples of saturated heterocyclic radicals include saturated 3 to 6-membered heteromonocyclic groups containing 1 to 4 nitrogen atoms [e.g., pyrrolidinyl, imidazolidinyl, piperidinyl, pyrrolinyl, piperazinyl]; saturated 3 to 6-membered heteromonocyclic group containing 1 to 2 oxygen atoms and 1 to 3 nitrogen atoms [e.g., morpholinyl]; saturated 3 to 6-membered heteromonocyclic group containing 1 to 2 sulfur atoms and 1 to 3 nitrogen atoms [e.g., thiazolidinyl]. Examples of partially saturated heterocyclyl radicals include dihydrothienyl, dihydropyranyl, dihydrofuryl and dihydrothiazolyl.

Examples of unsaturated heterocyclic radicals, also termed “heteroaryl” radicals, include unsaturated 5 to 6 membered heteromonocyclyl group containing 1 to 4 nitrogen atoms, for example, pyrrolyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl]; unsaturated 5- to 6-membered heteromonocyclic group containing an oxygen atom, for example, pyranyl, 2-furyl, 3-furyl, etc.; unsaturated 5 to 6-membered heteromonocyclic group containing a sulfur atom, for example, 2-thienyl, 3-thienyl, etc.; unsaturated 5- to 6-membered heteromonocyclic group containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic group containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl].

The term heterocyclyl, (or heterocyclo) also embraces radicals where heterocyclic radicals are fused/condensed with aryl radicals: unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo [1,5-b]pyridazinyl]; unsaturated condensed heterocyclic group containing 1 to 2 oxygen atoms and 1 to 3 nitrogen atoms [e.g. benzoxazolyl, benzoxadiazolyl]; unsaturated condensed heterocyclic group containing 1 to 2 sulfur atoms and 1 to 3 nitrogen atoms [e.g., benzothiazolyl, benzothiadiazolyl]; and saturated, partially unsaturated and unsaturated condensed heterocyclic group containing 1 to 2 oxygen or sulfur atoms [e.g. benzofuryl, benzothienyl, 2,3-dihydro-benzo[1,4]dioxinyl and dihydrobenzofuryl]. Preferred heterocyclic radicals include five to ten membered fused or unfused radicals. More preferred examples of heteroaryl radicals include quinolyl, isoquinolyl, imidazolyl, pyridyl, thienyl, thiazolyl, oxazolyl, furyl and pyrazinyl. Other preferred heteroaryl radicals are 5- or 6-membered heteroaryl, containing one or two heteroatoms selected from sulfur, nitrogen and oxygen, selected from thienyl, furyl, pyrrolyl, indazolyl, pyrazolyl, oxazolyl, triazolyl, imidazolyl, pyrazolyl, isoxazolyl, isothiazolyl, pyridyl, piperidinyl and pyrazinyl.

Particular examples of non-nitrogen containing heteroaryl include pyranyl, 2-furyl, 3-furyl, 2-thienyl, 3-thienyl, benzofuryl, and benzothienyl, and the like.

Particular examples of partially saturated and saturated heterocyclyl include pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl, and the like.

The term “heterocyclo” thus encompasses the following ring systems:

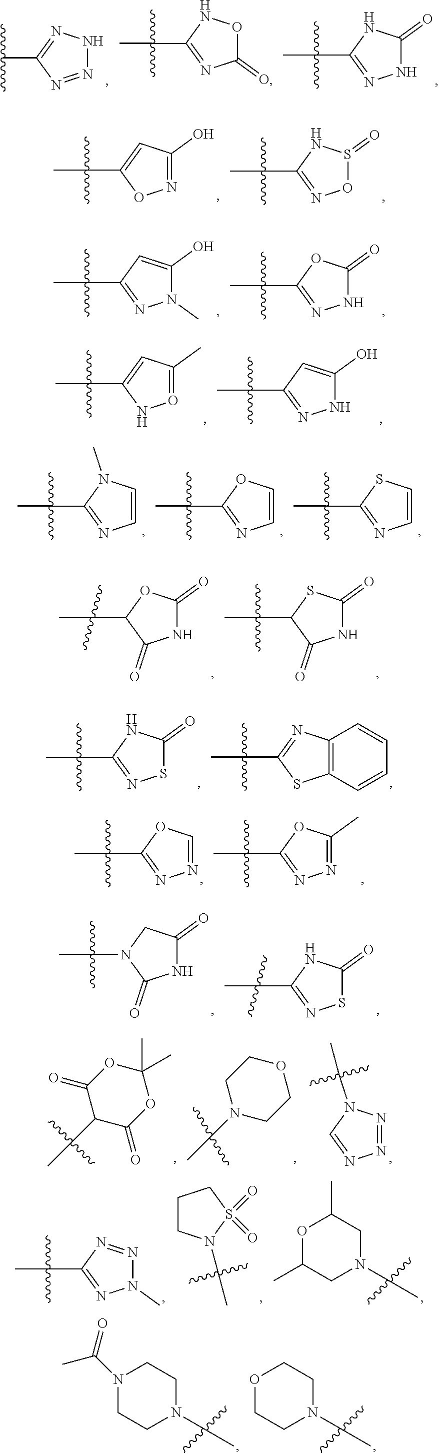

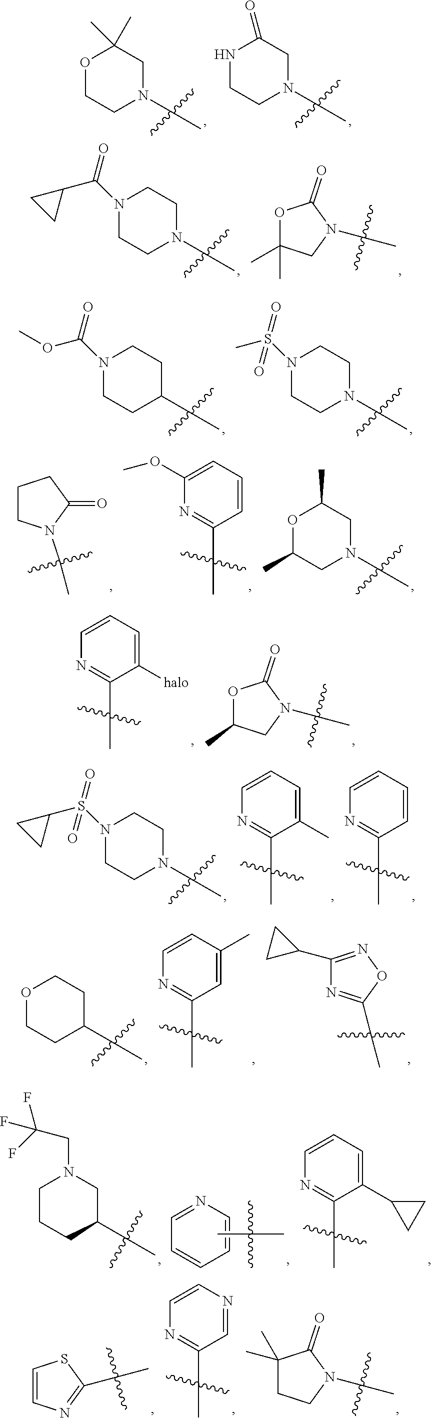

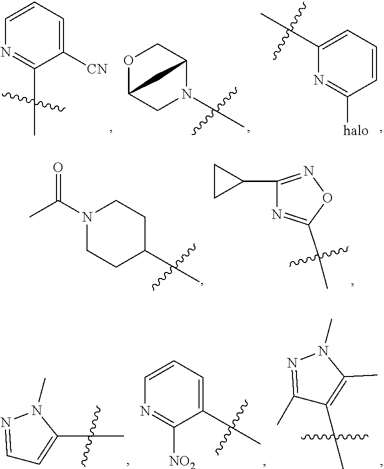

and the like.

The term “sulfonyl”, whether used alone or linked to other terms such as alkylsulfonyl, denotes respectively divalent radicals —SO₂—.

The terms “sulfamyl,” “aminosulfonyl” and “sulfonamidyl,” denotes a sulfonyl radical substituted with an amine radical, forming a sulfonamide (—SO₂NH₂).

The term “alkylaminosulfonyl” includes “N-alkylaminosulfonyl” where sulfamyl radicals are independently substituted with one or two alkyl radical(s). More preferred alkylaminosulfonyl radicals are “lower alkylaminosulfonyl” radicals having one to six carbon atoms. Even more preferred are lower alkylaminosulfonyl radicals having one to three carbon atoms. Examples of such lower alkylaminosulfonyl radicals include N-methylaminosulfonyl, and N-ethylaminosulfonyl.

The terms “carboxy” or “carboxyl,” whether used alone or with other terms, such as “carboxyalkyl,” denotes —CO₂H.

The term “carbonyl,” whether used alone or with other terms, such as “aminocarbonyl,” denotes —(C═O)—.

The term “aminocarbonyl” denotes an amide group of the formula C(═O)NH₂.

The terms “N-alkylaminocarbonyl” and “N,N-dialkylaminocarbonyl” denote aminocarbonyl radicals independently substituted with one or two alkyl radicals, respectively. More preferred are “lower alkylaminocarbonyl” having lower alkyl radicals as described above attached to an aminocarbonyl radical.

The terms “N-arylaminocarbonyl” and “N-alkyl-N-arylaminocarbonyl” denote aminocarbonyl radicals substituted, respectively, with one aryl radical, or one alkyl and one aryl radical.

The terms “heterocyclylalkylenyl” and “heterocyclylalkyl” embrace heterocyclic-substituted alkyl radicals. More preferred heterocyclylalkyl radicals are “5- or 6-membered heteroarylalkyl” radicals having alkyl portions of one to six carbon atoms and a 5- or 6-membered heteroaryl radical. Even more preferred are lower heteroarylalkylenyl radicals having alkyl portions of one to three carbon atoms. Examples include such radicals as pyridylmethyl and thienylmethyl.

The term “aralkyl” embraces aryl-substituted alkyl radicals. Preferable aralkyl radicals are “lower aralkyl” radicals having aryl radicals attached to alkyl radicals having one to six carbon atoms. Even more preferred are “phenylalkylenyl” attached to alkyl portions having one to three carbon atoms. Examples of such radicals include benzyl, diphenylmethyl and phenylethyl. The aryl in said aralkyl may be additionally substituted with halo, alkyl, alkoxy, halkoalkyl and haloalkoxy.

The term “alkylthio” embraces radicals containing a linear or branched alkyl radical, of one to ten carbon atoms, attached to a divalent sulfur atom. Even more preferred are lower alkylthio radicals having one to three carbon atoms. An example of “alkylthio” is methylthio, (CH₃S—).

The term “haloalkylthio” embraces radicals containing a haloalkyl radical, of one to ten carbon atoms, attached to a divalent sulfur atom. Even more preferred are lower haloalkylthio radicals having one to three carbon atoms. An example of “haloalkylthio” is trifluoromethylthio.

The term “alkylamino” embraces “N-alkylamino” and “N,N-dialkylamino” where amino groups are independently substituted with one alkyl radical and with two alkyl radicals, respectively. More preferred alkylamino radicals are “lower alkylamino” radicals having one or two alkyl radicals of one to six carbon atoms, attached to a nitrogen atom. Even more preferred are lower alkylamino radicals having one to three carbon atoms. Suitable alkylamino radicals may be mono or di(C1-C6)alkylamino such as N-methylamino, N-ethylamino, N,N-dimethylamino, and N,N-diethylamino, and the like.

The term “arylamino” denotes amino groups, which have been substituted with one or two aryl radicals, such as N-phenylamino. The arylamino radicals may be further substituted on the aryl ring portion of the radical.

The term “heteroarylamino” denotes amino groups, which have been substituted with one or two heteroaryl radicals, such as N-thienylamino. The “heteroarylamino” radicals may be further substituted on the heteroaryl ring portion of the radical.

The term “aralkylamino” denotes amino groups, which have been substituted with one or two aralkyl radicals. More preferred are phenyl-C₁-C₃-alkylamino radicals, such as N-benzylamino. The aralkylamino radicals may be further substituted on the aryl ring portion.

The terms “N-alkyl-N-arylamino” and “N-aralkyl-N-alkylamino” denote amino groups, which have been independently substituted with one aralkyl and one alkyl radical, or one aryl and one alkyl radical, respectively, to an amino group.

The term “aminoalkyl” embraces linear or branched alkyl radicals having one to about ten carbon atoms any one of which may be substituted with one or more amino radicals. More preferred aminoalkyl radicals are “lower aminoalkyl” radicals having one to six carbon atoms and one or more amino radicals. Examples of such radicals include aminomethyl, aminoethyl, aminopropyl, aminobutyl and aminohexyl. Even more preferred are lower aminoalkyl radicals having one to three carbon atoms.

The term “alkylaminoalkyl” embraces alkyl radicals substituted with alkylamino radicals. More preferred alkylaminoalkyl radicals are “lower alkylaminoalkyl” radicals having alkyl radicals of one to six carbon atoms. Even more preferred are lower alkylaminoalkyl radicals having alkyl radicals of one to three carbon atoms. Suitable alkylaminoalkyl radicals may be mono or dialkyl substituted, such as N-methylaminomethyl, N,N-dimethyl-aminoethyl, and N,N-diethylaminomethyl, and the like.

The term “alkylaminoalkoxy” embraces alkoxy radicals substituted with alkylamino radicals. More preferred alkylaminoalkoxy radicals are “lower alkylaminoalkoxy” radicals having alkoxy radicals of one to six carbon atoms. Even more preferred are lower alkylaminoalkoxy radicals having alkyl radicals of one to three carbon atoms. Suitable alkylaminoalkoxy radicals may be mono or dialkyl substituted, such as N-methylaminoethoxy, N,N-dimethylaminoethoxy, and N,N-diethylaminoethoxy, and the like.

The term “alkylaminoalkoxyalkoxy” embraces alkoxy radicals substituted with alkylaminoalkoxy radicals. More preferred alkylaminoalkoxyalkoxy radicals are “lower alkylaminoalkoxyalkoxy” radicals having alkoxy radicals of one to six carbon atoms. Even more preferred are lower alkylaminoalkoxyalkoxy radicals having alkyl radicals of one to three carbon atoms. Suitable alkylaminoalkoxyalkoxy radicals may be mono or dialkyl substituted, such as N-methylaminomethoxyethoxy, N-methylaminoethoxyethoxy, N,N-dimethylaminoethoxyethoxy, and N,N-diethylaminomethoxymethoxy, and the like.

The term “carboxyalkyl” embraces linear or branched alkyl radicals having one to about ten carbon atoms any one of which may be substituted with one or more carboxy radicals. More preferred carboxyalkyl radicals are “lower carboxyalkyl” radicals having one to six carbon atoms and one carboxy radical. Examples of such radicals include carboxymethyl, and carboxypropyl, and the like. Even more preferred are lower carboxyalkyl radicals having one to three CH₂ groups.

The term “halosulfonyl” embraces sulfonyl radicals substituted with a halogen radical. Examples of such halosulfonyl radicals include chlorosulfonyl and fluorosulfonyl.

The term “arylthio” embraces aryl radicals of six to ten carbon atoms, attached to a divalent sulfur atom. An example of “arylthio” is phenylthio.

The term “aralkylthio” embraces aralkyl radicals as described above, attached to a divalent sulfur atom. More preferred are phenyl-C₁-C₃-alkylthio radicals. An example of “aralkylthio” is benzylthio.

The term “aryloxy” embraces optionally substituted aryl radicals, as defined above, attached to an oxygen atom. Examples of such radicals include phenoxy.

The term “aralkoxy” embraces oxy-containing aralkyl radicals attached through an oxygen atom to other radicals. More preferred aralkoxy radicals are “lower aralkoxy” radicals having optionally substituted phenyl radicals attached to lower alkoxy radical as described above.

The term “heteroaryloxy” embraces optionally substituted heteroaryl radicals, as defined above, attached to an oxygen atom.

The term “heteroarylalkoxy” embraces oxy-containing heteroarylalkyl radicals attached through an oxygen atom to other radicals. More preferred heteroarylalkoxy radicals are “lower heteroarylalkoxy” radicals having optionally substituted heteroaryl radicals attached to lower alkoxy radical as described above.

The term “cycloalkyl” includes saturated carbocyclic groups. Preferred cycloalkyl groups include C₃-C₆ rings. More preferred compounds include, cyclopentyl, cyclopropyl, and cyclohexyl.

The term “cycloalkylalkyl” embraces cycloalkyl-substituted alkyl radicals. Preferable cycloalkylalkyl radicals are “lower cycloalkylalkyl” radicals having cycloalkyl radicals attached to alkyl radicals having one to six carbon atoms. Even more preferred are “5 to 6-membered cycloalkylalkyl” attached to alkyl portions having one to three carbon atoms. Examples of such radicals include cyclohexylmethyl. The cycloalkyl in said radicals may be additionally substituted with halo, alkyl, alkoxy and hydroxy.

The term “cycloalkenyl” includes carbocyclic groups having one or more carbon-carbon double bonds including “cycloalkyldienyl” compounds. Preferred cycloalkenyl groups include C₃-C₆ rings. More preferred compounds include, for example, cyclopentenyl, cyclopentadienyl, cyclohexenyl and cycloheptadienyl.

The term “comprising” is meant to be open ended, including the indicated component but not excluding other elements.

A group or atom that replaces a hydrogen atom is also called a substituent.

Any particular molecule or group can have one or more substituent depending on the number of hydrogen atoms that can be replaced.

The symbol “—” represents a covalent bond and can also be used in a radical group to indicate the point of attachment to another group. In chemical structures, the symbol is commonly used to represent a methyl group in a molecule.

The term “therapeutically effective amount” means an amount of a compound that ameliorates, attenuates or eliminates one or more symptom of a particular disease or condition, or prevents or delays the onset of one of more symptom of a particular disease or condition.

The terms “patient” and “subject” may be used interchangeably and mean animals, such as dogs, cats, cows, horses, sheep and humans. Particular patients are mammals. The term patient includes males and females.

The term “pharmaceutically acceptable” means that the referenced substance, such as a compound of Formula I, or a salt of a compound of Formula I, or a formulation containing a compound of Formula I, or a particular excipient, are suitable for administration to a patient.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

The terms “treating”, “treat” or “treatment” and the like include preventative (e.g., prophylactic) and palliative treatment.

The term “excipient” means any pharmaceutically acceptable additive, carrier, diluent, adjuvant, or other ingredient, other than the active pharmaceutical ingredient (API), which is typically included for formulation and/or administration to a patient.

The compounds of the present invention are administered to a patient in a therapeutically effective amount. The compounds can be administered alone or as part of a pharmaceutically acceptable composition or formulation. In addition, the compounds or compositions can be administered all at once, as for example, by a bolus injection, multiple times, such as by a series of tablets, or delivered substantially uniformly over a period of time, as for example, using transdermal delivery. It is also noted that the dose of the compound can be varied over time.

In addition, the compounds of the present invention can be administered alone, in combination with other compounds of the present invention, or with other pharmaceutically active compounds. The other pharmaceutically active compounds can be intended to treat the same disease or condition as the compounds of the present invention or a different disease or condition. If the patient is to receive or is receiving multiple pharmaceutically active compounds, the compounds can be administered simultaneously, or sequentially. For example, in the case of tablets, the active compounds may be found in one tablet or in separate tablets, which can be administered at once or sequentially in any order. In addition, it should be recognized that the compositions may be different forms. For example, one or more compound may be delivered via a tablet, while another is administered via injection or orally as a syrup. All combinations, delivery methods and administration sequences are contemplated.

### Compounds

The compounds of the present invention are presented by Formula (I) or a pharmaceutically acceptable salt, solvate, hydrate, cocrystal, or prodrug thereof.

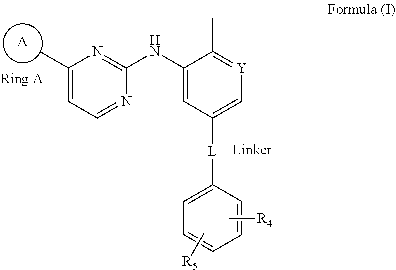

The compounds of Formula (I) may be present as a single stereoisomer (e.g., enriched to at least 95% purity relative to the total amount of all stereoisomers present), a racemate, or a mixture of enantiomers or diastereomers in any ratio.

Ring A is a 6-membered heteroaryl ring containing 1 or 2 nitrogen atoms with the remaining ring atoms being carbon. Ring A is unsubstituted or substituted on the ring. The substituted group is selected from halogens, substituted and unsubstituted aryl, substituted and unsubstituted heteroaryl, and substituted and unsubstituted heterocyclyl.

In certain embodiments, Ring A is selected from:

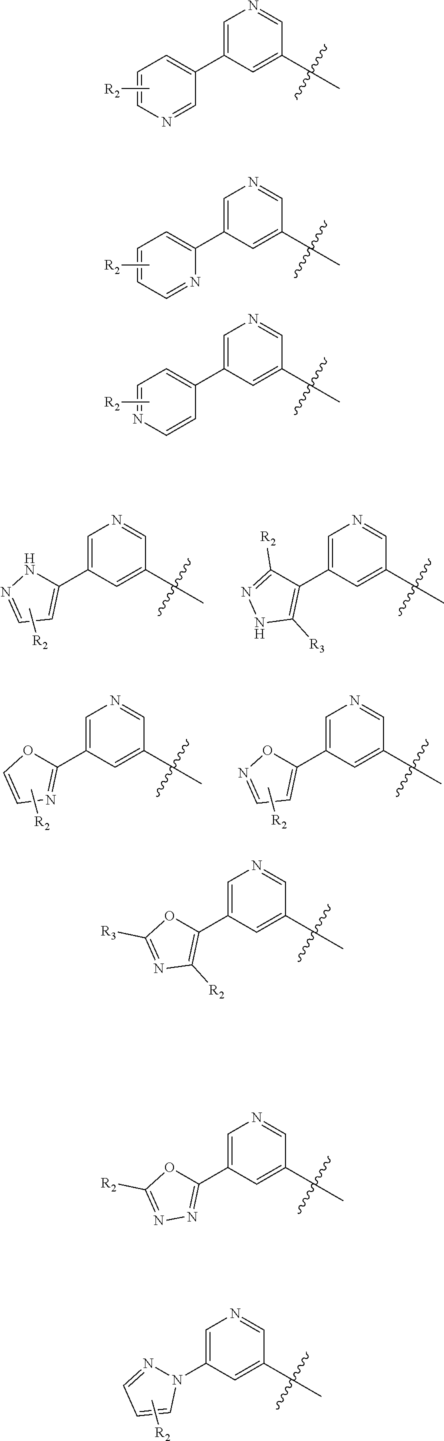

wherein R₂ and R₃ independently for each occurrence are selected from the group consisting of hydrogen, halogen, (C1-C6)alkyl, amino, cyano, (C2-C8)alkynyl, mono(C1-C6)alkylamino, di(C1-C6)alkyl amino, and (C₁-C₆)alkyoxy;

L is a linker is selected from the group consisting of —NHC(O)—, —C(O)NH—, —SO₂NH—, —NHSO₂—, —C(CF₃)NH—, —NH—C(CF₃)—, —C(CH₂CH₂)—NH—, —NH(CH₂CH₂)C—, —C(F)═CH—, —CH═(F)C—, —C(CH₂OCH₂)NH—, —NH(CH₂OCH₂)C—, —C(CH₂OCH₂)O—, —O(CH₂OCH₂)C—, and combinations thereof;

Y═CH, or N;

R₄ is selected from the group consisting of hydrogen, halogens, —OMe, —CF₃, cyano, and ethylene;

R₅ is an unsubstituted or substituted amine. In some embodiments, R₅ is an unsubstituted or substituted alkylamine. In some embodiments, R₅ is suitably a primary aminoalkyl, a secondary or tertiary alkyl-amino-alkyl, or a nonaromatic heterocycle-aminoalkyl. Specific R₅ groups include but are not limited to: dimethylaminoethyl, diethylaminoethyl, dipropylaminoethyl, 2-(dimethylamino)propyl, 3-piperidinyl, 1-methyl-pyrrolidin-3-yl, 1-ethyl-pyrrolidin-3-yl, 1-propyl-pyrrolidin-3-yl, 1-isopropyl-pyrrolidin-3-yl, 1-iso-butyl-pyrrolidin-3-yl, t-butyl-pyrrolidin-3-yl, 1-neo-pentyl-pyrrolidin-3-yl, 1-methyl-piperidin-3-yl, 1-ethyl-piperidin-3-yl, 1-propyl-piperidin-3-yl, 1-butyl-piperidin-3-yl, 1-isopropyl-piperidin-3-yl, 1-iso-butyl-piperidin-3-yl, 1-t-butyl-piperidin-3-yl, 1-neo-pentyl-piperidin-3-yl, 1-methyl-piperidin-4-yl, 1-ethyl-piperidin-4-yl, 1-propyl-piperidin-4-yl, 1-isopropyl-piperidin-4-yl, 1-iso-butyl-piperidin-4-yl, 1-t-butyl)-piperidin-4-yl, 1-neo-pentyl-piperidin-4-yl, 4-methyl-piperazin-1-yl, 4-ethyl-piperazin-1-yl, 4-propyl-piperazin-1-yl, 4-isopropyl-piperazin-1-yl, 4-iso-butyl-piperazin-1-yl, 4-t-butyl-piperazin-1-yl, 4-neo-pentyl-piperazin-1-yl, and 1-methyl-azepan-3-yl, 1-ethyl-azepan-3-yl, 1-propyl-azepan-3-yl, 1-ethyl-azepan-3-yl, 4-(4-Methyl-piperazin-1-yl)-2-trifluoromethyl-phenyl, 4-(4-Ethyl-piperazin-1-yl)-2-trifluoromethyl-phenyl, 4-(4-Propyl-piperazin-1-yl)-2-trifluoromethyl-phenyl, 4-(4-Isopropyl-piperazin-1-yl)-2-trifluoromethyl-phenyl, 4-(4-Isobutyl-piperazin-1-yl)-2-trifluoromethyl-phenyl, 4-(4-Isobutyl-piperazin-1-yl)-2-trifluoromethyl-phenyl, 4-(4-t-Butyl-piperazin-1-yl)-2-trifluoromethyl-phenyl, 4-(4-neo-Pentyl-piperazin-1-yl)-2-trifluoromethyl-phenyl, 4-(1-Methyl-piperidin-4-yl)-2-trifluoromethyl-phenyl, 4-(1-Ethyl-piperidin-4-yl)-2-trifluoromethyl-phenyl, 4-(1-Propyl-piperidin-4-yl)-2-trifluoromethyl-phenyl, 4-(1-Isopropyl-piperidin-4-yl)-2-trifluoromethyl-phenyl, 4-(1-Isobutyl-piperidin-4-yl)-2-trifluoromethyl-phenyl, 4-(1-t-Butyl-piperidin-4-yl)-2-trifluoromethyl-phenyl, 4-(1-neo-pentyl-piperidin-4-yl)-2-trifluoromethyl-phenyl, 4-(1-Methyl-piperidin-3-yl)-2-trifluoromethyl-phenyl, 4-(1-Ethyl-piperidin-3-yl)-2-trifluoromethyl-phenyl, 4-(1-Propyl-piperidin-3-yl)-2-trifluoromethyl-phenyl, 4-(1-Isopropyl-piperidin-3-yl)-2-trifluoromethyl-phenyl, 4-(1-Isobutyl-piperidin-3-yl)-2-trifluoromethyl-phenyl, 4-(1-t-Butyl-piperidin-3-yl)-2-trifluoromethyl-phenyl, 4-(1-neo-pentyl-piperidin-3-yl)-2-trifluoromethyl-phenyl, 3-(4-Methyl-piperazin-1-yl)-5-trifluoromethyl-phenyl, 3-(4-Ethyl-piperazin-1-yl)-5-trifluoromethyl-phenyl, 3-(4-Propyl-piperazin-1-yl)-5-trifluoromethyl-phenyl, 3-(4-Isopropyl-piperazin-1-yl)-5-trifluoromethyl-phenyl, 3-(4-Isobutyl-piperazin-1-yl)-5-trifluoromethyl-phenyl, 3-(4-t-Butyl-piperazin-1-yl)-5-trifluoromethyl-phenyl, 3-(4-neo-pentyl-piperazin-1-yl)-5-trifluoromethyl-phenyl, 3-(1-Methyl-piperidin-4-yl)-5-trifluoromethyl-phenyl, 3-(1-Ethyl-piperidin-4-yl)-5-trifluoromethyl-phenyl, 3-(1-Propyl-piperidin-4-yl)-5-trifluoromethyl-phenyl, 3-(1-Isopropyl-piperidin-4-yl)-5-trifluoromethyl-phenyl, 3-(1-Isobutyl-piperidin-4-yl)-5-trifluoromethyl-phenyl, 3-(1-t-Butyl-piperidin-4-yl)-5-trifluoromethyl-phenyl, 3-(1-neo-pentyl-piperidin-4-yl)-5-trifluoromethyl-phenyl, 3-(1-Methyl-piperidin-3-yl)-5-trifluoromethyl-phenyl, 3-(1-Ethyl-piperidin-3-yl)-5-trifluoromethyl-phenyl, 3-(1-Propyl-piperidin-3-yl)-5-trifluoromethyl-phenyl, 3-(1-Isopropyl-piperidin-3-yl)-5-trifluoromethyl-phenyl, 3-(1-Isobutyl-piperidin-3-yl)-5-trifluoromethyl-phenyl, 3-(1-t-Butyl-piperidin-3-yl)-5-trifluoromethyl-phenyl, 3-(1-neo-pentyl-piperidin-3-yl)-5-trifluoromethyl-phenyl.

In some embodiments, R₅ is a substituted alkylamine, which is masked as amide or carbamate as prodrug in an attempt to improve bioavailability and brain permeability. The masking group is selected from but are not limited to the following examples.

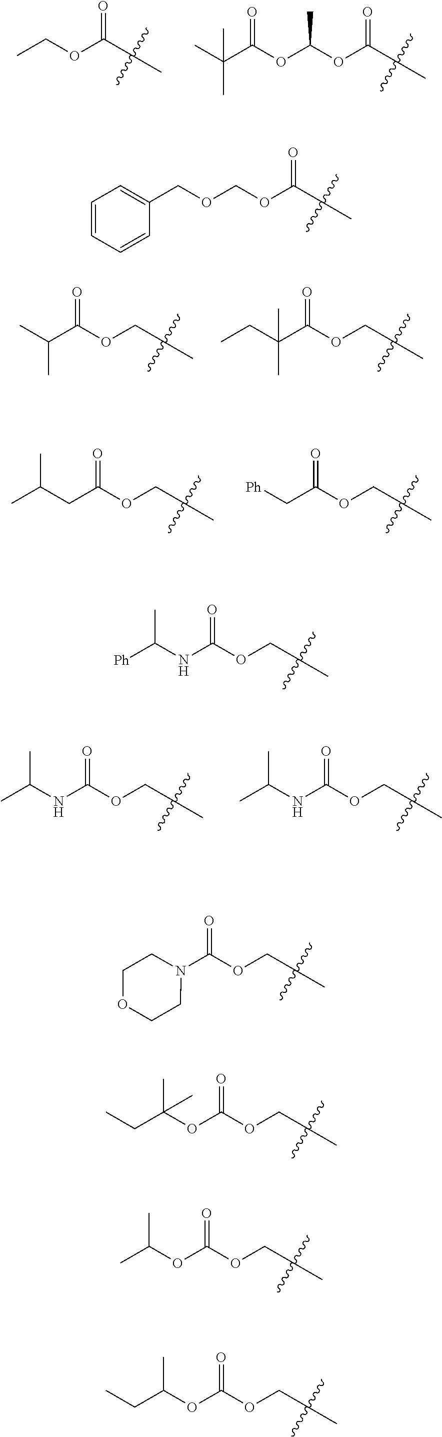

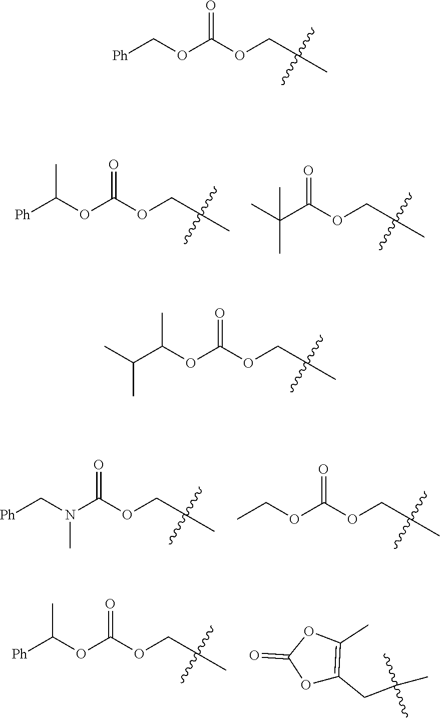

In some embodiments, the compounds of the present invention are represented in Formula (II) or a pharmaceutically acceptable salt, solvate, hydrate, cocrystal, or prodrug thereof, wherein X is CH or nitrogen; R₄ is hydrogen or CF₃; R₃ and R₅ are the same as defined in Formula (I). Examples of compounds of Formula (II) are listed in Table 1.

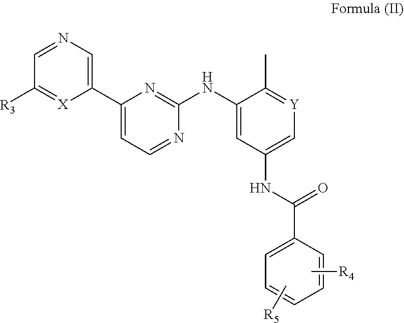

TABLE 1
Embodiments of Formula (II)

| Structure | Name |
|---|---|
| 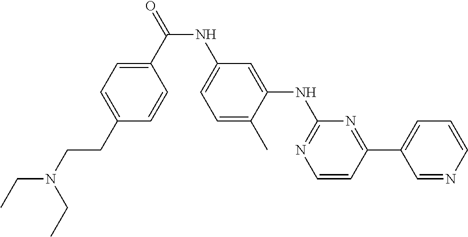 [SMILES: CCN(CC)CCc1ccc(C(=O)Nc2ccc(C)c(Nc3nccc(-c4cccnc4)n3)c2)cc1] | 4-(2-Diethylamino- ethyl)-N-[4-methyl-3-(4- pyridin-3-yl-pyrimidin- 2-ylamino)-phenyl]- benzamide |
| 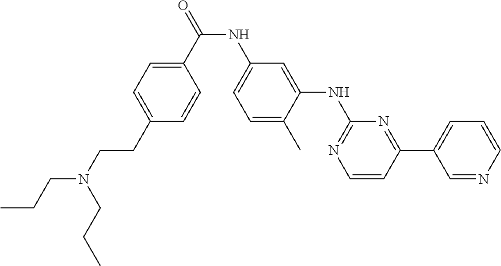 [SMILES: CCCN(CCC)CCc1ccc(C(=O)Nc2ccc(C)c(Nc3nccc(-c4cccnc4)n3)c2)cc1] | 4-(2-Dipropylamino- ethyl)-N-[4-methyl-3- (4-pyridin-3-yl-pyrimi- din-2-ylamino)-phenyl]- benzamide |
| 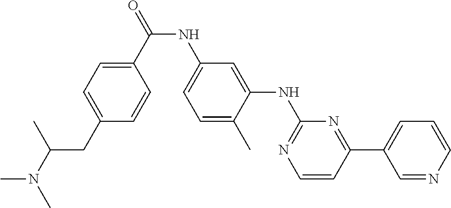 [SMILES: Cc1ccc(NC(=O)c2ccc(CC(C)N(C)C)cc2)cc1Nc1nccc(-c2cccnc2)n1] | 4-(2-Dimethylamino- propyl)-N-[4-methyl- 3-(4-pyridin-3-yl- pyrimidin-2-ylamino)- phenyl]-benzamide |
| 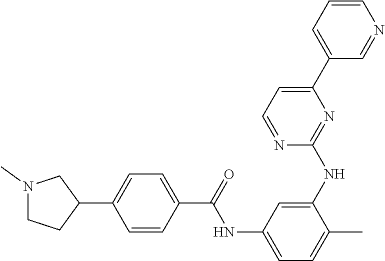 [SMILES: Cc1ccc(NC(=O)c2ccc(C3CCN(C)C3)cc2)cc1Nc1nccc(-c2cccnc2)n1] | N-[4-Methyl-3-(4- pyridin-3-yl-pyrimidin- 2-ylamino)-phenyl]-4- (1-methyl-pyrrolidin- 3-yl)-benzamide |
| 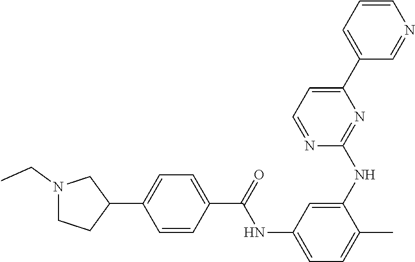 [SMILES: CCN1CCC(c2ccc(C(=O)Nc3ccc(C)c(Nc4nccc(-c5cccnc5)n4)c3)cc2)C1] | 4-(1-Ethyl-pyrrolidin- 3-yl)-N-[4-methyl-3-(4- pyridin-3-yl-pyrimidin- 2-ylamino)-phenyl]- benzamide |
| 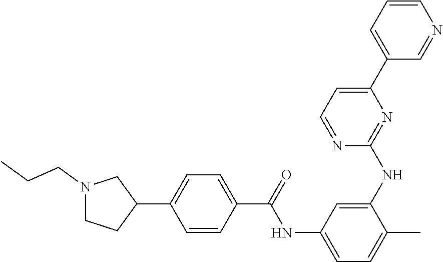 [SMILES: CCCN1CCC(c2ccc(C(=O)Nc3ccc(C)c(Nc4nccc(-c5cccnc5)n4)c3)cc2)C1] | N-[4-Methyl-3-(4- pyridin-3-yl-pyrimidin- 2-ylamino)-phenyl]-4- (1-propyl-pyrrolidin-3- yl)-benzamide |
| 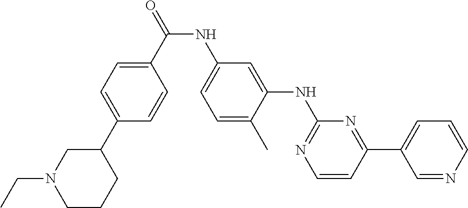 [SMILES: CCN1CCCC(c2ccc(C(=O)Nc3ccc(C)c(Nc4nccc(-c5cccnc5)n4)c3)cc2)C1] | 4-(1-Ethyl-piperidin-3- yl)-N-[4-methyl-3-(4- pyridin-3-yl-pyrimidin- 2-ylamino)-phenyl]- benzamide |
| 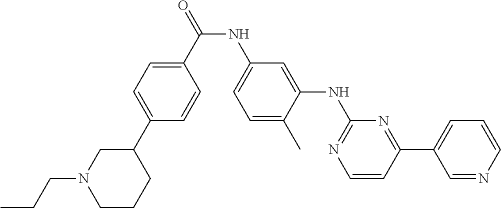 [SMILES: CCCN1CCCC(c2ccc(C(=O)Nc3ccc(C)c(Nc4nccc(-c5cccnc5)n4)c3)cc2)C1] | N-[4-Methyl-3-(4- pyridin-3-yl-pyrimidin- 2-ylamino)-phenyl]-4- (1-propyl-piperidin-3- yl)-benzamide |
| 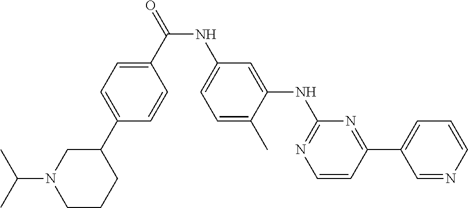 [SMILES: Cc1ccc(NC(=O)c2ccc(C3CCCN(C(C)C)C3)cc2)cc1Nc1nccc(-c2cccnc2)n1] | 4-(1-Isopropyl- piperidin-3-yl)-N-[4- methyl-3-(4-pyridin- 3-yl-pyrimidin-2- ylamino)-phenyl]- benzamide |
| 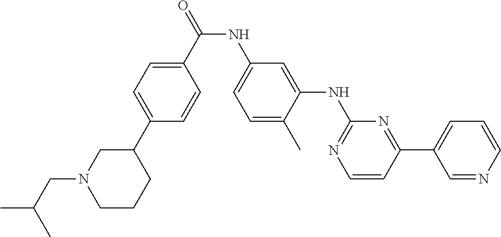 [SMILES: Cc1ccc(NC(=O)c2ccc(C3CCCN(CC(C)C)C3)cc2)cc1Nc1nccc(-c2cccnc2)n1] | 4-(1-Isobutyl-piperidin- 3-yl)-N-[4-methyl-3- (4-pyridin-3-yl-pyrimi- din-2-ylamino)-phenyl]- benzamide |
| 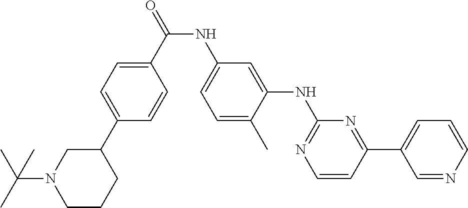 [SMILES: Cc1ccc(NC(=O)c2ccc(C3CCCN(C(C)(C)C)C3)cc2)cc1Nc1nccc(-c2cccnc2)n1] | 4-(1-tert-Butyl-piperi- din-3-yl)-N-[4-methyl- 3-(4-pyridin-3-yl- pyrimidin-2-ylamino)- phenyl]-benzamide |
| 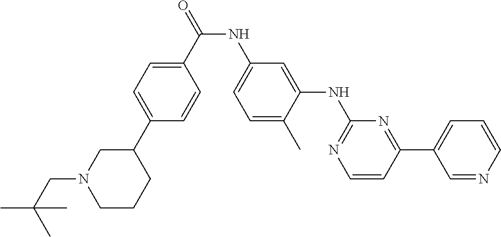 [SMILES: Cc1ccc(NC(=O)c2ccc(C3CCCN(CC(C)(C)C)C3)cc2)cc1Nc1nccc(-c2cccnc2)n1] | 4-[1-(2,2-Dimethyl- propyl)-piperidin-3-yl]- N-[4-methyl-3-(4- pyridin-3-yl-pyrimidin- 2-ylamino)-phenyl]- benzamide |
| 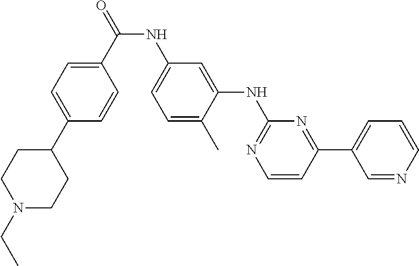 [SMILES: CCN1CCC(c2ccc(C(=O)Nc3ccc(C)c(Nc4nccc(-c5cccnc5)n4)c3)cc2)CC1] | 4-(1-Ethyl-piperidin-4- yl)-N-[4-methyl-3-(4- pyridin-3-yl-pyrimidin- 2-ylamino)-phenyl]- benzamide |
| 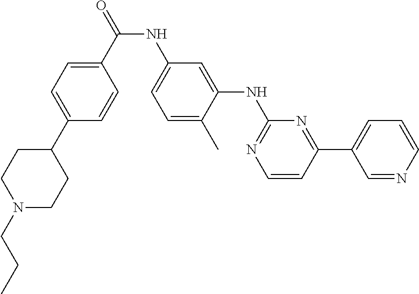 [SMILES: CCCN1CCC(c2ccc(C(=O)Nc3ccc(C)c(Nc4nccc(-c5cccnc5)n4)c3)cc2)CC1] | N-[4-Methyl-3-(4- pyridin-3-yl-pyrimidin- 2-ylamino)-phenyl]- 4-(1-propyl-piperidin- 4-yl)-benzamide |
| 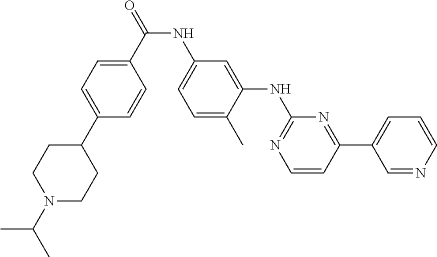 [SMILES: Cc1ccc(NC(=O)c2ccc(C3CCN(C(C)C)CC3)cc2)cc1Nc1nccc(-c2cccnc2)n1] | 4-(1-Isopropyl-piperi- din-4-yl)-N-[4-methyl- 3-(4-pyridin-3-yl- pyrimidin-2-ylamino)- phenyl]-benzamide |
| 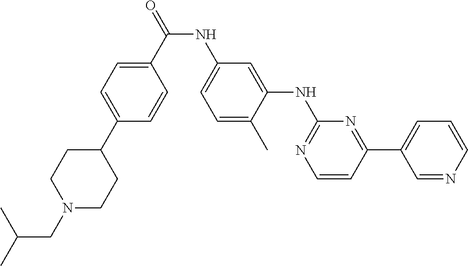 [SMILES: Cc1ccc(NC(=O)c2ccc(C3CCN(CC(C)C)CC3)cc2)cc1Nc1nccc(-c2cccnc2)n1] | 4-(1-Isobutyl-piperidin- 4-yl)-N-[4-methyl-3-(4- pyridin-3-yl-pyrimidin- 2-ylamino)-phenyl]- benzamide |
| 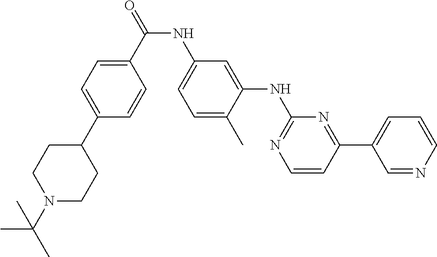 [SMILES: Cc1ccc(NC(=O)c2ccc(C3CCN(C(C)(C)C)CC3)cc2)cc1Nc1nccc(-c2cccnc2)n1] | 4-(1-tert-Butyl-piperi- din-4-yl)-N-[4-methyl- 3-(4-pyridin-3-yl- pyrimidin-2-ylamino)- phenyl]-benzamide |
| 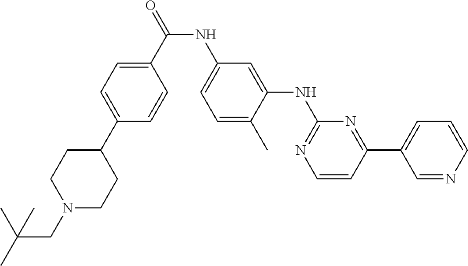 [SMILES: Cc1ccc(NC(=O)c2ccc(C3CCN(CC(C)(C)C)CC3)cc2)cc1Nc1nccc(-c2cccnc2)n1] | 4-[1-(2,2-Dimethyl- propyl)-piperidin-4-yl]- N-[4-methyl-3-(4- pyridin-3-yl-pyrimidin- 2-ylamino)-phenyl]- benzamide |
| 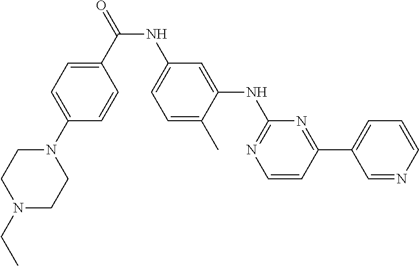 [SMILES: CCN1CCN(c2ccc(C(=O)Nc3ccc(C)c(Nc4nccc(-c5cccnc5)n4)c3)cc2)CC1] | 4-(4-Ethyl-piperazin-1- yl)-N-[4-methyl-3-(4- pyridin-3-yl-pyrimidin- 2-ylamino)-phenyl]- benzamide |
| 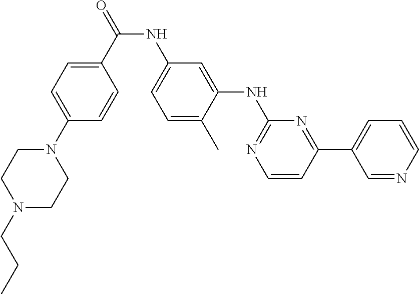 [SMILES: CCCN1CCN(c2ccc(C(=O)Nc3ccc(C)c(Nc4nccc(-c5cccnc5)n4)c3)cc2)CC1] | N-[4-Methyl-3-(4- pyridin-3-yl-pyrimidin- 2-ylamino)-phenyl]- 4-(4-propyl-piperazin- 1-yl)-benzamide |
| 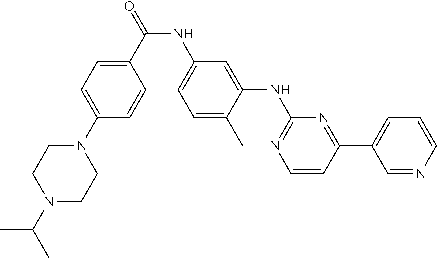 [SMILES: Cc1ccc(NC(=O)c2ccc(N3CCN(C(C)C)CC3)cc2)cc1Nc1nccc(-c2cccnc2)n1] | 4-(4-Isopropyl-pipera- zin-1-yl)-N-[4-methyl- 3-(4-pyridin-3-yl- pyrimidin-2-ylamino)- phenyl]-benzamide |
| 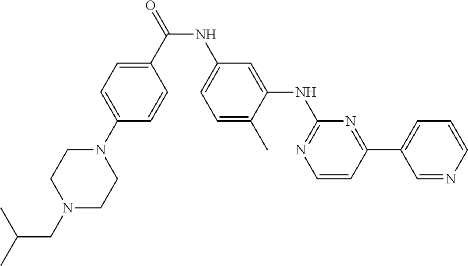 [SMILES: Cc1ccc(NC(=O)c2ccc(N3CCN(CC(C)C)CC3)cc2)cc1Nc1nccc(-c2cccnc2)n1] | 4-(4-Isobutyl-piperazin- 1-yl)-N-[4-methyl-3-(4- pyridin-3-yl-pyrimidin- 2-ylamino)-phenyl]- benzamide |
| 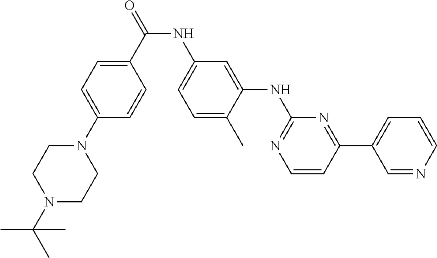 [SMILES: Cc1ccc(NC(=O)c2ccc(N3CCN(C(C)(C)C)CC3)cc2)cc1Nc1nccc(-c2cccnc2)n1] | 4-(4-tert-Butyl-pipera- zin-1-yl)-N-[4-methyl- 3-(4-pyridin-3-yl- pyrimidin-2-ylamino)- phenyl]-benzamide |
| 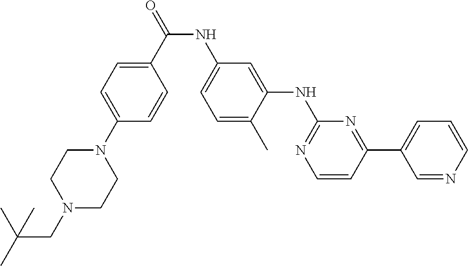 [SMILES: Cc1ccc(NC(=O)c2ccc(N3CCN(CC(C)(C)C)CC3)cc2)cc1Nc1nccc(-c2cccnc2)n1] | 4-[4-(2,2-Dimethyl- propyl)-piperazin-1- yl]-N-[4-methyl-3-(4- pyridin-3-yl-pyrimidin- 2-ylamino)-phenyl]- benzamide |
| 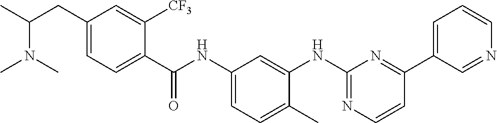 [SMILES: Cc1ccc(NC(=O)c2ccc(CC(C)N(C)C)cc2C(F)(F)F)cc1Nc1nccc(-c2cccnc2)n1] | 4-(2-Dimethylamino- propyl)-N-[4-methyl- 3-(4-pyridin-3-yl- pyrimidin-2-ylamino)- phenyl]-2-trifluoro- methyl-benzamide |
| 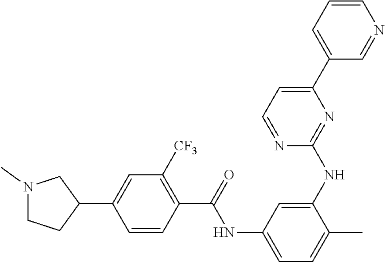 [SMILES: Cc1ccc(NC(=O)c2ccc(C3CCN(C)C3)cc2C(F)(F)F)cc1Nc1nccc(-c2cccnc2)n1] | N-[4-Methyl-3-(4- pyridin-3-yl-pyrimidin- 2-ylamino)-phenyl]- 4-(1-methyl-pyrrolidin- 3-yl)-2-trifluoromethyl- benzamide |
| 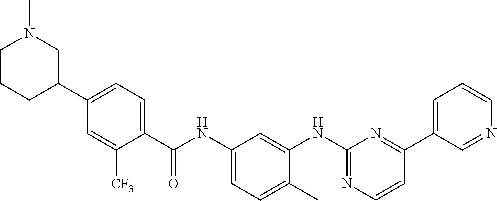 [SMILES: Cc1ccc(NC(=O)c2ccc(C3CCCN(C)C3)cc2C(F)(F)F)cc1Nc1nccc(-c2cccnc2)n1] | 4-(1-Methyl-piperidin- 3-yl)-N-[4-methyl-3-(4- pyridin-3-yl-pyrimidin- 2-ylamino)-phenyl]-2- trifluoromethyl-benz- amide |
| 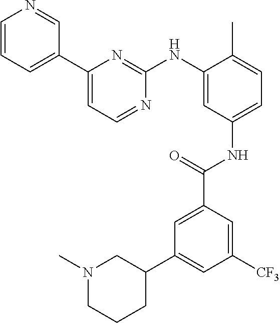 [SMILES: Cc1ccc(NC(=O)c2cc(C3CCCN(C)C3)cc(C(F)(F)F)c2)cc1Nc1nccc(-c2cccnc2)n1] | 3-(1-Methyl-piperidin- 3-yl)-N-[4-methyl-3- (4-pyridin-3-yl-pyrimi- din-2-ylamino)-phenyl]- 5-trifluoromethyl-benz- amide |
| 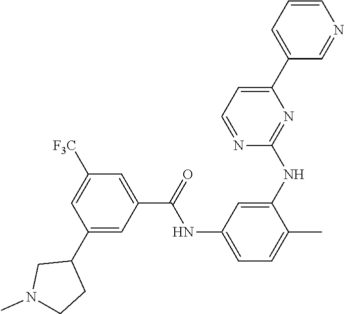 [SMILES: Cc1ccc(NC(=O)c2cc(C3CCN(C)C3)cc(C(F)(F)F)c2)cc1Nc1nccc(-c2cccnc2)n1] | N-[4-Methyl-3-(4- pyridin-3-yl-pyrimidin- 2-ylamino)-phenyl]- 3-(1-methyl-pyrrolidin- 3-yl)-5-trifluoromethyl- benzamide |
| 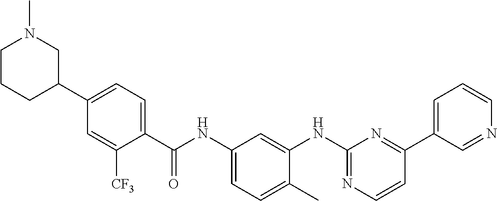 [SMILES: Cc1ccc(NC(=O)c2ccc(C3CCCN(C)C3)cc2C(F)(F)F)cc1Nc1nccc(-c2cccnc2)n1] | 4-(1-Ethyl-piperidin-3- yl)-N-[4-methyl-3-(4- pyridin-3-yl-pyrimidin- 2-ylamino)-phenyl]-2- trifluoromethyl-benz- amide |
| 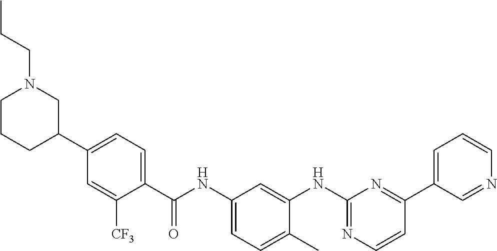 [SMILES: CCCN1CCCC(c2ccc(C(=O)Nc3ccc(C)c(Nc4nccc(-c5cccnc5)n4)c3)c(C(F)(F)F)c2)C1] | N-[4-Methyl-3-(4- pyridin-3-yl-pyrimidin- 2-ylamino)-phenyl]-4- (1-propyl-piperidin-3- yl)-2-trifluoromethyl- benzamide |
| 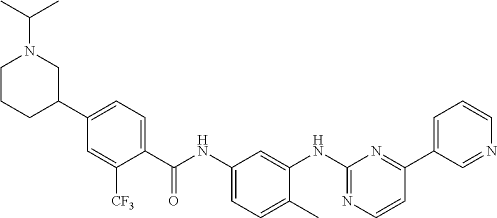 [SMILES: Cc1ccc(NC(=O)c2ccc(C3CCCN(C(C)C)C3)cc2C(F)(F)F)cc1Nc1nccc(-c2cccnc2)n1] | 4-(1-Isopropyl-piperi- din-3-yl)-N-[4-methyl- 3-(4-pyridin-3-yl- pyrimidin-2-ylamino)- phenyl]-2-trifluoro- methyl-benzamide |
| 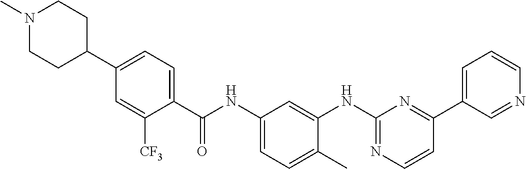 [SMILES: Cc1ccc(NC(=O)c2ccc(C3CCN(C)CC3)cc2C(F)(F)F)cc1Nc1nccc(-c2cccnc2)n1] | 4-(1-Methyl-piperidin- 4-yl)-N-[4-methyl-3- (4-pyridin-3-yl- pyrimidin-2-ylamino)- phenyl]-2-trifluoro- methyl-benzamide |
| 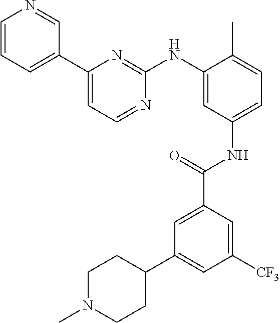 [SMILES: Cc1ccc(NC(=O)c2cc(C3CCN(C)CC3)cc(C(F)(F)F)c2)cc1Nc1nccc(-c2cccnc2)n1] | 3-(1-Methyl-piperidin- 4-yl)-N-[4-methyl-3-(4- pyridin-3-yl-pyrimidin- 2-ylamino)-phenyl]-5- trifluoromethyl-benz- amide |
| 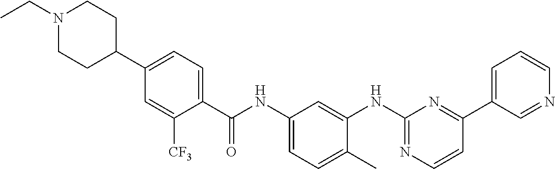 [SMILES: CCN1CCC(c2ccc(C(=O)Nc3ccc(C)c(Nc4nccc(-c5cccnc5)n4)c3)c(C(F)(F)F)c2)CC1] | 4-(1-Ethyl-piperidin-4- yl)-N-[4-methyl-3-(4- pyridin-3-yl-pyrimidin- 2-ylamino)-phenyl]-2- trifluoromethyl-benz- amide |
| 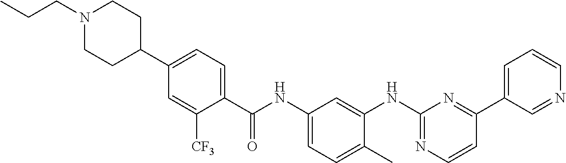 [SMILES: CCCN1CCC(c2ccc(C(=O)Nc3ccc(C)c(Nc4nccc(-c5cccnc5)n4)c3)c(C(F)(F)F)c2)CC1] | N-[4-Methyl-3-(4- pyridin-3-yl-pyrimidin- 2-ylamino)-phenyl]-4- (1-propyl-piperidin-4- yl)-2-trifluoromethyl- benzamide |
| 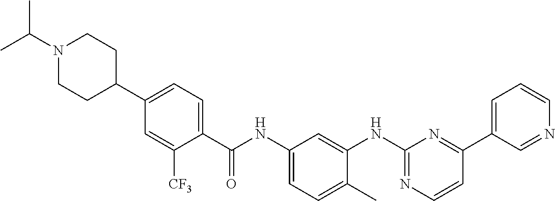 [SMILES: Cc1ccc(NC(=O)c2ccc(C3CCN(C(C)C)CC3)cc2C(F)(F)F)cc1Nc1nccc(-c2cccnc2)n1] | 4-(1-Isopropyl-piperi- din-4-yl)-N-[4-methyl- 3-(4-pyridin-3-yl- pyrimidin-2-ylamino)- phenyl]-2-trifluoro- methyl-benzamide |
| 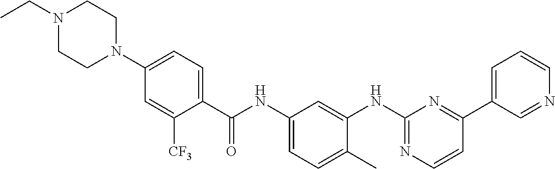 [SMILES: CCN1CCN(c2ccc(C(=O)Nc3ccc(C)c(Nc4nccc(-c5cccnc5)n4)c3)c(C(F)(F)F)c2)CC1] | 4-(4-Ethyl-piperazin-1- yl)-N-[4-methyl-3-(4- pyridin-3-yl-pyrimidin- 2-ylamino)-phenyl]-2- trifluoromethyl-benz- amide |
| 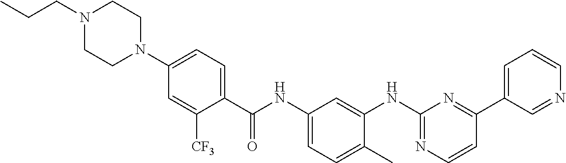 [SMILES: CCCN1CCN(c2ccc(C(=O)Nc3ccc(C)c(Nc4nccc(-c5cccnc5)n4)c3)c(C(F)(F)F)c2)CC1] | N-[4-Methyl-3-(4-pyridin-3-yl-pyrimidin- 2-ylamino)-phenyl]- 4-(4-propyl-piperazin- 1-yl)-2-trifluoromethyl- benzamide |
| 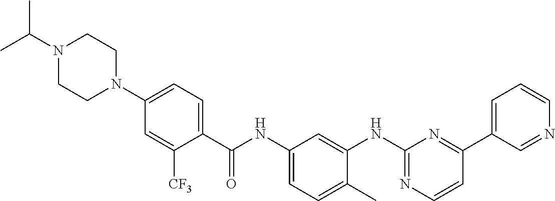 [SMILES: Cc1ccc(NC(=O)c2ccc(N3CCN(C(C)C)CC3)cc2C(F)(F)F)cc1Nc1nccc(-c2cccnc2)n1] | 4-(4-Isopropyl-pipera- zin-1-yl)-N-[4-methyl- 3-(4-pyridin-3-yl- pyrimidin-2-ylamino)- phenyl]-2-trifluoro- methyl-benzamide |
| 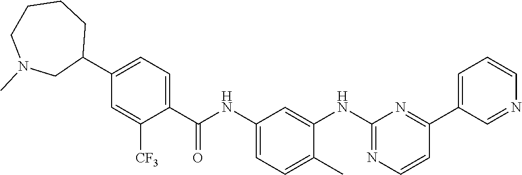 [SMILES: Cc1ccc(NC(=O)c2ccc(C3CCCCN(C)C3)cc2C(F)(F)F)cc1Nc1nccc(-c2cccnc2)n1] | 4-(1-Methyl-azepan-3- yl)-N-[4-methyl-3-(4- pyridin-3-yl-pyrimidin- 2-ylamino)-phenyl]-2- trifluoromethyl-benz- amide |
| 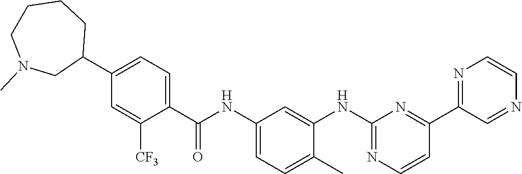 [SMILES: Cc1ccc(NC(=O)c2ccc(C3CCCCN(C)C3)cc2C(F)(F)F)cc1Nc1nccc(-c2cnccn2)n1] | 4-(1-Methyl-azepan-3- yl)-N-[4-methyl-3-(4- pyrazin-2-yl-pyrimidin- 2-ylamino)-phenyl]-2- trifluoromethyl-benz- amide |
| 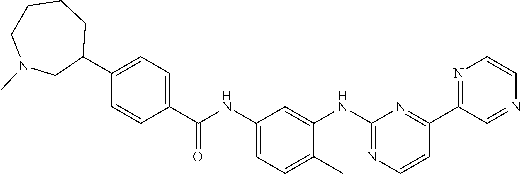 [SMILES: Cc1ccc(NC(=O)c2ccc(C3CCCCN(C)C3)cc2)cc1Nc1nccc(-c2cnccn2)n1] | 4-(1-Methyl-azepan-3- yl)-N-[4-methyl-3-(4- pyrazin-2-yl-pyrimidin- 2-ylamino)-phenyl]- benzamide |
| 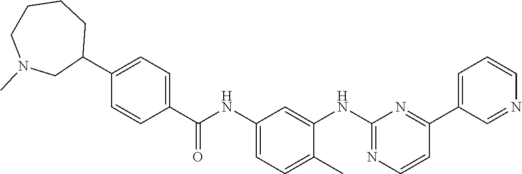 [SMILES: Cc1ccc(NC(=O)c2ccc(C3CCCCN(C)C3)cc2)cc1Nc1nccc(-c2cccnc2)n1] | 4-(1-Methyl-azepan-3- yl)-N-[4-methyl-3-(4- pyridin-3-yl-pyrimidin- 2-ylamino)-phenyl]- benzamide |
| 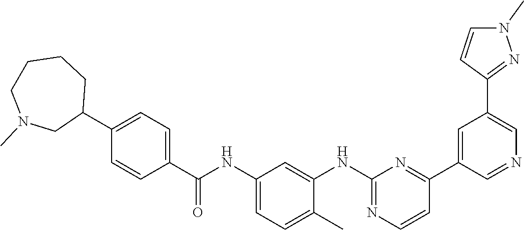 | 4-(1-Methyl-azepan-3- yl)-N-(4-methyl-3-{4- [5-(1-methyl-1H- pyrazol-3-yl)-pyridin- 3-yl]-pyrimidin-2-yl- amino}-phenyl)-benz- amide |
| 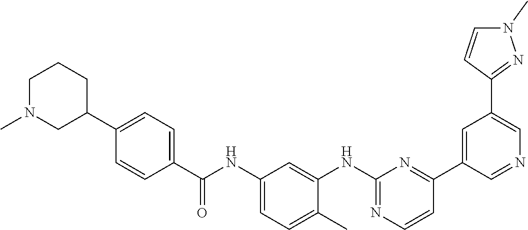 | N-(4-Methyl-3-{4-[5- (1-methyl-1H-pyrazol- 3-yl)-pyridin-3- pyrimidin-2-ylamino}- phenyl)-4-(1-methyl- piperidin-3-yl)-benz- amide |
| 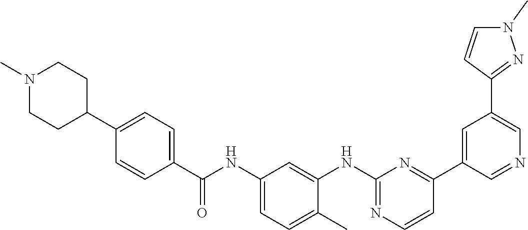 | N-(4-Methyl-3-{4-[5- (1-methyl-1H-pyrazol- 3-yl)-pyridin-3-yl]- pyrimidin-2-ylamino}- phenyl)-4-(1-methyl- piperidin-4-yl)-benz- amide |
| 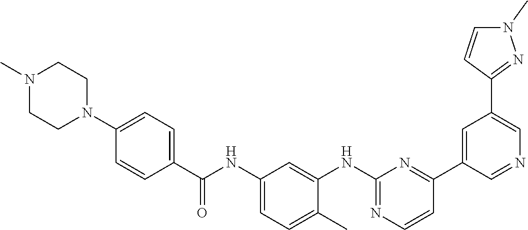 | N-(4-Methyl-3-{4-[5- (1-methyl-1H-pyrazol- 3-yl)-pyridin-3-yl]- pyrimidin-2-ylamino}- phenyl)-4-(4-methyl- piperazin-1-yl)-benz- amide |
| 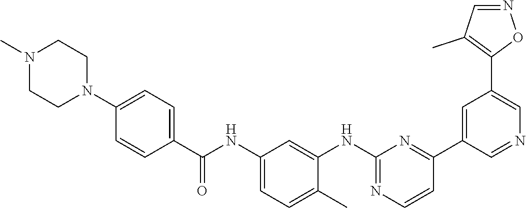 [SMILES: Cc1ccc(NC(=O)c2ccc(N3CCN(C)CC3)cc2)cc1Nc1nccc(-c2cncc(-c3oncc3C)c2)n1] | N-(4-Methyl-3-{4-[5- (4-methyl-isoxazol-5- yl)-pyridin-3-yl]- pyrimidin-2-ylamino}- phenyl)-4-(4-methyl- piperazin-1-yl)-benz- amide |
| 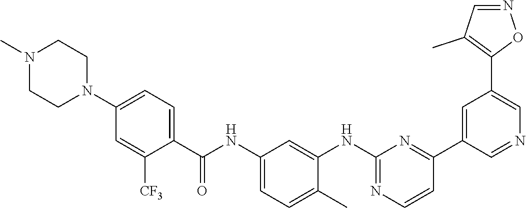 [SMILES: Cc1ccc(NC(=O)c2ccc(N3CCN(C)CC3)cc2C(F)(F)F)cc1Nc1nccc(-c2cncc(-c3oncc3C)c2)n1] | N-(4-Methyl-3-{4-[5- (4-methyl-isoxazol-5- yl)-pyridin-3-yl]- pyrimidin-2-ylamino}- phenyl)-4-(4-methyl- piperazin-1-yl)-2- trifluoromethyl-benz- amide |
| 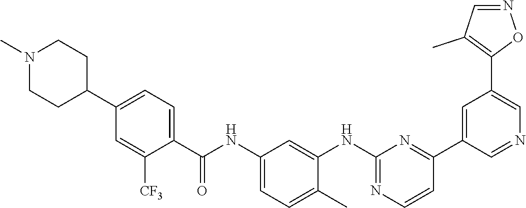 [SMILES: Cc1ccc(NC(=O)c2ccc(C3CCN(C)CC3)cc2C(F)(F)F)cc1Nc1nccc(-c2cncc(-c3oncc3C)c2)n1] | N-(4-Methyl-3-{4-[5- (4-methyl-isoxazol- 5-yl)-pyridin-3-yl]- pyrimidin-2-ylamino}- phenyl)-4-(1-methyl- piperidin-4-yl)-2- trifluoromethyl-benz- amide |
| 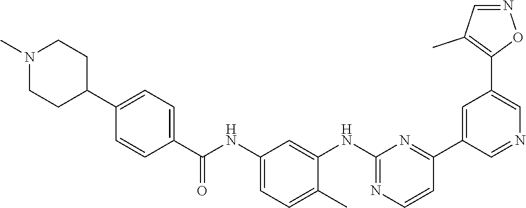 [SMILES: Cc1ccc(NC(=O)c2ccc(C3CCN(C)CC3)cc2)cc1Nc1nccc(-c2cncc(-c3oncc3C)c2)n1] | N-(4-Methyl-3-{4-[5- (4-methyl-isoxazol-5- yl)-pyridin-3-yl]- pyrimidin-2-ylamino}- phenyl)-4-(1-methyl- piperidin-4-yl)-benz- amide |
| 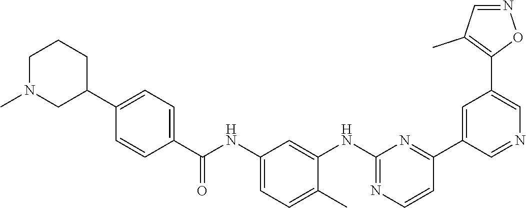 [SMILES: Cc1ccc(NC(=O)c2ccc(C3CCCN(C)C3)cc2)cc1Nc1nccc(-c2cncc(-c3oncc3C)c2)n1] | N-(4-Methyl-3-{4-[5- (4-methyl-isoxazol- 5-yl)-pyridin-3-yl]- pyrimidin-2-ylamino}- phenyl)-4-(1-methyl- piperidin-3-yl)-benz- amide |
| 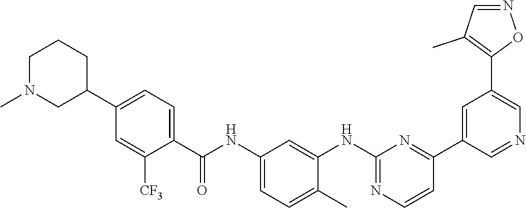 [SMILES: Cc1ccc(NC(=O)c2ccc(C3CCCN(C)C3)cc2C(F)(F)F)cc1Nc1nccc(-c2cncc(-c3oncc3C)c2)n1] | N-(4-Methyl-3-{4-[5- (4-methyl-isoxazol- 5-yl)-pyridin-3-yl]- pyrimidin-2-ylamino}- phenyl)-4-(1-methyl- piperidin-3-yl)-2- trifluoromethyl-benz- amide |
| 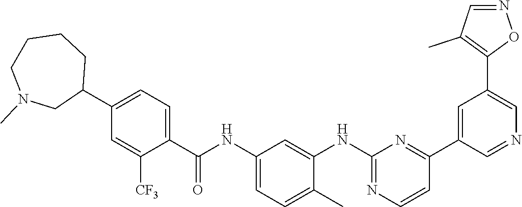 [SMILES: Cc1ccc(NC(=O)c2ccc(C3CCCCN(C)C3)cc2C(F)(F)F)cc1Nc1nccc(-c2cncc(-c3oncc3C)c2)n1] | 4-(1-Methyl-azepan-3- yl)-N-(4-methyl-3-{4- [5-(4-methyl-isoxazol- 5-yl)-pyridin-3-yl]- pyrimidin-2-ylamino}- phenyl)-2-trifluoro- methyl-benzamide |
| 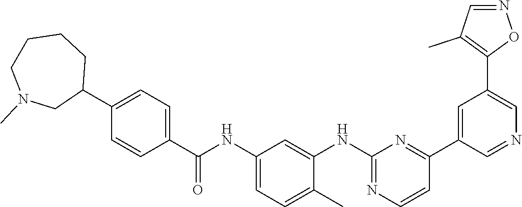 [SMILES: Cc1ccc(NC(=O)c2ccc(C3CCCCN(C)C3)cc2)cc1Nc1nccc(-c2cncc(-c3oncc3C)c2)n1] | 4-(1-Methyl-azepan-3- yl)-N-(4-methyl-3-{4- [5-(4-methyl-isoxazol- 5-yl)-pyridin-3-yl]- pyrimidin-2-ylamino}- phenyl)-benzamide |
| 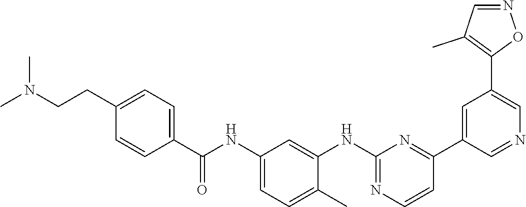 [SMILES: Cc1ccc(NC(=O)c2ccc(CCN(C)C)cc2)cc1Nc1nccc(-c2cncc(-c3oncc3C)c2)n1] | 4-(2-Dimethylamino- ethyl)-N-(4-methyl-3- 4-[5-(4-methyl-isoxazol- 5-yl)-pyridin-3-yl]- pyrimidin-2-ylamino}- phenyl)-benzamide |
| 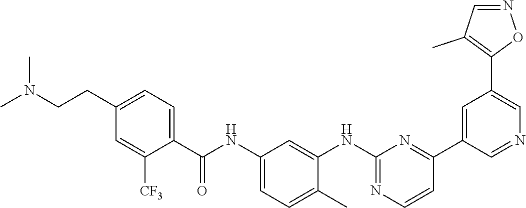 [SMILES: Cc1ccc(NC(=O)c2ccc(CCN(C)C)cc2C(F)(F)F)cc1Nc1nccc(-c2cncc(-c3oncc3C)c2)n1] | 4-(2-Dimethylamino-ethyl)-N-(4-methyl- 3-{4-[5-(4-methyl- isoxazol-5-yl)-pyridin- 3-yl]-pyrimidin-2- ylamino}-phenyl)-2- trifluoromethyl-benz- amide |
| 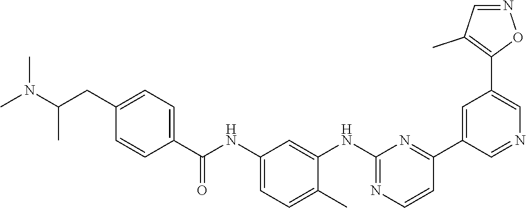 [SMILES: Cc1ccc(NC(=O)c2ccc(CC(C)N(C)C)cc2)cc1Nc1nccc(-c2cncc(-c3oncc3C)c2)n1] | 4-(2-Dimethylamino- propyl)-N-(4-methyl- 3-{4-[5-(4-methyl- isoxazol-5-yl)-pyridin- 3-yl]-pyrimidin-2- ylamino}-phenyl)- benzamide |
| 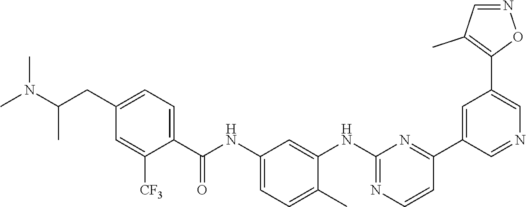 [SMILES: Cc1ccc(NC(=O)c2ccc(CC(C)N(C)C)cc2C(F)(F)F)cc1Nc1nccc(-c2cncc(-c3oncc3C)c2)n1] | 4-(2-Dimethylamino- propyl)-N-(4-methyl- 3-{4-[5-(4-methyl- isoxazol-5-yl)-pyridin- 3-yl]-pyrimidin-2- ylamino}-phenyl)-2- trifluoromethyl-benz- amide |
| 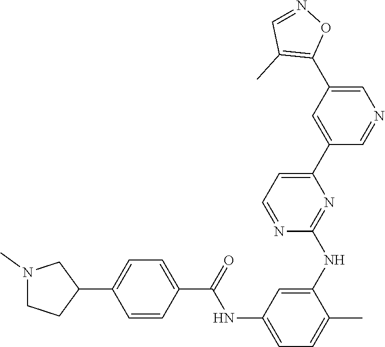 [SMILES: Cc1ccc(NC(=O)c2ccc(C3CCN(C)C3)cc2)cc1Nc1nccc(-c2cncc(-c3oncc3C)c2)n1] | N-(4-Methyl-3-{4-[5- (4-methyl-isoxazol-5- yl)-pyridin-3-yl]- pyrimidin-2-ylamino}- phenyl)-4-(1-methyl- pyrrolidin-3-yl)-benz- amide |
| 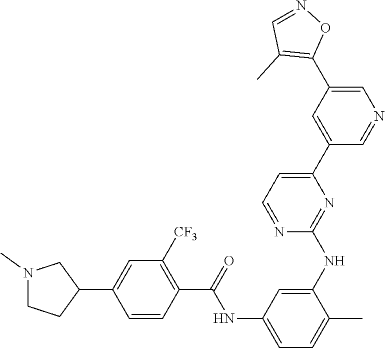 [SMILES: Cc1ccc(NC(=O)c2ccc(C3CCN(C)C3)cc2C(F)(F)F)cc1Nc1nccc(-c2cncc(-c3oncc3C)c2)n1] | N-(4-Methyl-3-{4-[5- (4-methyl-isoxazol-5- yl)-pyridin-3-yl]- pyrimidin-2-ylamino}- phenyl)-4-(1-methyl- pyrrolidin-3-yl)-2- trifluoromethyl-benz- amide |
| 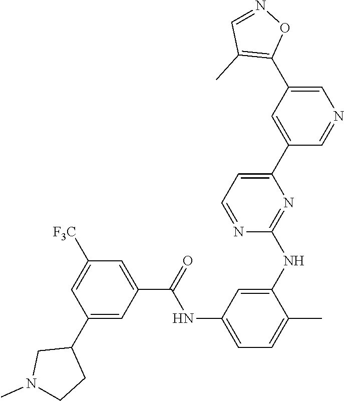 [SMILES: Cc1ccc(NC(=O)c2cc(C3CCN(C)C3)cc(C(F)(F)F)c2)cc1Nc1nccc(-c2cncc(-c3oncc3C)c2)n1] | N-(4-Methyl-3-{4-[5- (4-methyl-isoxazol-5- yl)-pyridin-3-yl]- pyrimidin-2-ylamino}- phenyl)-3-(1-methyl- pyrrolidin-3-yl)-5- trifluoromethyl-benz- amide |
| 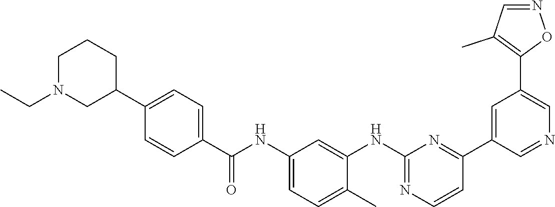 [SMILES: CCN1CCCC(c2ccc(C(=O)Nc3ccc(C)c(Nc4nccc(-c5cncc(-c6oncc6C)c5)n4)c3)cc2)C1] | 4-(1-Ethyl-piperidin-3- yl)-N-(4-methyl-3-{4- [5-(4-methyl-isoxazol- 5-yl)-pyridin-3-yl]- pyrimidin-2-ylamino}- phenyl)-benzamide |
| 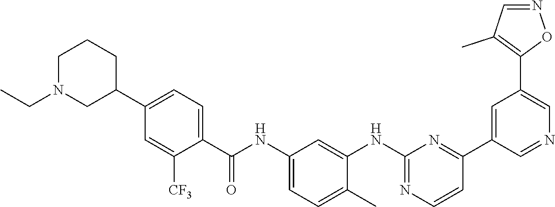 [SMILES: CCN1CCCC(c2ccc(C(=O)Nc3ccc(C)c(Nc4nccc(-c5cncc(-c6oncc6C)c5)n4)c3)c(C(F)(F)F)c2)C1] | 4-(1-Ethyl-piperidin-3- yl)-N-(4-methyl-3-{4- [5-(4-methyl-isoxazol- 5-yl)-pyridin-3-yl]- pyrimidin-2-ylamino}- phenyl)-2-trifluoro- methyl-benzamide |
| 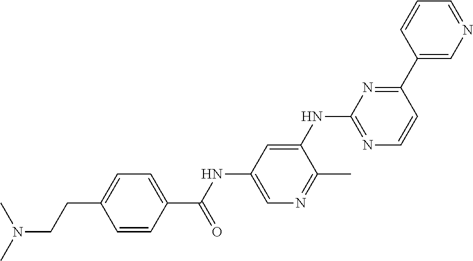 [SMILES: Cc1ncc(NC(=O)c2ccc(CCN(C)C)cc2)cc1Nc1nccc(-c2cccnc2)n1] | 4-(2-Dimethylamino- ethyl)-N-[6-methyl- 5-(4-pyridin-3-yl- pyrimidin-2-ylamino)- pyridin-3-yl]-benzamide |
| 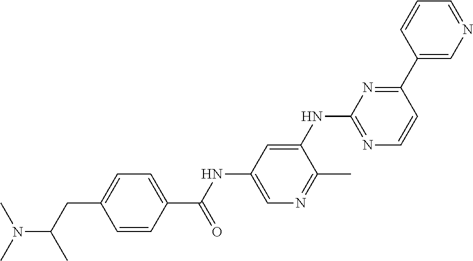 [SMILES: Cc1ncc(NC(=O)c2ccc(CC(C)N(C)C)cc2)cc1Nc1nccc(-c2cccnc2)n1] | 4-(2-Dimethylamino- propyl)-N-[6-methyl- 5-(4-pyridin-3-yl- pyrimidin-2-ylamino)- pyridin-3-yl]-benzamide |
| 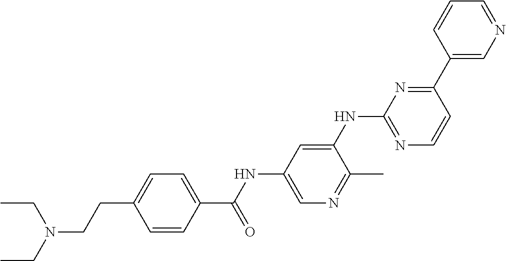 [SMILES: CCN(CC)CCc1ccc(C(=O)Nc2cnc(C)c(Nc3nccc(-c4cccnc4)n3)c2)cc1] | 4-(2-Diethylamino- ethyl)-N-[6-methyl-5- (4-pyridin-3-yl- pyrimidin-2-ylamino)- pyridin-3-yl]-benzamide |
| 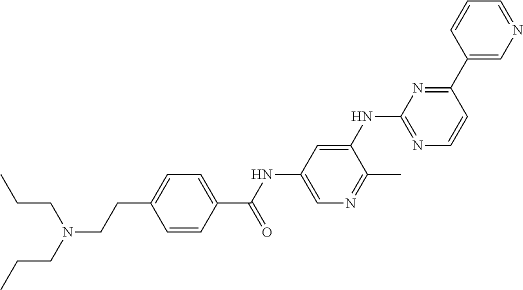 [SMILES: CCCN(CCC)CCc1ccc(C(=O)Nc2cnc(C)c(Nc3nccc(-c4cccnc4)n3)c2)cc1] | 4-(2-Dipropylamino-ethyl)-N-[6-methyl-5- (4-pyridin-3-yl- pyrimidin-2-ylamino)- pyridin-3-yl]-benzamide |
| 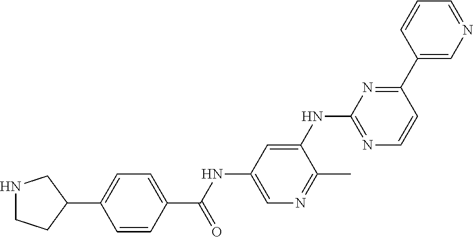 [SMILES: Cc1ncc(NC(=O)c2ccc(C3CCNC3)cc2)cc1Nc1nccc(-c2cccnc2)n1] | N-[6-Methyl-5-(4- pyridin-3-yl-pyrimidin- 2-ylamino)-pyridin-3- yl]-4-pyrrolidin-3-yl- benzamide |
| 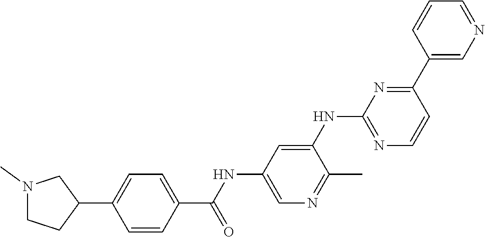 [SMILES: Cc1ncc(NC(=O)c2ccc(C3CCN(C)C3)cc2)cc1Nc1nccc(-c2cccnc2)n1] | N-[6-Methyl-5-(4- pyridin-3-yl-pyrimidin- 2-ylamino)-pyridin- 3-yl]-4-(1-methyl- pyrrolidin-3-yl)-benz- amide |
| 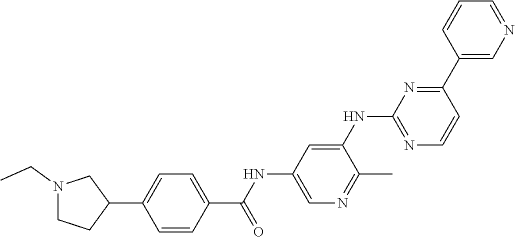 [SMILES: CCN1CCC(c2ccc(C(=O)Nc3cnc(C)c(Nc4nccc(-c5cccnc5)n4)c3)cc2)C1] | 4-(1-Ethyl-pyrrolidin- 3-yl)-N-[6-methyl-5- (4-pyridin-3-yl- pyrimidin-2-ylamino)- pyridin-3-yl]-benzamide |
| 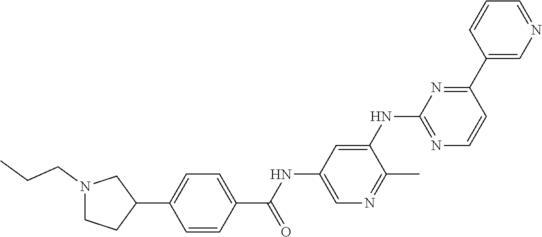 [SMILES: CCCN1CCC(c2ccc(C(=O)Nc3cnc(C)c(Nc4nccc(-c5cccnc5)n4)c3)cc2)C1] | N-[6-Methyl-5-(4- pyridin-3-yl-pyrimidin- 2-ylamino)-pyridin- 3-yl]-4-(1-propyl- pyrrolidin-3-yl)-benz- amide |
| 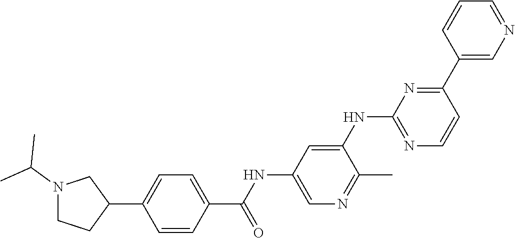 [SMILES: Cc1ncc(NC(=O)c2ccc(C3CCN(C(C)C)C3)cc2)cc1Nc1nccc(-c2cccnc2)n1] | 4-(1-Isopropyl-pyrroli- din-3-yl)-N-[6-methyl- 5-(4-pyridin-3-yl- pyrimidin-2-ylamino)- pyridin-3-yl]-benzamide |
| 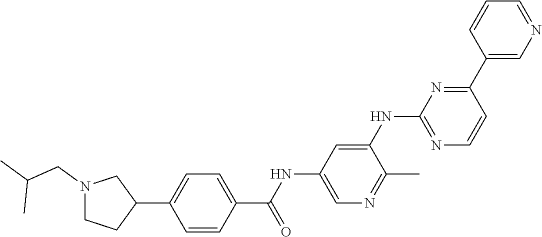 [SMILES: Cc1ncc(NC(=O)c2ccc(C3CCN(CC(C)C)C3)cc2)cc1Nc1nccc(-c2cccnc2)n1] | 4-(1-Isobutyl-pyrrolidin- 3-yl)-N-[6-methyl-5-(4- pyridin-3-yl-pyrimidin- 2-ylamino)-pyridin-3- yl]-benzamide |
| 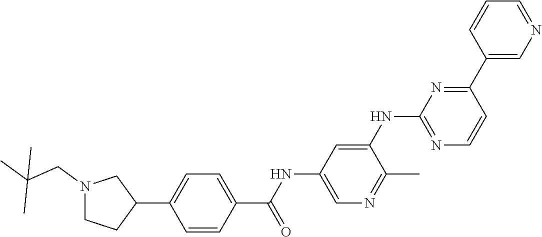 [SMILES: Cc1ncc(NC(=O)c2ccc(C3CCN(CC(C)(C)C)C3)cc2)cc1Nc1nccc(-c2cccnc2)n1] | 4-[1-(2,2-Dimethyl- propyl)-pyrrolidin-3- yl]-N-[6-methyl-5-(4- pyridin-3-yl-pyrimidin- 2-ylamino)-pyridin-3- yl]-benzamide |
| 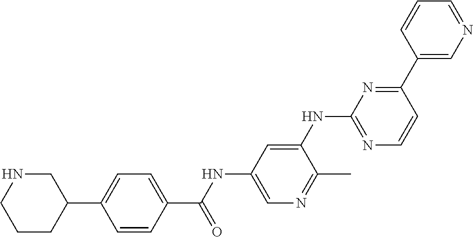 [SMILES: Cc1ncc(NC(=O)c2ccc(C3CCCNC3)cc2)cc1Nc1nccc(-c2cccnc2)n1] | N-[6-Methyl-5-(4- pyridin-3-yl-pyrimidin- 2-ylamino)-pyridin-3- yl]-4-piperidin-3-yl- benzamide |
| 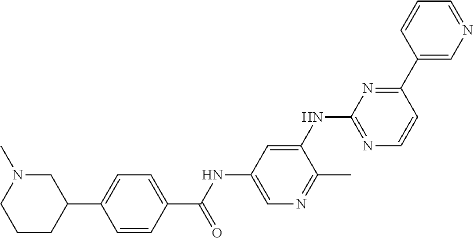 [SMILES: Cc1ncc(NC(=O)c2ccc(C3CCCN(C)C3)cc2)cc1Nc1nccc(-c2cccnc2)n1] | 4-(1-Methyl-piperidin- 3-yl)-N-[6-methyl-5-(4- pyridin-3-yl-pyrimidin- 2-ylamino)-pyridin-3- yl]-benzamide |
| 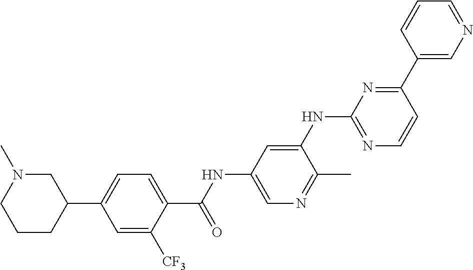 [SMILES: Cc1ncc(NC(=O)c2ccc(C3CCCN(C)C3)cc2C(F)(F)F)cc1Nc1nccc(-c2cccnc2)n1] | 4-(1-Methyl-piperidin- 3-yl)-N-[6-methyl-5-(4- pyridin-3-yl-pyrimidin- 2-ylamino)-pyridin-3- yl]-2-trifluoromethyl- benzamide |
| 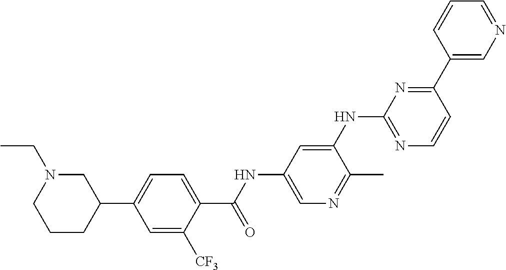 [SMILES: CCN1CCCC(c2ccc(C(=O)Nc3cnc(C)c(Nc4nccc(-c5cccnc5)n4)c3)c(C(F)(F)F)c2)C1] | 4-(1-Ethyl-piperidin-3- yl)-N-[6-methyl-5-(4- pyridin-3-yl-pyrimidin- 2-ylamino)-pyridin-3- yl]-2-trifluoromethyl- benzamide |
| 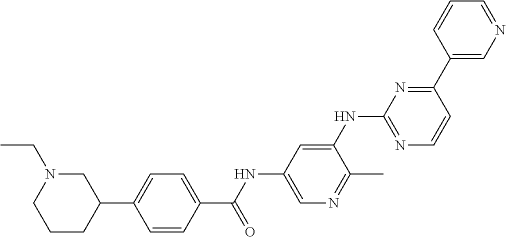 [SMILES: CCN1CCCC(c2ccc(C(=O)Nc3cnc(C)c(Nc4nccc(-c5cccnc5)n4)c3)cc2)C1] | 4-(1-Ethyl-piperidin-3- yl)-N-[6-methyl-5-(4- pyridin-3-yl-pyrimidin- 2-ylamino)-pyridin-3- yl]-benzamide |
| 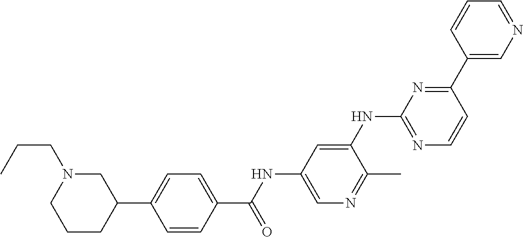 [SMILES: CCCN1CCCC(c2ccc(C(=O)Nc3cnc(C)c(Nc4nccc(-c5cccnc5)n4)c3)cc2)C1] | N-[6-Methyl-5-(4- pyridin-3-yl-pyrimidin- 2-ylamino)-pyridin-3- yl]-4-(1-propyl-piperi- din-3-yl)-benzamide |
| 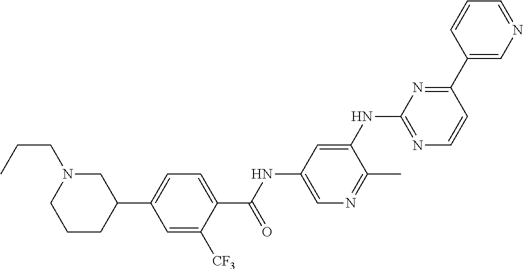 [SMILES: CCCN1CCCC(c2ccc(C(=O)Nc3cnc(C)c(Nc4nccc(-c5cccnc5)n4)c3)c(C(F)(F)F)c2)C1] | N-[6-Methyl-5-(4- pyridin-3-yl-pyrimidin- 2-ylamino)-pyridin-3- yl]-4-(1-propyl-piperi- din-3-yl)-2-trifluoro- methyl-benzamide |
| 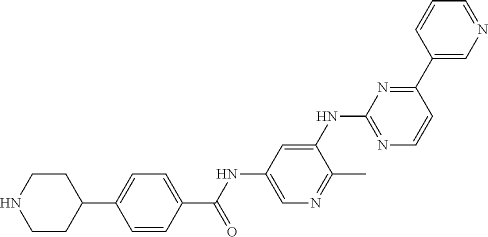 [SMILES: Cc1ncc(NC(=O)c2ccc(C3CCNCC3)cc2)cc1Nc1nccc(-c2cccnc2)n1] | N-[6-Methyl-5-(4- pyridin-3-yl-pyrimidin- 2-ylamino)-pyridin-3- yl]-4-piperidin-4-yl- benzamide |
| 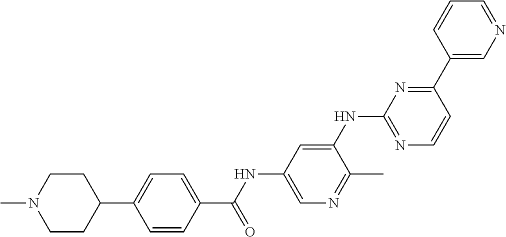 [SMILES: Cc1ncc(NC(=O)c2ccc(C3CCN(C)CC3)cc2)cc1Nc1nccc(-c2cccnc2)n1] | 4-(1-Methyl-piperidin- 4-yl)-N-[6-methyl-5-(4- pyridin-3-yl-pyrimidin- 2-ylamino)-pyridin-3- yl]-benzamide |
| 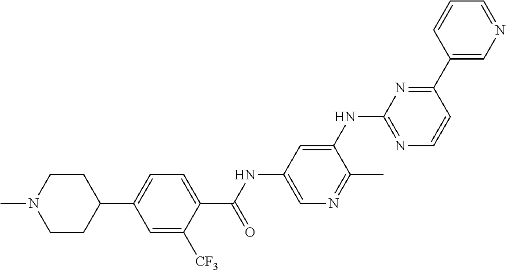 [SMILES: Cc1ncc(NC(=O)c2ccc(C3CCN(C)CC3)cc2C(F)(F)F)cc1Nc1nccc(-c2cccnc2)n1] | 4-(1-Methyl-piperidin- 4-yl)-N-[6-methyl-5- (4-pyridin-3-yl-pyrimi- din-2-ylamino)-pyridin- 3-yl]-2-trifluoromethyl- benzamide |
| 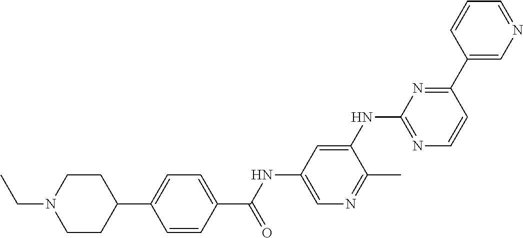 [SMILES: CCN1CCC(c2ccc(C(=O)Nc3cnc(C)c(Nc4nccc(-c5cccnc5)n4)c3)cc2)CC1] | 4-(1-Ethyl-piperidin-4- yl)-N-[6-methyl-5-(4- pyridin-3-yl-pyrimidin- 2-ylamino)-pyridin-3- yl]-benzamide |
| 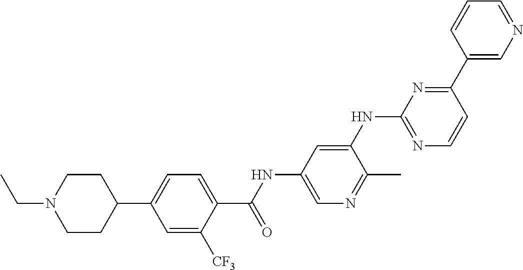 [SMILES: CCN1CCC(c2ccc(C(=O)Nc3cnc(C)c(Nc4nccc(-c5cccnc5)n4)c3)c(C(F)(F)F)c2)CC1] | 4-(1-Ethyl-piperidin-4- yl)-N-[6-methyl-5-(4- pyridin-3-yl-pyrimidin- 2-ylamino)-pyridin-3- yl]-2-trifluoromethyl- benzamide |
| 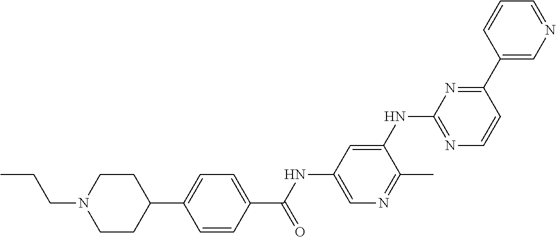 [SMILES: CCCN1CCC(c2ccc(C(=O)Nc3cnc(C)c(Nc4nccc(-c5cccnc5)n4)c3)cc2)CC1] | N-[6-Methyl-5-(4- pyridin-3-yl-pyrimidin- 2-ylamino)-pyridin-3- yl]-4-(1-propyl-piperi- din-4-yl)-benzamide |
| 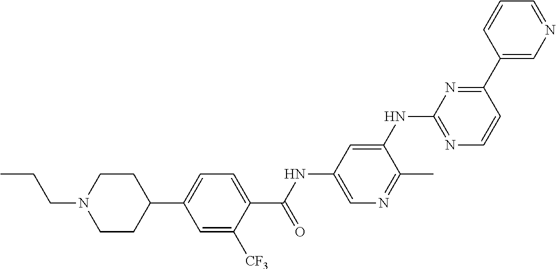 [SMILES: CCCN1CCC(c2ccc(C(=O)Nc3cnc(C)c(Nc4nccc(-c5cccnc5)n4)c3)c(C(F)(F)F)c2)CC1] | N-[6-Methyl-5-(4- pyridin-3-yl-pyrimidin- 2-ylamino)-pyridin-3- yl]-4-(1-propyl-piperi- din-4-yl)-2-trifluoro- methyl-benzamide |
| 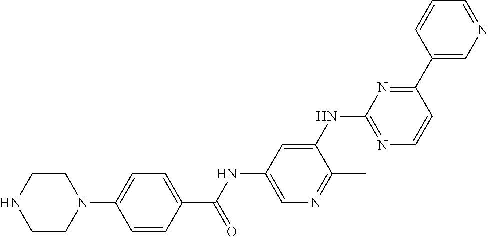 [SMILES: Cc1ncc(NC(=O)c2ccc(N3CCNCC3)cc2)cc1Nc1nccc(-c2cccnc2)n1] | N-[6-Methyl-5-(4- pyridin-3-yl-pyrimidin- 2-ylamino)-pyridin-3- yl]-4-piperazin-1-yl- benzamide |
| 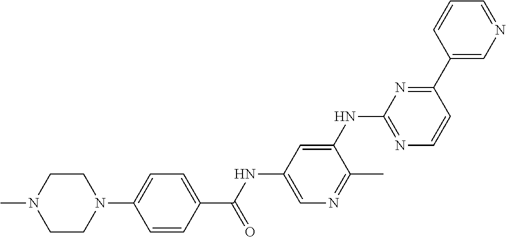 [SMILES: Cc1ncc(NC(=O)c2ccc(N3CCN(C)CC3)cc2)cc1Nc1nccc(-c2cccnc2)n1] | 4-(4-Methyl-piperazin- 1-yl)-N-[6-methyl-5-(4- pyridin-3-yl-pyrimidin- 2-ylamino)-pyridin-3- yl]-benzamide |
| 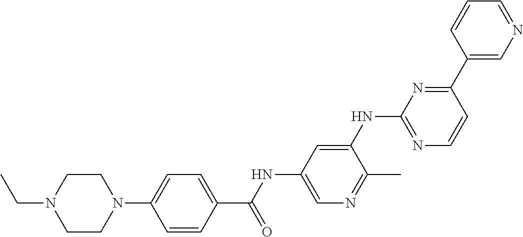 [SMILES: CCN1CCN(c2ccc(C(=O)Nc3cnc(C)c(Nc4nccc(-c5cccnc5)n4)c3)cc2)CC1] | 4-(4-Ethyl-piperazin- 1-yl)-N-[6-methyl- 5-(4-pyridin-3-yl- pyrimidin-2-ylamino)- pyridin-3-yl]-benzamide |
| 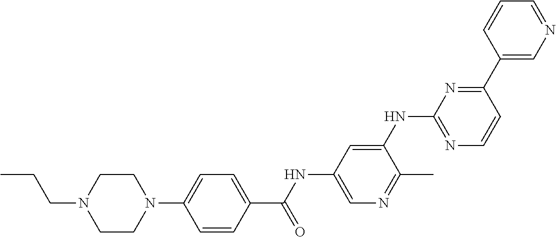 [SMILES: CCCN1CCN(c2ccc(C(=O)Nc3cnc(C)c(Nc4nccc(-c5cccnc5)n4)c3)cc2)CC1] | N-[6-Methyl-5-(4- pyridin-3-yl-pyrimidin- 2-ylamino)-pyridin-3- yl]-4-(4-propyl-pipera- zin-1-yl)-benzamide |
| 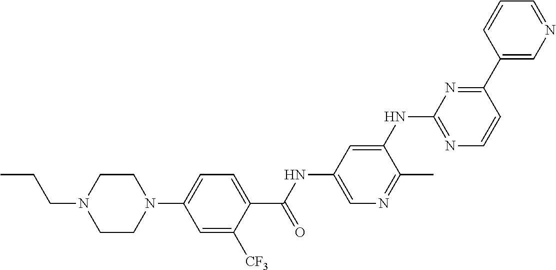 [SMILES: CCCN1CCN(c2ccc(C(=O)Nc3cnc(C)c(Nc4nccc(-c5cccnc5)n4)c3)c(C(F)(F)F)c2)CC1] | N-[6-Methyl-5-(4- pyridin-3-yl-pyrimidin- 2-ylamino)-pyridin-3- yl]-4-(4-propyl-pipera- zin-1-yl)-2-trifluoro- methyl-benzamide |
| 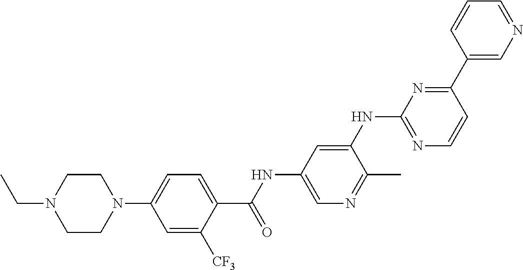 [SMILES: CCN1CCN(c2ccc(C(=O)Nc3cnc(C)c(Nc4nccc(-c5cccnc5)n4)c3)c(C(F)(F)F)c2)CC1] | 4-(4-Ethyl-piperazin- 1-yl)-N-[6-methyl- 5-(4-pyridin-3-yl- pyrimidin-2-ylamino)- pyridin-3-yl]-2- trifluoromethyl-benz- amide |
| 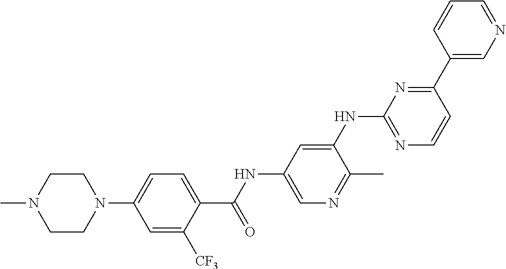 [SMILES: Cc1ncc(NC(=O)c2ccc(N3CCN(C)CC3)cc2C(F)(F)F)cc1Nc1nccc(-c2cccnc2)n1] | 4-(4-Methyl-piperazin- 1-yl)-N-[6-methyl-5- (4-pyridin-3-yl-pyrimi- din-2-ylamino)-pyridin- 3-yl]-2-trifluoromethyl- benzamide |
| 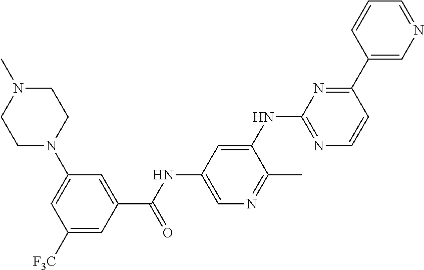 [SMILES: Cc1ncc(NC(=O)c2cc(N3CCN(C)CC3)cc(C(F)(F)F)c2)cc1Nc1nccc(-c2cccnc2)n1] | 3-(4-Methyl-piperazin- 1-yl)-N-[6-methyl-5- (4-pyridin-3-yl-pyrimi- din-2-ylamino)-pyridin- 3-yl]-5-trifluorornethyl- benzamide |
| 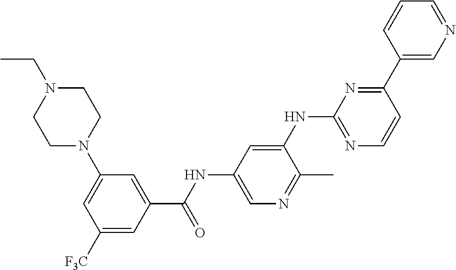 [SMILES: CCN1CCN(c2cc(C(=O)Nc3cnc(C)c(Nc4nccc(-c5cccnc5)n4)c3)cc(C(F)(F)F)c2)CC1] | 3-(4-Ethyl-piperazin- 1-yl)-N-[6-methyl-5- (4-pyridin-3-yl-pyrimi- din-2-ylamino)-pyridin- 3-yl]-5-trifluoromethyl- benzamide |
| 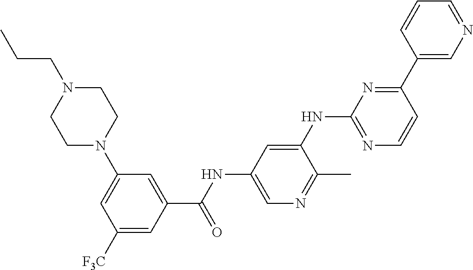 [SMILES: CCCN1CCN(c2cc(C(=O)Nc3cnc(C)c(Nc4nccc(-c5cccnc5)n4)c3)cc(C(F)(F)F)c2)CC1] | N-[6-Methyl-5-(4- pyridin-3-yl-pyrimidin- 2-ylamino)-pyridin- 3-yl]-3-(4-propyl- piperazin-1-yl)-5- trifluoromethyl-benz- amide |
| 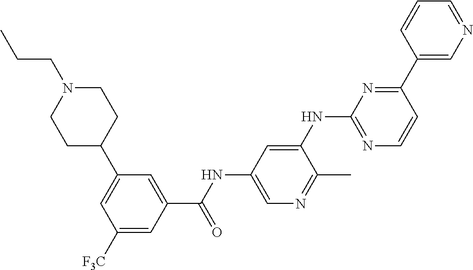 [SMILES: CCCN1CCC(c2cc(C(=O)Nc3cnc(C)c(Nc4nccc(-c5cccnc5)n4)c3)cc(C(F)(F)F)c2)CC1] | N-[6-Methyl-5-(4- pyridin-3-yl-pyrimidin- 2-ylamino)-pyridin-3- yl]-3-(1-propyl-piperi- din-4-yl)-5-trifluoro- methyl-benzamide |
| 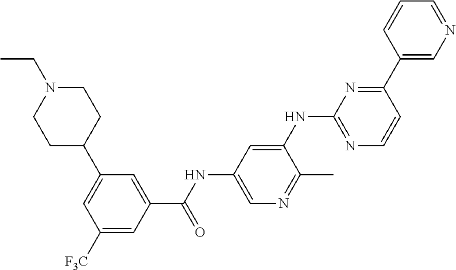 [SMILES: CCN1CCC(c2cc(C(=O)Nc3cnc(C)c(Nc4nccc(-c5cccnc5)n4)c3)cc(C(F)(F)F)c2)CC1] | 3-(1-Ethyl-piperidin-4- yl)-N-[6-methyl-5-(4- pyridin-3-yl-pyrimidin- 2-ylamino)-pyridin-3- yl]-5-trifluoromethyl- benzamide |
| 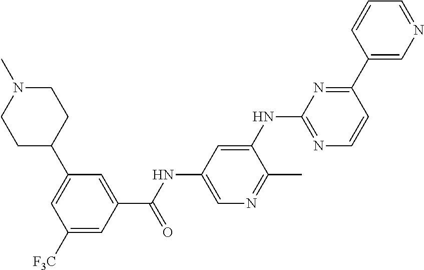 [SMILES: Cc1ncc(NC(=O)c2cc(C3CCN(C)CC3)cc(C(F)(F)F)c2)cc1Nc1nccc(-c2cccnc2)n1] | 3-(1-Methyl-piperidin- 4-yl)-N-[6-methyl-5-(4- pyridin-3-yl-pyrimidin- 2-ylamino)-pyridin-3- yl]-5-trifluoromethyl- benzamide |
| 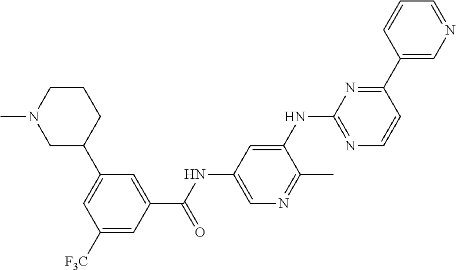 [SMILES: Cc1ncc(NC(=O)c2cc(C3CCCN(C)C3)cc(C(F)(F)F)c2)cc1Nc1nccc(-c2cccnc2)n1] | 3-(1-Methyl-piperidin- 3-yl)-N-[6-methyl-5-(4- pyridin-3-yl-pyrimidin- 2-ylamino)-pyridin-3- yl]-5-trifluoromethyl- benzamide |
| 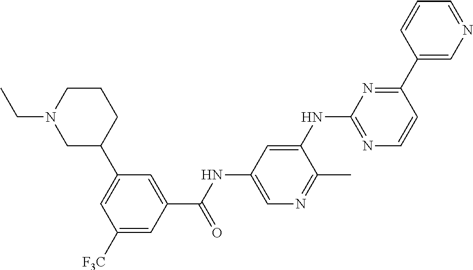 [SMILES: CCN1CCCC(c2cc(C(=O)Nc3cnc(C)c(Nc4nccc(-c5cccnc5)n4)c3)cc(C(F)(F)F)c2)C1] | 3-(1-Ethyl-piperidin-3- yl)-N-[6-methyl-5-(4- pyridin-3-yl-pyrimidin- 2-ylamino)-pyridin-3- yl]-5-trifluoromethyl- benzamide |
| 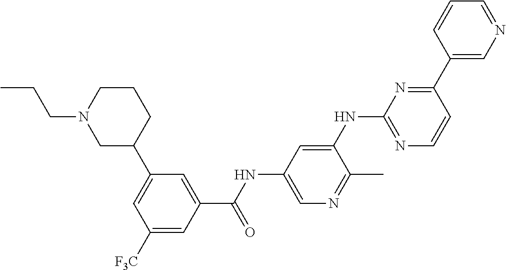 [SMILES: CCCN1CCCC(c2cc(C(=O)Nc3cnc(C)c(Nc4nccc(-c5cccnc5)n4)c3)cc(C(F)(F)F)c2)C1] | N-[6-Methyl-5-(4- pyridin-3-yl-pyrimidin- 2-ylamino)-pyridin-3- yl]-3-(1-propyl-piperi- din-3-yl)-5-trifluoro- methyl-benzamide |

In some embodiments, the compounds of the present invention are represented in Formula (III), or a pharmaceutically acceptable salt, solvate, hydrate, cocrystal, or prodrug thereof, wherein X is CH or nitrogen; Y is CH or N; R₄ is hydrogen or CF₃; and R₃ and R₅ are the same as defined in Formula (I). Examples of compounds of Formula (III) are listed in Table 2.

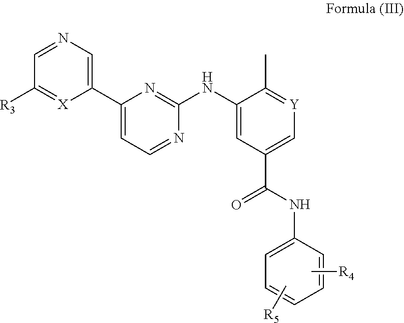

TABLE 2
Embodiments of Formula (III)

| Structure | Name |
|---|---|
| 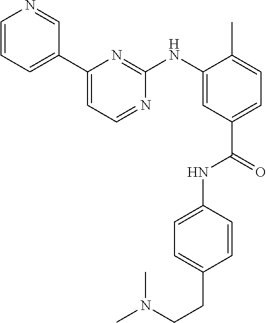 [SMILES: Cc1ccc(C(=O)Nc2ccc(CCN(C)C)cc2)cc1Nc1nccc(-c2cccnc2)n1] | N-[4-(2-Dimethylamino-ethyl)-phenyl]-4-methyl-3-(4-pyridin- 3-yl-pyrimidin-2-ylamino)-benzamide |
| 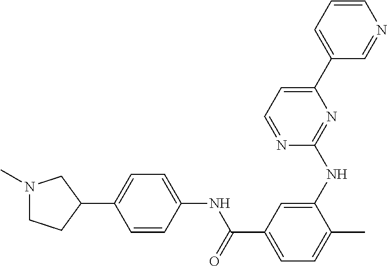 [SMILES: Cc1ccc(C(=O)Nc2ccc(C3CCN(C)C3)cc2)cc1Nc1nccc(-c2cccnc2)n1] | 4-Methyl-N-[4-(1-methyl-pyrrolidin-3-yl)-phenyl]-3-(4-pyridin- 3-yl-pyrimidin-2-ylamino)-benzamide |
| 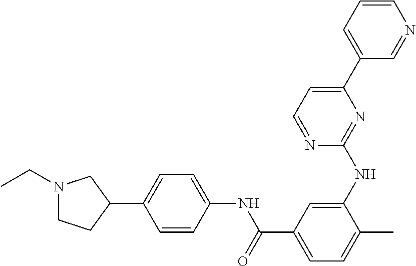 [SMILES: CCN1CCC(c2ccc(NC(=O)c3ccc(C)c(Nc4nccc(-c5cccnc5)n4)c3)cc2)C1] | N-[4-(1-Ethyl-pyrrolidin-3-yl)-phenyl]-4-methyl-3-(4-pyridin-3- yl-pyrimidin-2-ylamino)-benzamide |
| 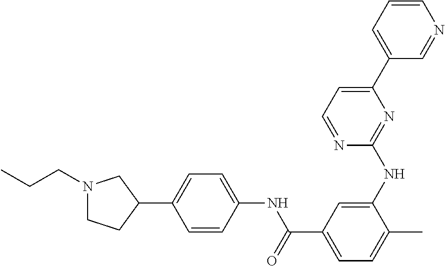 [SMILES: CCCN1CCC(c2ccc(NC(=O)c3ccc(C)c(Nc4nccc(-c5cccnc5)n4)c3)cc2)C1] | 4-Methyl-N-[4-(1-propyl-pyrrolidin-3-yl)-phenyl]-3-(4-pyridin- 3-yl-pyrimidin-2-ylamino)-benzamide |
| 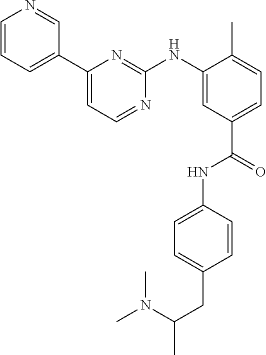 [SMILES: Cc1ccc(C(=O)Nc2ccc(CC(C)N(C)C)cc2)cc1Nc1nccc(-c2cccnc2)n1] | N-[4-(2-Dimethylamino-propyl)-phenyl]-4-methyl-3-(4-pyridin- 3-yl-pyrimidin-2-ylamino)-benzamide |
| 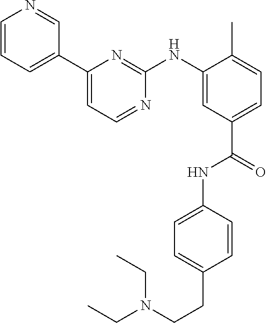 [SMILES: CCN(CC)CCc1ccc(NC(=O)c2ccc(C)c(Nc3nccc(-c4cccnc4)n3)c2)cc1] | N-[4-(2-Diethylamino-ethyl)-phenyl]-4-methyl-3-(4-pyridin-3- yl-pyrimidin-2-ylamino)-benzamide |
| 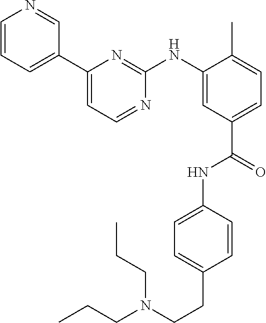 [SMILES: CCCN(CCC)CCc1ccc(NC(=O)c2ccc(C)c(Nc3nccc(-c4cccnc4)n3)c2)cc1] | N-[4-(2-Propylamino-ethyl)-phenyl]-4-methyl-3-(4-pyridin-3- yl-pyrimidin-2-ylamino)-benzamide |
| 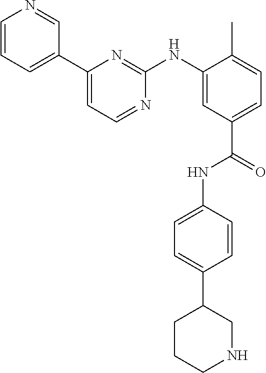 [SMILES: Cc1ccc(C(=O)Nc2ccc(C3CCCNC3)cc2)cc1Nc1nccc(-c2cccnc2)n1] | 4-Methyl-N-(4-piperidin-3-yl-phenyl)-3-(4-pyridin-3-yl- pyrimidin-2-ylamino)-benzamide |
| 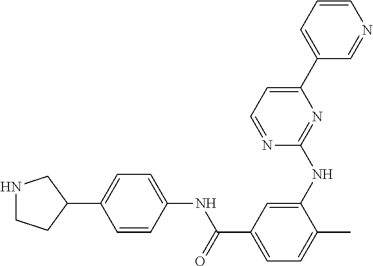 [SMILES: Cc1ccc(C(=O)Nc2ccc(C3CCNC3)cc2)cc1Nc1nccc(-c2cccnc2)n1] | 4-Methyl-3-(4-pyridin-3-yl-pyrimidin-2-ylamino)-N-(4- pyrrolidin-3-yl-phenyl)-benzamide |
| 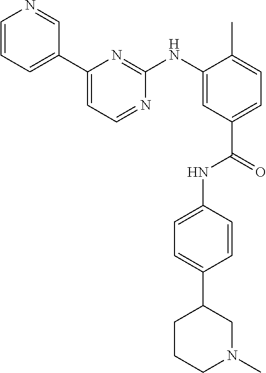 [SMILES: Cc1ccc(C(=O)Nc2ccc(C3CCCN(C)C3)cc2)cc1Nc1nccc(-c2cccnc2)n1] | 4-Methyl-N-[4-(1-methyl-piperidin-3-yl)-phenyl]-3-(4-pyridin- 3-yl-pyrimidin-2-ylamino)-benzamide |
| 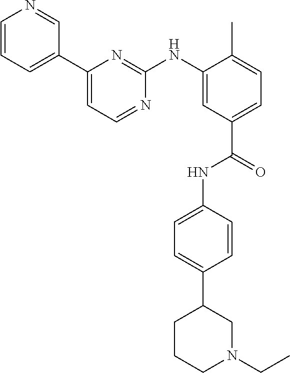 [SMILES: CCN1CCCC(c2ccc(NC(=O)c3ccc(C)c(Nc4nccc(-c5cccnc5)n4)c3)cc2)C1] | N-[4-(1-Ethyl-piperidin-3-yl)-phenyl]-4-methyl-3-(4-pyridin-3- yl-pyrimidin-2-ylamino)-benzamide |
| 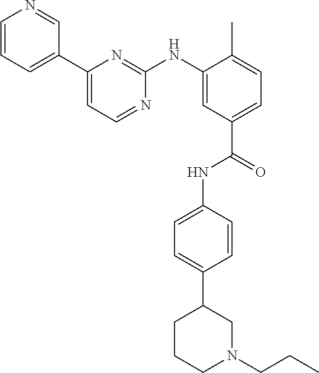 [SMILES: CCCN1CCCC(c2ccc(NC(=O)c3ccc(C)c(Nc4nccc(-c5cccnc5)n4)c3)cc2)C1] | 4-Methyl-N-[4-(1-propyl-piperidin-3-yl)-phenyl]-3-(4-pyridin- 3-yl-pyrimidin-2-ylamino)-benzamide |
| 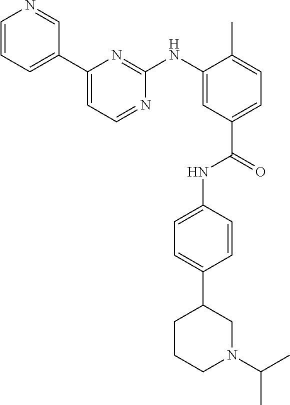 [SMILES: Cc1ccc(C(=O)Nc2ccc(C3CCCN(C(C)C)C3)cc2)cc1Nc1nccc(-c2cccnc2)n1] | N-[4-(1-Isopropyl-piperidin-3-yl)-phenyl]-4-methyl-3-(4- pyridin-3-yl-pyrimidin-2-ylamino)-benzamide |
| 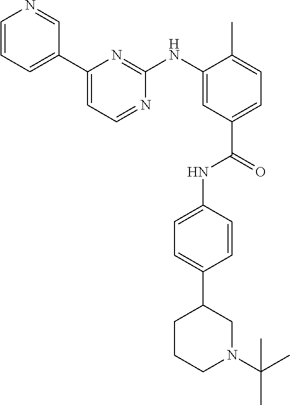 [SMILES: Cc1ccc(C(=O)Nc2ccc(C3CCCN(C(C)(C)C)C3)cc2)cc1Nc1nccc(-c2cccnc2)n1] | N-[4-(1-tert-Butyl-piperidin-3-yl)-phenyl]-4-methyl-3-(4- pyridin-3-yl-pyrimidin-2-ylamino)-benzamide |
| 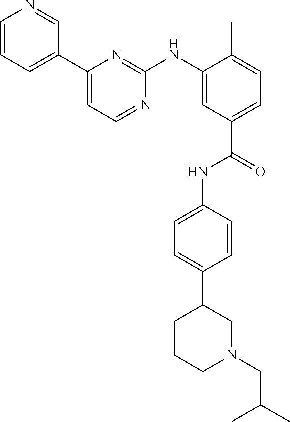 [SMILES: Cc1ccc(C(=O)Nc2ccc(C3CCCN(CC(C)C)C3)cc2)cc1Nc1nccc(-c2cccnc2)n1] | N-[4-(1-Isobutyl-piperidin-3-yl)-phenyl]-4-methyl-3-(4-pyridin- 3-yl-pyrimidin-2-ylamino)-benzamide |
| 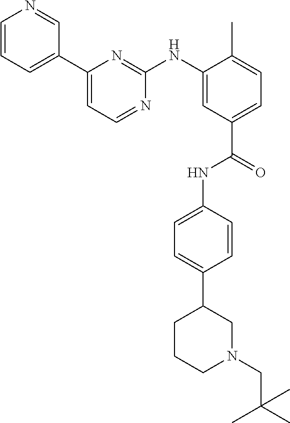 [SMILES: Cc1ccc(C(=O)Nc2ccc(C3CCCN(CC(C)(C)C)C3)cc2)cc1Nc1nccc(-c2cccnc2)n1] | N-{4-[1-(2,2-Dimethyl-propyl)-piperidin-3-yl]-phenyl}-4- methyl-3-(4-pyridin-3-yl-pyrimidin-2-ylamino)-benzamide |
| 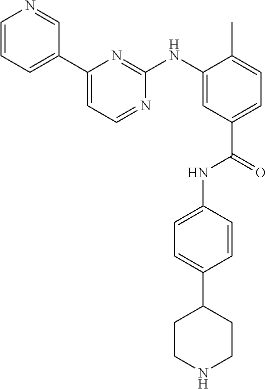 [SMILES: Cc1ccc(C(=O)Nc2ccc(C3CCNCC3)cc2)cc1Nc1nccc(-c2cccnc2)n1] | 4-Methyl-N-(4-piperidin-4-yl-phenyl)-3-(4-pyridin-3-yl- pyrimidin-2-ylamino)-benzamide |
| 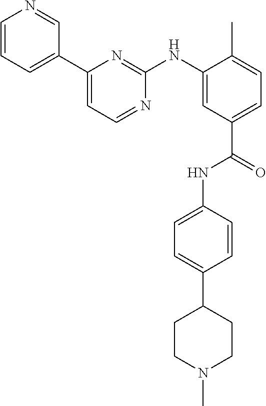 [SMILES: Cc1ccc(C(=O)Nc2ccc(C3CCN(C)CC3)cc2)cc1Nc1nccc(-c2cccnc2)n1] | 4-Methyl-N-[4-(1-methyl-piperidin-4-yl)-phenyl]-3-(4-pyridin- 3-yl-pyrimidin-2-ylamino)-benzamide |
| 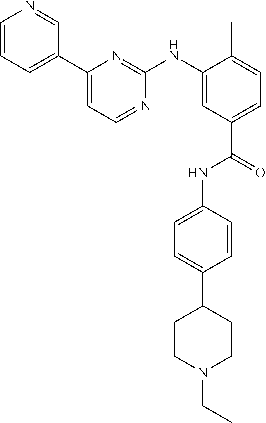 [SMILES: CCN1CCC(c2ccc(NC(=O)c3ccc(C)c(Nc4nccc(-c5cccnc5)n4)c3)cc2)CC1] | N-[4-(1-Ethyl-piperidin-4-yl)-phenyl]-4-methyl-3-(4-pyridin-3- yl-pyrimidin-2-ylamino)-benzamide |
| 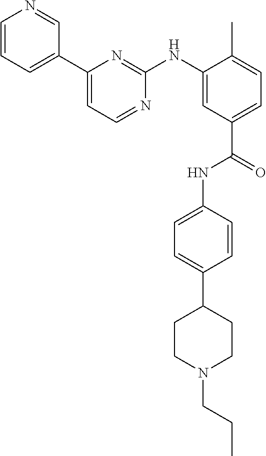 [SMILES: CCCN1CCC(c2ccc(NC(=O)c3ccc(C)c(Nc4nccc(-c5cccnc5)n4)c3)cc2)CC1] | 4-Methyl-N-[4-(1-propyl-piperidin-4-yl)-phenyl]-3-(4-pyridin- 3-yl-pyrimidin-2-ylamino)-benzamide |
| 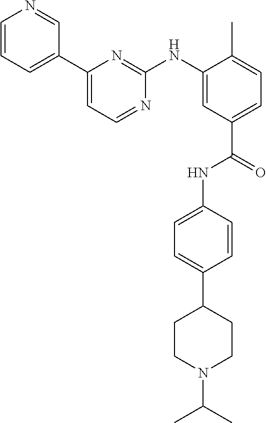 [SMILES: Cc1ccc(C(=O)Nc2ccc(C3CCN(C(C)C)CC3)cc2)cc1Nc1nccc(-c2cccnc2)n1] | N-[4-(1-Isopropyl-piperidin-4-yl)-phenyl]-4-methyl-3-(4- pyridin-3-yl-pyrimidin-2-ylamino)-benzamide |
| 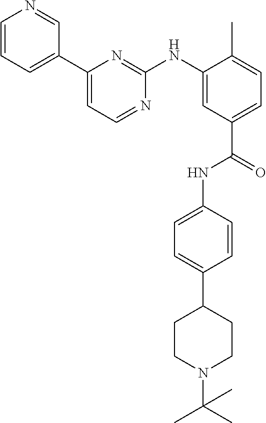 [SMILES: Cc1ccc(C(=O)Nc2ccc(C3CCN(C(C)(C)C)CC3)cc2)cc1Nc1nccc(-c2cccnc2)n1] | N-[4-(1-tert-Butyl-piperidin-4-yl)-phenyl]-4-methyl-3-(4- pyridin-3-yl-pyrimidin-2-ylamino)-benzamide |
| 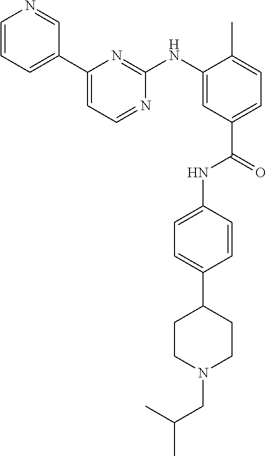 [SMILES: Cc1ccc(C(=O)Nc2ccc(C3CCN(CC(C)C)CC3)cc2)cc1Nc1nccc(-c2cccnc2)n1] | N-[4-(1-Isobutyl-piperidin-4-yl)-phenyl]-4-methyl-3-(4-pyridin- 3-yl-pyrimidin-2-ylamino)-benzamide |
| 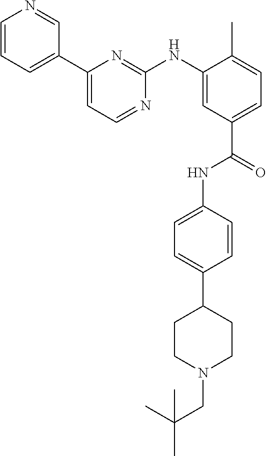 [SMILES: Cc1ccc(C(=O)Nc2ccc(C3CCN(CC(C)(C)C)CC3)cc2)cc1Nc1nccc(-c2cccnc2)n1] | N-{4-[1-(2,2-Dimethyl-propyl)-piperidin-4-yl]-phenyl}-4- methyl-3-(4-pyridin-3-yl-pyrimidin-2-ylamino)-benzamide |
| 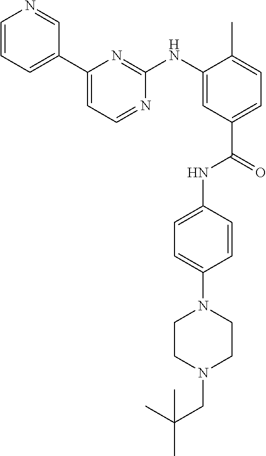 [SMILES: Cc1ccc(C(=O)Nc2ccc(N3CCN(CC(C)(C)C)CC3)cc2)cc1Nc1nccc(-c2cccnc2)n1] | N-{4-[4-(2,2-Dimethyl-propyl)-piperazin-1-yl]-phenyl}-4- methyl-3-(4-pyridin-3-yl-pyrimidin-2-ylamino)-benzamide |
| 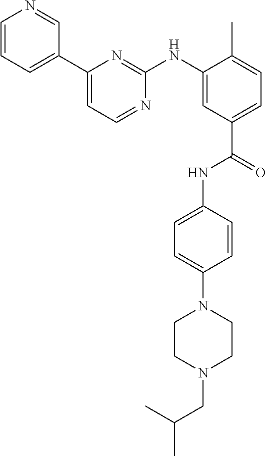 [SMILES: Cc1ccc(C(=O)Nc2ccc(N3CCN(CC(C)C)CC3)cc2)cc1Nc1nccc(-c2cccnc2)n1] | N-[4-(4-Isobutyl-piperazin-1-yl)-phenyl]-4-methyl-3-(4-pyridin- 3-yl-pyrimidin-2-ylamino)-benzamide |
| 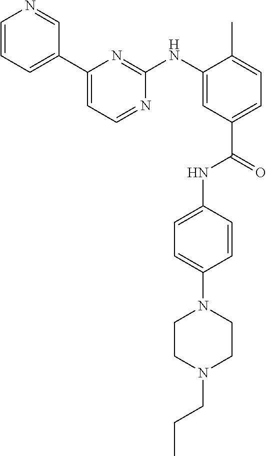 [SMILES: CCCN1CCN(c2ccc(NC(=O)c3ccc(C)c(Nc4nccc(-c5cccnc5)n4)c3)cc2)CC1] | 4-Methyl-N-[4-(4-propyl-piperazin-1-yl)-phenyl]-3-(4-pyridin- 3-yl-pyrimidin-2-ylamino)-benzamide |
| 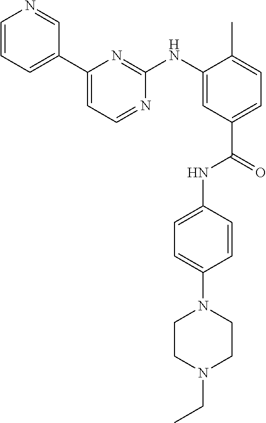 [SMILES: CCN1CCN(c2ccc(NC(=O)c3ccc(C)c(Nc4nccc(-c5cccnc5)n4)c3)cc2)CC1] | N-[4-(4-Ethyl-piperazin-1-yl)-phenyl]-4-methyl-3-(4-pyridin-3- yl-pyrimidin-2-ylamino)-benzamide |
| 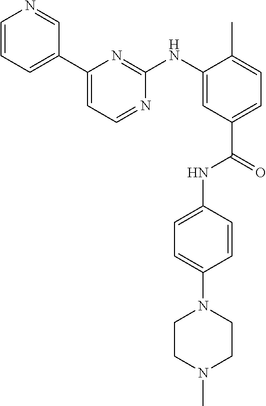 [SMILES: Cc1ccc(C(=O)Nc2ccc(N3CCN(C)CC3)cc2)cc1Nc1nccc(-c2cccnc2)n1] | 4-Methyl-N-[4-(4-methyl-piperazin-1-yl)-phenyl]-3-(4-pyridin- 3-yl-pyrimidin-2-ylamino)-benzamide |
| 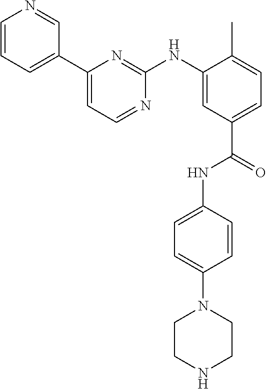 [SMILES: Cc1ccc(C(=O)Nc2ccc(N3CCNCC3)cc2)cc1Nc1nccc(-c2cccnc2)n1] | 4-Methyl-N-(4-piperazin-1-yl-phenyl)-3-(4-pyridin-3-yl- pyrimidin-2-ylamino)-benzamide |
| 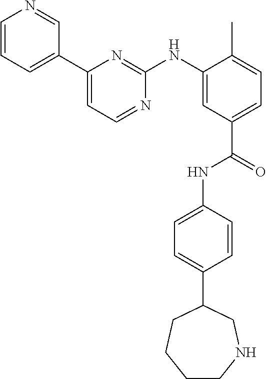 [SMILES: Cc1ccc(C(=O)Nc2ccc(C3CCCCNC3)cc2)cc1Nc1nccc(-c2cccnc2)n1] | N-(4-Azepan-3-yl-phenyl)-4-methyl-3-(4-pyridin-3-yl- pyrimidin-2-ylamino)-benzamide |
| 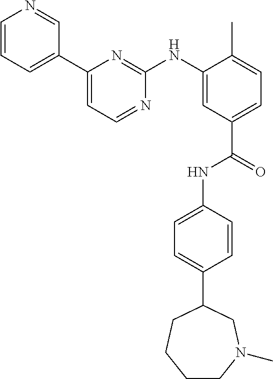 [SMILES: Cc1ccc(C(=O)Nc2ccc(C3CCCCN(C)C3)cc2)cc1Nc1nccc(-c2cccnc2)n1] | 4-Methyl-N-[4-(1-methyl-azepan-3-yl)-phenyl]-3-(4-pyridin-3- yl-pyrimidin-2-ylamino)-benzamide |
| 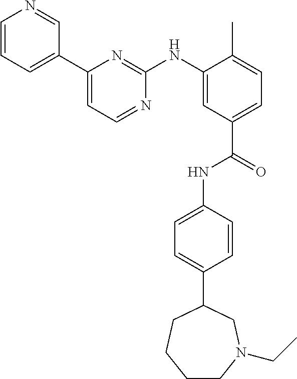 [SMILES: CCN1CCCCC(c2ccc(NC(=O)c3ccc(C)c(Nc4nccc(-c5cccnc5)n4)c3)cc2)C1] | N-[4-(1-Ethyl-azepan-3-yl)-phenyl]-4-methyl-3-(4-pyridin-3-yl- pyrimidin-2-ylamino)-benzamide |
| 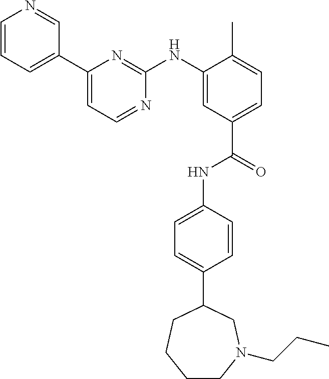 [SMILES: CCCN1CCCCC(c2ccc(NC(=O)c3ccc(C)c(Nc4nccc(-c5cccnc5)n4)c3)cc2)C1] | 4-Methyl-N-[4-(1-propyl-azepan-3-yl)-phenyl]-3-(4-pyridin-3- yl-pyrimidin-2-ylamino)-benzamide |
| 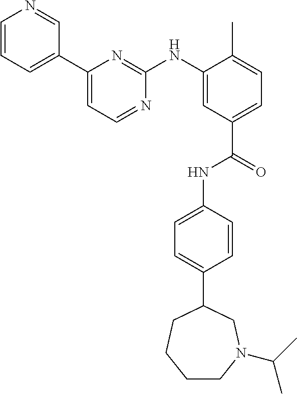 [SMILES: Cc1ccc(C(=O)Nc2ccc(C3CCCCN(C(C)C)C3)cc2)cc1Nc1nccc(-c2cccnc2)n1] | N-[4-(1-Isopropyl-azepan-3-yl)-phenyl]-4-methyl-3-(4-pyridin- 3-yl-pyrimidin-2-ylamino)-benzamide |
| 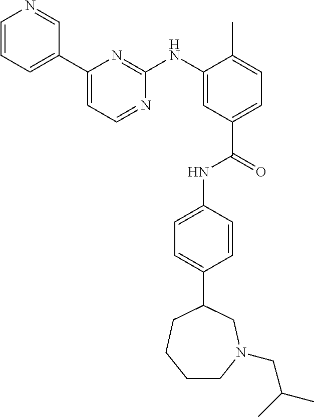 [SMILES: Cc1ccc(C(=O)Nc2ccc(C3CCCCN(CC(C)C)C3)cc2)cc1Nc1nccc(-c2cccnc2)n1] | N-[4-(1-Isobutyl-azepan-3-yl)-phenyl]-4-methyl-3-(4-pyridin-3- yl-pyrimidin-2-ylamino)-benzamide |
| 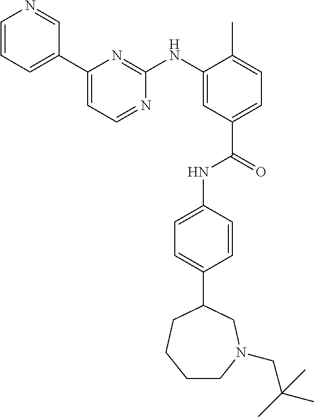 [SMILES: Cc1ccc(C(=O)Nc2ccc(C3CCCCN(CC(C)(C)C)C3)cc2)cc1Nc1nccc(-c2cccnc2)n1] | N-{4-[1-(2,2-Dimethyl-propyl)-azepan-3-yl]-phenyl}-4-methyl- 3-(4-pyridin-3-yl-pyrimidin-2-ylamino)-benzamide |
| 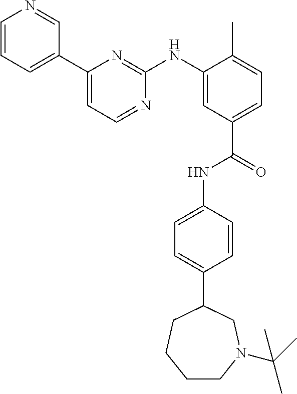 [SMILES: Cc1ccc(C(=O)Nc2ccc(C3CCCCN(C(C)(C)C)C3)cc2)cc1Nc1nccc(-c2cccnc2)n1] | N-[4-(1-tert-Butyl-azepan-3-yl)-phenyl]-4-methyl-3-(4-pyridin- 3-yl-pyrimidin-2-ylamino)-benzamide |
| 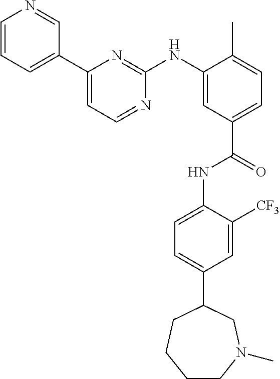 [SMILES: Cc1ccc(C(=O)Nc2ccc(C3CCCCN(C)C3)cc2C(F)(F)F)cc1Nc1nccc(-c2cccnc2)n1] | 4-Methyl-N-[4-(1-methyl-azepan-3-yl)-2-trifluoromethyl- phenyl]-3-(4-pyridin-3-yl-pyrimidin-2-ylamino)-benzamide |
| 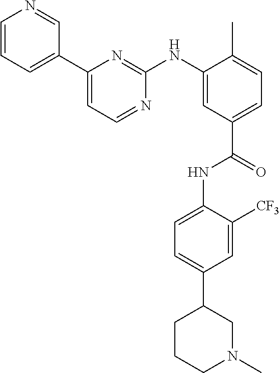 [SMILES: Cc1ccc(C(=O)Nc2ccc(C3CCCN(C)C3)cc2C(F)(F)F)cc1Nc1nccc(-c2cccnc2)n1] | 4-Methyl-N-[4-(1-methyl-piperidin-3-yl)-2-trifluoromethyl- phenyl]-3-(4-pyridin-3-yl-pyrimidn-2-ylamino)-benzamide |
| 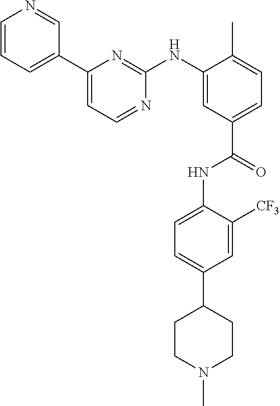 [SMILES: Cc1ccc(C(=O)Nc2ccc(C3CCN(C)CC3)cc2C(F)(F)F)cc1Nc1nccc(-c2cccnc2)n1] | 4-Methyl-N-[4-(1-methyl-piperidin-4-yl)-2-trifluoromethyl- phenyl]-3-(4-pyridin-3-yl-pyrimidin-2-ylamino)-benzamide |
| 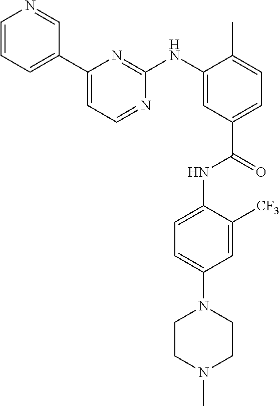 [SMILES: Cc1ccc(C(=O)Nc2ccc(N3CCN(C)CC3)cc2C(F)(F)F)cc1Nc1nccc(-c2cccnc2)n1] | 4-Methyl-N-[4-(4-methyl-piperazin-1-yl)-2-trifluoromethyl- phenyl]-3-(4-pyridin-3-yl-pyrimidin-2-ylamino)-benzamide |
| 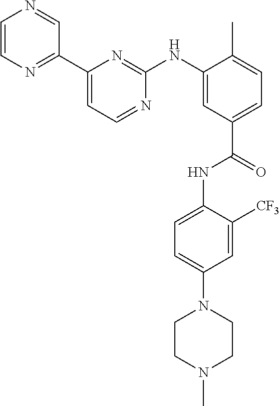 [SMILES: Cc1ccc(C(=O)Nc2ccc(N3CCN(C)CC3)cc2C(F)(F)F)cc1Nc1nccc(-c2cnccn2)n1] | 4-Methyl-N-[4-(4-methyl-piperazin-1-yl)-2-trifluoromethyl- phenyl]-3-(4-pyrazin-2-yl-pyrimidin-2-ylamino)-benzamide |
| 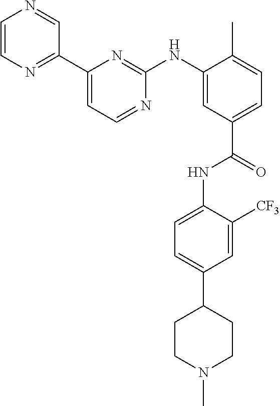 [SMILES: Cc1ccc(C(=O)Nc2ccc(C3CCN(C)CC3)cc2C(F)(F)F)cc1Nc1nccc(-c2cnccn2)n1] | 4-Methyl-N-[4-(1-methyl-piperidin-4-yl)-2-trifluoromethyl- phenyl]-3-(4-pyrazin-2-yl-pyrimidin-2-ylamino)-benzamide |
| 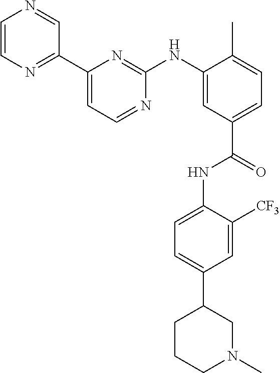 [SMILES: Cc1ccc(C(=O)Nc2ccc(C3CCCN(C)C3)cc2C(F)(F)F)cc1Nc1nccc(-c2cnccn2)n1] | 4-Methyl-N-[4-(1-methyl-piperidin-3-yl)-2-trifluoromethyl- phenyl]-3-(4-pyrazin-2-yl-pyrimidin-2-ylamino)-benzamide |
| 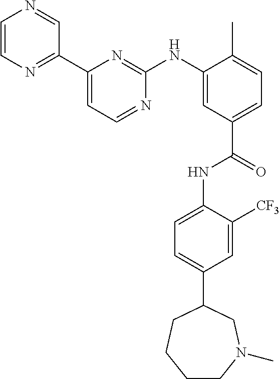 [SMILES: Cc1ccc(C(=O)Nc2ccc(C3CCCCN(C)C3)cc2C(F)(F)F)cc1Nc1nccc(-c2cnccn2)n1] | 4-Methyl-N-[4-(1-methyl-azepan-3-yl)-2-trifluoromethyl- phenyl]-3-(4-pyrazin-2-yl-pyrimidin-2-ylamino)-benzamide |
| 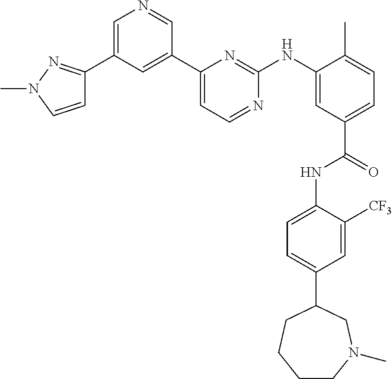 | 4-Methyl-N-[4-(1-methyl-azepan-3-yl)-2-trifluoromethyl- phenyl]-3-{4-[5-(1-methyl-1H-pyrazol-3-yl)-pyridin-3-yl]- pyrimidin-2-ylamino}-benzamide |
| 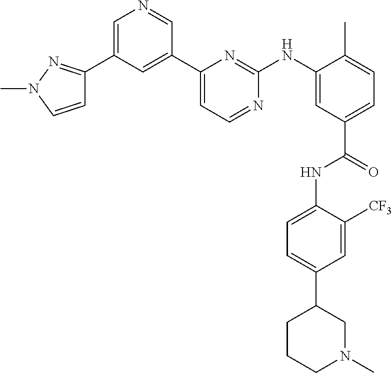 [SMILES: Cc1ccc(C(=O)Nc2ccc(C3CCCN(C)C3)cc2C(F)(F)F)cc1Nc1nccc(-c2cncc(-c3ccn(C)n3)c2)n1] | 4-Methyl-N-[4-(1-methyl-piperidin-3-yl)-2-trifluoromethyl- phenyl]-3-{4-[5-(1-methyl-1H-pyrazol-3-yl)-pyridin-3-yl]- pyrimidin-2-yalmino}-benzamide |
| 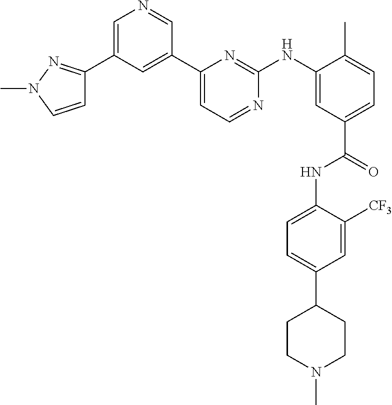 [SMILES: Cc1ccc(C(=O)Nc2ccc(C3CCN(C)CC3)cc2C(F)(F)F)cc1Nc1nccc(-c2cncc(-c3ccn(C)n3)c2)n1] | 4-Methyl-N-[4-(1-methyl-piperidin-4-yl)-2-trifluoromethyl- phenyl]-3-{4-[5-(1-methyl-1H-pyrazol-3-yl)-pyridin-3-yl]- pyrimidin-2-ylamino}-benzamide |
| 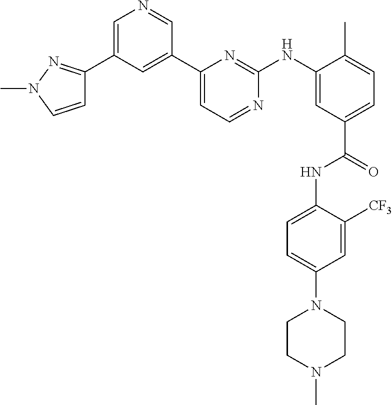 [SMILES: Cc1ccc(C(=O)Nc2ccc(N3CCN(C)CC3)cc2C(F)(F)F)cc1Nc1nccc(-c2cncc(-c3ccn(C)n3)c2)n1] | 4-Methyl-N-[4-(4-methyl-piperazin-1-yl)-2-trifluoromethyl- phenyl]-3-{4-[5-(1-methyl-1H-pyrazol-3-yl)-pyridin-3-yl]- pyrimidin-2-ylamino}-benzamide |
| 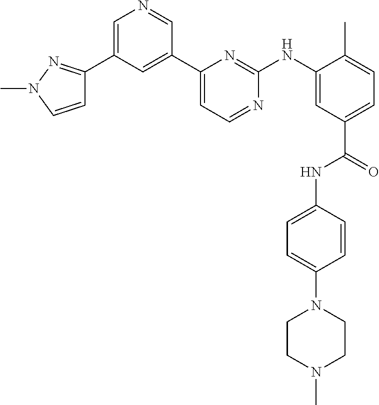 [SMILES: Cc1ccc(C(=O)Nc2ccc(N3CCN(C)CC3)cc2)cc1Nc1nccc(-c2cncc(-c3ccn(C)n3)c2)n1] | 4-Methyl-N-[4-(4-methyl-piperazin-1-yl)-phenyl]-3-{4-[5-(1- methyl-1H-pyrazol-3-yl)-pyridin-3-yl]-pyrimidin-2-ylamino}- benzamide |
| 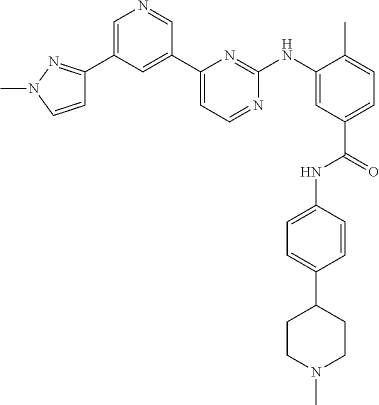 [SMILES: Cc1ccc(C(=O)Nc2ccc(C3CCN(C)CC3)cc2)cc1Nc1nccc(-c2cncc(-c3ccn(C)n3)c2)n1] | 4-Methyl-N-[4-(1-methyl-piperidin-4-yl)-phenyl]-3-{4-[5-(1- methyl-1H-pyrazol-3-yl)-pyridin-3-yl]-pyrimidin-2-ylamino}- benzamide |
| 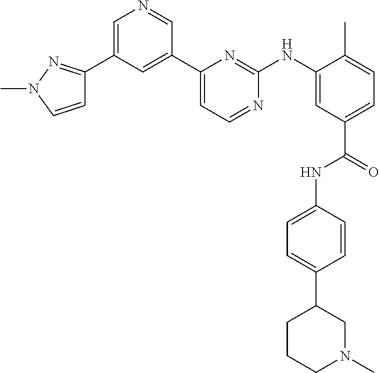 | 4-Methyl-N-[4-(1-methyl-piperidin-3-yl)-phenyl]-3-{4-[5-(1- methyl-1H-pyrazol-3-yl)-pyridin-3-yl]-pyrimidin-2-ylamino}- benzamide |
| 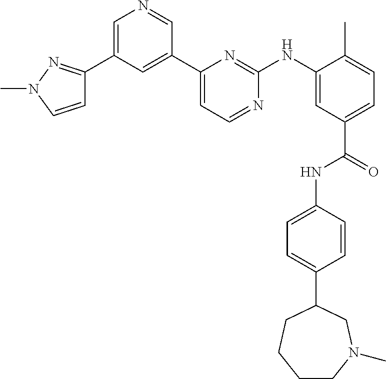 | 4-Methyl-N-[4-(1-methyl-azepan-3-yl)-phenyl]-3-{4-[5-(1- methyl-1H-pyrazol-3-yl)-pyridin-3-yl]-pyrimidin-2-ylamino}- benzamide |
| 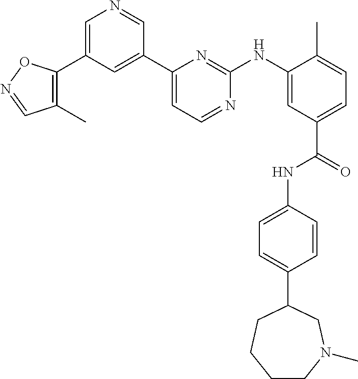 [SMILES: Cc1ccc(C(=O)Nc2ccc(C3CCCCN(C)C3)cc2)cc1Nc1nccc(-c2cncc(-c3oncc3C)c2)n1] | 4-Methyl-N-[4-(1-methyl-azepan-3-yl)-phenyl]-3-{4-[5-(4- methyl-isoxazol-5-yl)-pyridin-3-yl]-pyrimidin-2-ylamino}- benzamide |
| 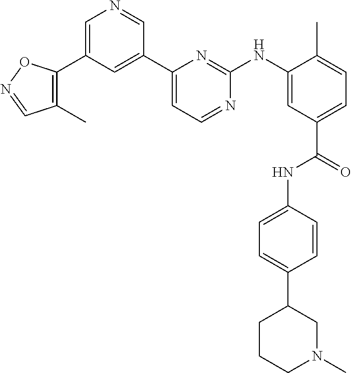 [SMILES: Cc1ccc(C(=O)Nc2ccc(C3CCCN(C)C3)cc2)cc1Nc1nccc(-c2cncc(-c3oncc3C)c2)n1] | 4-Methyl-3-{4-[5-(4-methyl-isoxazol-5-yl)-pyridin-3-yl]- pyrimidin-2-ylamino}-N-[4-(1-methyl-piperidin-3-yl)-phenyl]- benzamide |
| 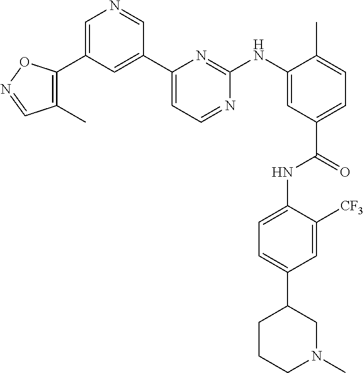 [SMILES: Cc1ccc(C(=O)Nc2ccc(C3CCCN(C)C3)cc2C(F)(F)F)cc1Nc1nccc(-c2cncc(-c3oncc3C)c2)n1] | 4-Methyl-3-{4-[5-(4-methyl-isoxazol-5-yl)-pyridin-3-yl]- pyrimidin-2-ylamino}-N-[4-(1-methyl-piperidin-3-yl)-2- trifluoromethyl-phenyl]-benzamide |
| 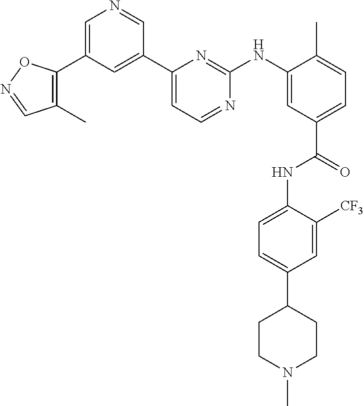 [SMILES: Cc1ccc(C(=O)Nc2ccc(C3CCN(C)CC3)cc2C(F)(F)F)cc1Nc1nccc(-c2cncc(-c3oncc3C)c2)n1] | 4-Methyl-3-{4-[5-(4-methyl-isoxazol-5-yl)-pyridin-3-yl]- pyrimidin-2-ylamino}-N-[4-(1-methyl-piperidin-4-yl)-2- trifluoromethyl-phenyl]-benzamide |
| 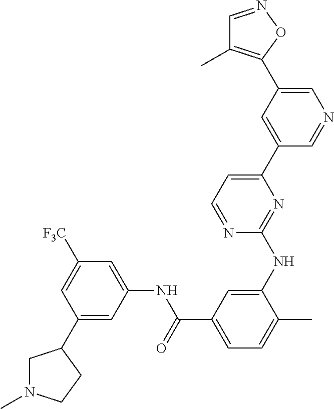 [SMILES: Cc1ccc(C(=O)Nc2cc(C3CCN(C)C3)cc(C(F)(F)F)c2)cc1Nc1nccc(-c2cncc(-c3oncc3C)c2)n1] | 4-Methyl-3-{4-[5-(4-methyl-isoxazol-5-yl)-pyridin-3-yl]- pyrimidin-2-ylamino}-N-[3-(1-methyl-pyrrolidin-3-yl)-5- trifluoromethyl-phenyl]-benzamide |
| 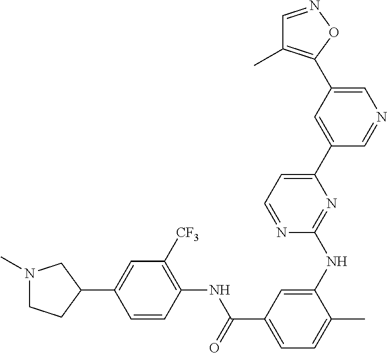 [SMILES: Cc1ccc(C(=O)Nc2ccc(C3CCN(C)C3)cc2C(F)(F)F)cc1Nc1nccc(-c2cncc(-c3oncc3C)c2)n1] | 4-Methyl-3-{4-[5-(4-methyl-isoxazol-5-yl)-pyridin-3-yl]- pyrimidin-2-ylamino}-N-[4-(1-methyl-pyrrolidin-3-yl)-2- trifluoromethyl-phenyl]-benzamide |
| 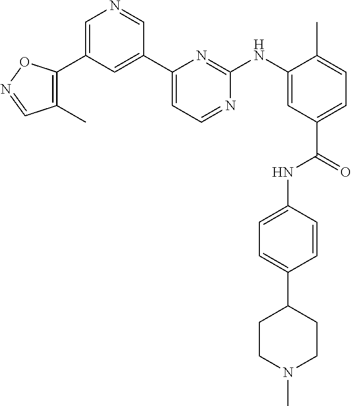 [SMILES: Cc1ccc(C(=O)Nc2ccc(C3CCN(C)CC3)cc2)cc1Nc1nccc(-c2cncc(-c3oncc3C)c2)n1] | 4-Methyl-3-{4-[5-(4-methyl-isoxazol-5-yl)-pyridin-3-yl]- pyrimidin-2-ylamino}-N-[4-(1-methyl-piperidin-4-yl)-phenyl]- benzamide |
| 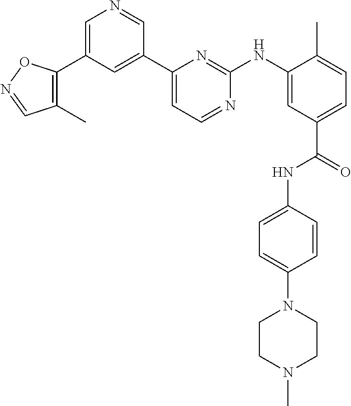 [SMILES: Cc1ccc(C(=O)Nc2ccc(N3CCN(C)CC3)cc2)cc1Nc1nccc(-c2cncc(-c3oncc3C)c2)n1] | 4-Methyl-3-{4-[5-(4-methyl-isoxazol-5-yl)-pyridin-3-yl]- pyrimidin-2-ylamino}-N-[4-(4-methyl-piperazin-1-yl)-phenyl]- benzamide |
| 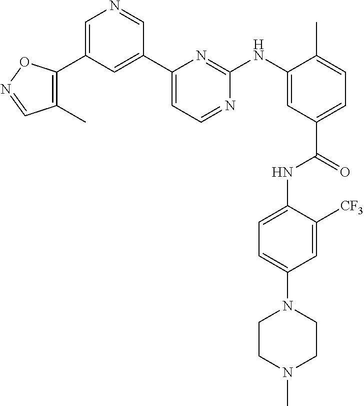 [SMILES: Cc1ccc(C(=O)Nc2ccc(N3CCN(C)CC3)cc2C(F)(F)F)cc1Nc1nccc(-c2cncc(-c3oncc3C)c2)n1] | 4-Methyl-3-{4-[5-(4-methyl-isoxazol-5-yl)-pyridin-3-yl]- pyrimidin-2-ylamino}-N-[4-(4-methyl-piperazin-1-yl)-2- trifluoromethyl-phenyl]-benzamide |
| 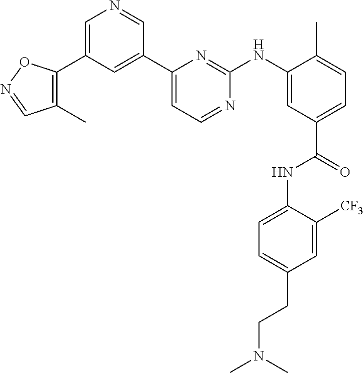 [SMILES: Cc1ccc(C(=O)Nc2ccc(CCN(C)C)cc2C(F)(F)F)cc1Nc1nccc(-c2cncc(-c3oncc3C)c2)n1] | N-[4-(2-Dimethylamino-ethyl)-2-trifluoromethyl-phenyl]-4- methyl-3-{4-[5-(4-methyl-isoxazol-5-yl)-pyridin-3-yl]- pyrimidin-2-ylamino}-benzamide |
| 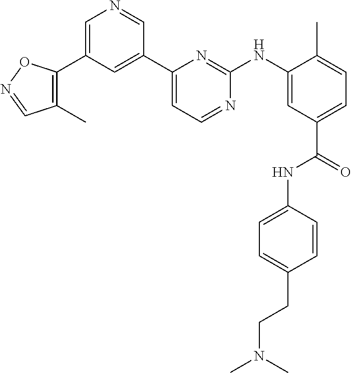 [SMILES: Cc1ccc(C(=O)Nc2ccc(CCN(C)C)cc2)cc1Nc1nccc(-c2cncc(-c3oncc3C)c2)n1] | N-[4-(2-Dimethylamino-ethyl)-phenyl]-4-methyl-3-{4-[5-(4- methyl-isoxazol-5-yl)-pyridin-3-yl]-pyrimidin-2-ylamino}- benzamide |
| 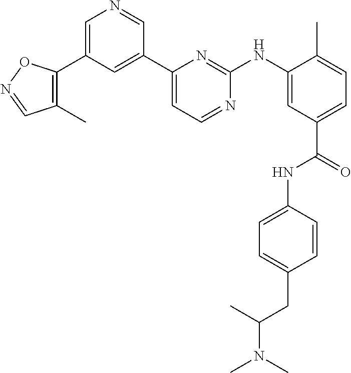 [SMILES: Cc1ccc(C(=O)Nc2ccc(CC(C)N(C)C)cc2)cc1Nc1nccc(-c2cncc(-c3oncc3C)c2)n1] | N-[4-(2-Dimethylamino-propyl)-phenyl]-4-methyl-3-{4-[5-(4- methyl-isoxazol-5-yl)-pyridin-3-yl]-pyrimidin-2-ylamino}- benzamide |
| 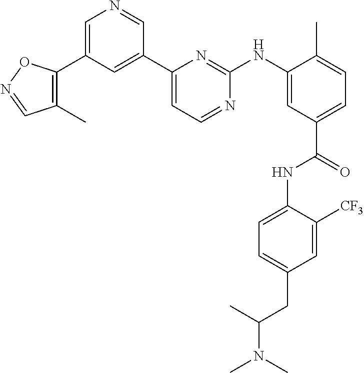 [SMILES: Cc1ccc(C(=O)Nc2ccc(CC(C)N(C)C)cc2C(F)(F)F)cc1Nc1nccc(-c2cncc(-c3oncc3C)c2)n1] | N-[4-(2-Dimethylamino-propyl)-2-trifluoromethyl-phenyl]-4- methyl-3-{4-[5-(4-methyl-isoxazol-5-yl)-pyridin-3-yl]- pyrimidin-2-ylamino}-benzamide |
| 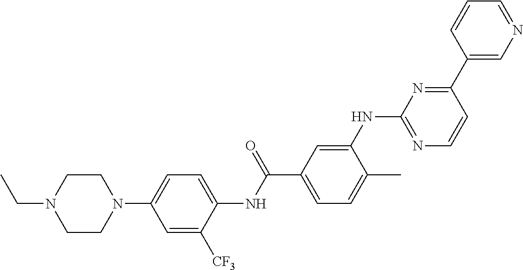 [SMILES: CCN1CCN(c2ccc(NC(=O)c3ccc(C)c(Nc4nccc(-c5cccnc5)n4)c3)c(C(F)(F)F)c2)CC1] | N-[4-(4-Ethyl-piperazin-1-yl)-2-trifluoromethyl-phenyl]-4- methyl-3-(4-pyridin-3-yl-pyrimidin-2-ylamino)-benzamide |
| 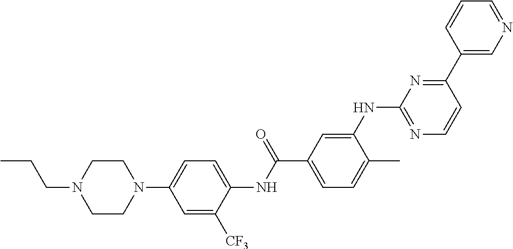 [SMILES: CCCN1CCN(c2ccc(NC(=O)c3ccc(C)c(Nc4nccc(-c5cccnc5)n4)c3)c(C(F)(F)F)c2)CC1] | 4-Methyl-N-[4-(4-propyl-piperazin-1-yl)-2-trifluoromethyl- phenyl]-3-(4-pyridin-3-yl-pyrimidin-2-ylamino)-benzamide |
| 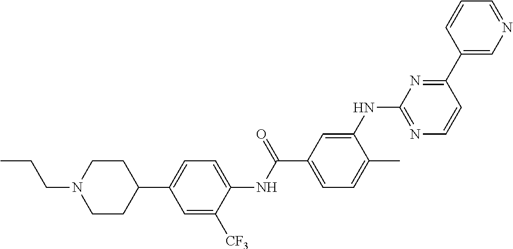 [SMILES: CCCN1CCC(c2ccc(NC(=O)c3ccc(C)c(Nc4nccc(-c5cccnc5)n4)c3)c(C(F)(F)F)c2)CC1] | 4-Methyl-N-[4-(1-propyl-piperidin-4-yl)-2-trifluoromethyl- phenyl]-3-(4-pyridin-3-yl-pyrimidin-2-ylamino)-benzamide |
| 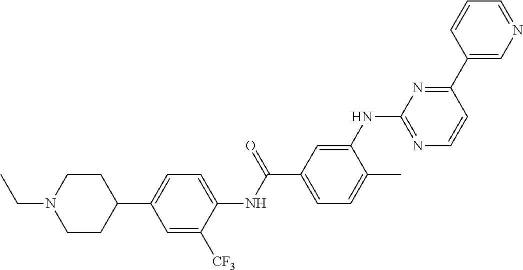 [SMILES: CCN1CCC(c2ccc(NC(=O)c3ccc(C)c(Nc4nccc(-c5cccnc5)n4)c3)c(C(F)(F)F)c2)CC1] | N-[4-(1-Ethyl-piperidin-4-yl)-2-trifluoromethyl-phenyl]-4- methyl-3-(4-pyridin-3-yl-pyrimidin-2-ylamino)-benzamide |
| 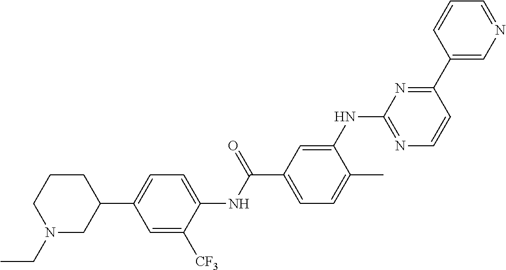 [SMILES: CCN1CCCC(c2ccc(NC(=O)c3ccc(C)c(Nc4nccc(-c5cccnc5)n4)c3)c(C(F)(F)F)c2)C1] | N-[4-(1-Ethyl-piperidin-3-yl)-2-trifluoromethyl-phenyl]-4- methyl-3-(4-pyridin-3-yl-pyrimidin-2-ylamino)-benzamide |
| 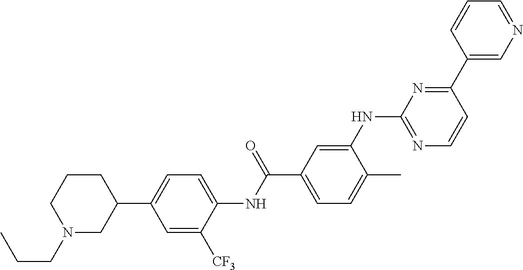 [SMILES: CCCN1CCCC(c2ccc(NC(=O)c3ccc(C)c(Nc4nccc(-c5cccnc5)n4)c3)c(C(F)(F)F)c2)C1] | 4-Methyl-N-[4-(1-propyl-piperidin-3-yl)-2-trifluoromethyl- phenyl]-3-(4-pyridin-3-yl-pyrimidin-2-ylamino)-benzamide |
| 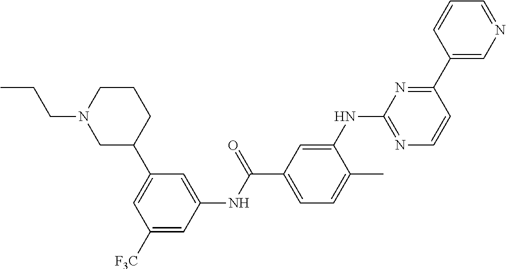 [SMILES: CCCN1CCCC(c2cc(NC(=O)c3ccc(C)c(Nc4nccc(-c5cccnc5)n4)c3)cc(C(F)(F)F)c2)C1] | 4-Methyl-N-[3-(1-propyl-piperidin-3-yl)-5-trifluoromethyl- phenyl]-3-(4-pyridin-3-yl-pyrimidin-2-ylamino)-benzamide |
| 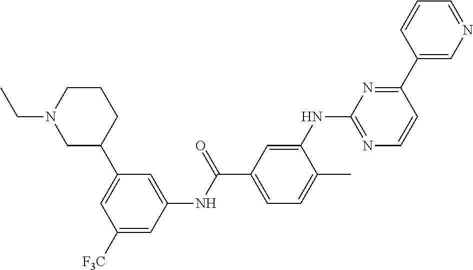 [SMILES: CCN1CCCC(c2cc(NC(=O)c3ccc(C)c(Nc4nccc(-c5cccnc5)n4)c3)cc(C(F)(F)F)c2)C1] | N-[3-(1-Ethyl-piperidin-3-yl)-5-trifluoromethyl-phenyl]-4- methyl-3-(4-pyridin-3-yl-pyrimidin-2-ylamino)-benzamide |
| 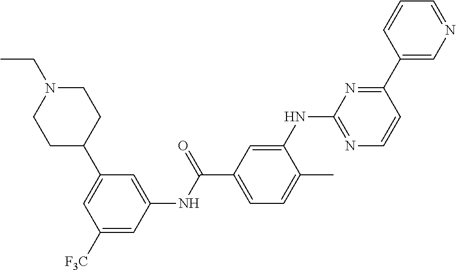 [SMILES: CCN1CCC(c2cc(NC(=O)c3ccc(C)c(Nc4nccc(-c5cccnc5)n4)c3)cc(C(F)(F)F)c2)CC1] | N-[3-(1-Ethyl-piperidin-4-yl)-5-trifluoromethyl-phenyl]-4- methyl-3-(4-pyridin-3-yl-pyrimidin-2-ylamino)-benzamide |
| 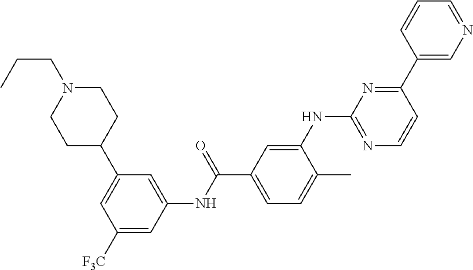 [SMILES: CCCN1CCC(c2cc(NC(=O)c3ccc(C)c(Nc4nccc(-c5cccnc5)n4)c3)cc(C(F)(F)F)c2)CC1] | 4-Methyl-N-[3-(1-propyl-piperidin-4-yl)-5-trifluoromethyl- phenyl]-3-(4-pyridin-3-yl-pyrimidin-2-ylamino)-benzamide |
| 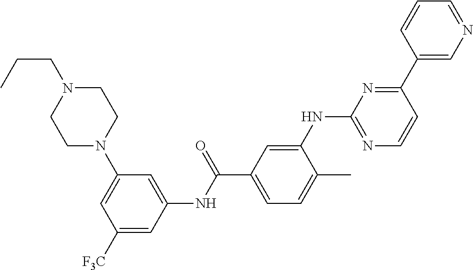 [SMILES: CCCN1CCN(c2cc(NC(=O)c3ccc(C)c(Nc4nccc(-c5cccnc5)n4)c3)cc(C(F)(F)F)c2)CC1] | 4-Methyl-N-[3-(4-propyl-piperazin-1-yl)-5-trifluoromethyl- phenyl]-3-(4-pyridin-3-yl-pyrimidin-2-ylamino)-benzamide |
| 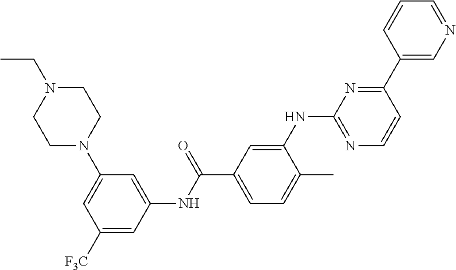 [SMILES: CCN1CCN(c2cc(NC(=O)c3ccc(C)c(Nc4nccc(-c5cccnc5)n4)c3)cc(C(F)(F)F)c2)CC1] | N-[3-(4-Ethyl-piperazin-1-yl)-5-trifluoromethyl-phenyl]-4- methyl-3-(4-pyridin-3-yl-pyrimidin-2-ylamino)-benzamide |
| 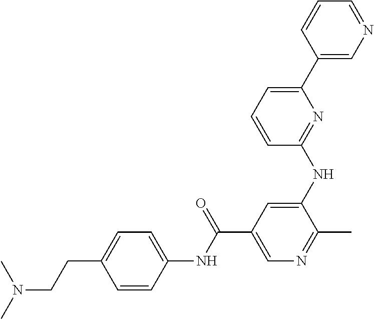 [SMILES: Cc1ncc(C(=O)Nc2ccc(CCN(C)C)cc2)cc1Nc1cccc(-c2cccnc2)n1] | N-[4-(2-Dimethylamino-ethyl)-phenyl]-6-methyl-5-(4-pyridin- 3-yl-pyrimidin-2-ylamino)-nicotinamide |
| 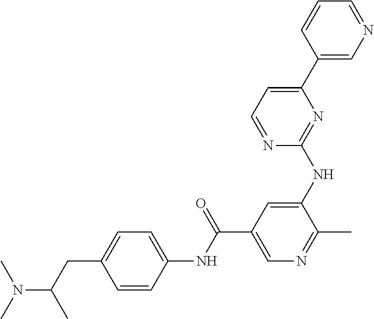 [SMILES: Cc1ncc(C(=O)Nc2ccc(CC(C)N(C)C)cc2)cc1Nc1nccc(-c2cccnc2)n1] | N-[4-(2-Dimethylamino-propyl)-phenyl]-6-methyl-5-(4-pyridin- 3-yl-pyrimidin-2-ylamino)-nicotinamide |
| 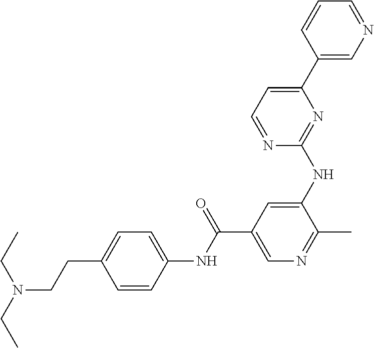 [SMILES: CCN(CC)CCc1ccc(NC(=O)c2cnc(C)c(Nc3nccc(-c4cccnc4)n3)c2)cc1] | N-[4-(2-Diethylamino-ethyl)-phenyl]-6-methyl-5-(4-pyridin-3- yl-pyrimidin-2-ylamino)-nicotinamide |
| 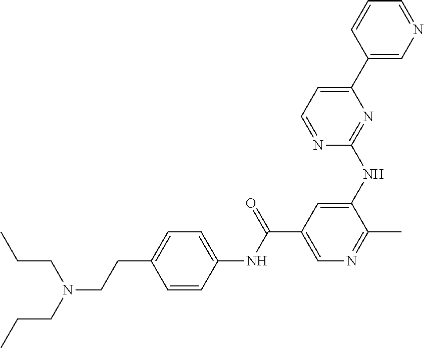 [SMILES: CCCN(CCC)CCc1ccc(NC(=O)c2cnc(C)c(Nc3nccc(-c4cccnc4)n3)c2)cc1] | N-[4-(2-Dipropylamino-ethyl)-phenyl]-6-methyl-5-(4-pyridin-3- yl-pyrimidin-2-ylamino)-nicotinamide |
| 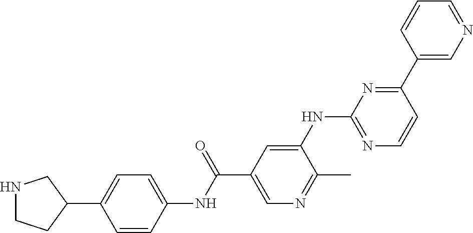 [SMILES: Cc1ncc(C(=O)Nc2ccc(C3CCNC3)cc2)cc1Nc1nccc(-c2cccnc2)n1] | 6-Methyl-5-(4-pyridin-3-yl-pyrimidin-2-ylamino)-N-(4- pyrrolidin-3-yl-phenyl)-nicotinamide |
| 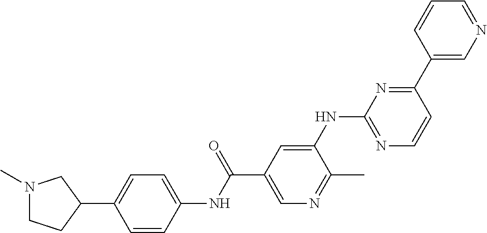 [SMILES: Cc1ncc(C(=O)Nc2ccc(C3CCN(C)C3)cc2)cc1Nc1nccc(-c2cccnc2)n1] | 6-Methyl-N-[4-(1-methyl-pyrrolidin-3-yl)-phenyl]-5-(4-pyridin- 3-yl-pyrimidin-2-ylamino)-nicotinamide |
| 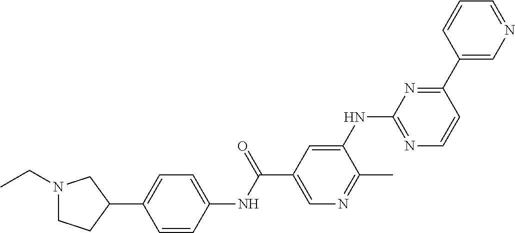 [SMILES: CCN1CCC(c2ccc(NC(=O)c3cnc(C)c(Nc4nccc(-c5cccnc5)n4)c3)cc2)C1] | N-[4-(1-Ethyl-pyrrolidin-3-yl)-phenyl]-6-methyl-5-(4-pyridin-3- yl-pyrimidin-2-ylamino)-nicotinamide |
| 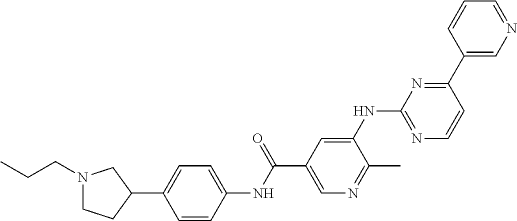 [SMILES: CCCN1CCC(c2ccc(NC(=O)c3cnc(C)c(Nc4nccc(-c5cccnc5)n4)c3)cc2)C1] | 6-Methyl-N-[4-(1-propyl-pyrrolidin-3-yl)-phenyl]-5-(4-pyridin- 3-yl-pyrimidin-2-ylamino)-nicotinamide |
| 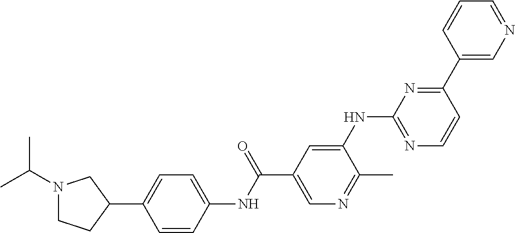 [SMILES: Cc1ncc(C(=O)Nc2ccc(C3CCN(C(C)C)C3)cc2)cc1Nc1nccc(-c2cccnc2)n1] | N-[4-(1-Isopropyl-pyrrolidin-3-yl)-phenyl]-6-methyl-5-(4- pyridin-3-yl-pyrimidin-2-ylamino)-nicotinamide |
| 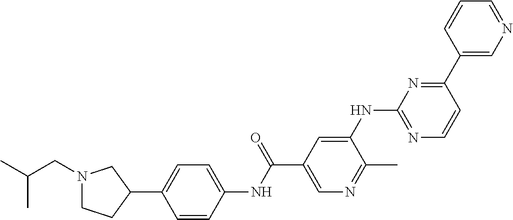 [SMILES: Cc1ncc(C(=O)Nc2ccc(C3CCN(CC(C)C)C3)cc2)cc1Nc1nccc(-c2cccnc2)n1] | N-[4-(1-Isobutyl-pyrrolidin-3-yl)-phenyl]-6-methyl-5-(4- pyridin-3-yl-pyrimidin-2-ylamino)-nicotinamide |
| 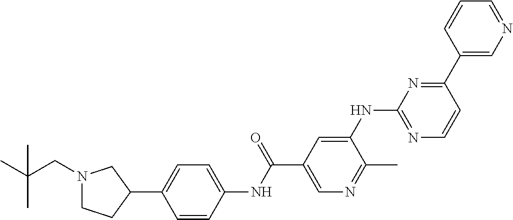 [SMILES: Cc1ncc(C(=O)Nc2ccc(C3CCN(CC(C)(C)C)C3)cc2)cc1Nc1nccc(-c2cccnc2)n1] | N-{4-[1-(2,2-Dimethyl-propyl)-pyrrolidin-3-yl]-phenyl}-6- methyl-5-(4-pyridin-3-yl-pyrimidin-2-ylamino)-nicotinamide |
| 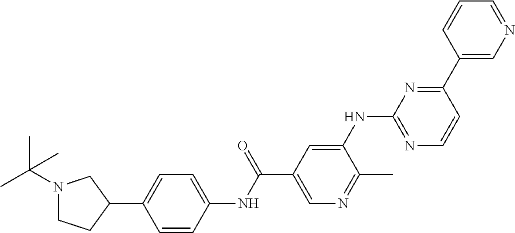 [SMILES: Cc1ncc(C(=O)Nc2ccc(C3CCN(C(C)(C)C)C3)cc2)cc1Nc1nccc(-c2cccnc2)n1] | N-[4-(1-tert-Butyl-pyrrolidin-3-yl)-phenyl]-6-methyl-5-(4- pyridin-3-yl-pyrimidin-2-ylamino)-nicotinamide |
| 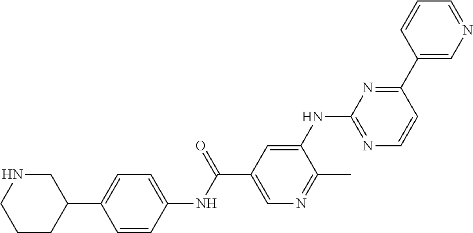 [SMILES: Cc1ncc(C(=O)Nc2ccc(C3CCCNC3)cc2)cc1Nc1nccc(-c2cccnc2)n1] | 6-Methyl-N-(4-piperidin-3-yl-phenyl)-5-(4-pyridin-3-yl- pyrimidin-2-ylamino)-nicotinamdie |
| 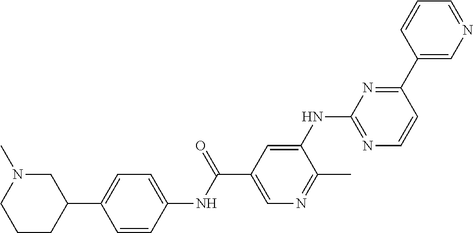 [SMILES: Cc1ncc(C(=O)Nc2ccc(C3CCCN(C)C3)cc2)cc1Nc1nccc(-c2cccnc2)n1] | 6-Methyl-N-[4-(1-methyl-piperidin-3-yl)-phenyl]-5-(4-pyridin- 3-yl-pyrimidin-2-ylamino)-nicotinamide |
| 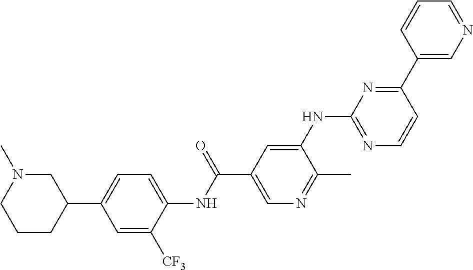 [SMILES: Cc1ncc(C(=O)Nc2ccc(C3CCCN(C)C3)cc2C(F)(F)F)cc1Nc1nccc(-c2cccnc2)n1] | 6-Methyl-N-[4-(1-methyl-piperidin-3-yl)-2-trifluoromethyl- phenyl]-5-(4-pyridin-3-yl-pyrimidin-2-ylamino)-nicotinamide |
| 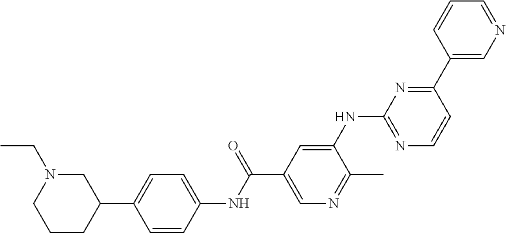 [SMILES: CCN1CCCC(c2ccc(NC(=O)c3cnc(C)c(Nc4nccc(-c5cccnc5)n4)c3)cc2)C1] | N-[4-(1-Ethyl-piperidin-3-yl)-phenyl]-6-methyl-5-(4-pyridin-3- yl-pyrimidin-2-ylamino)-nicotinamide |
| 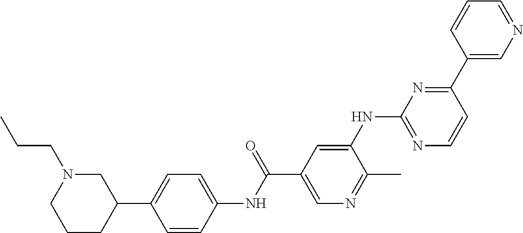 [SMILES: CCCN1CCCC(c2ccc(NC(=O)c3cnc(C)c(Nc4nccc(-c5cccnc5)n4)c3)cc2)C1] | 6-Methyl-N-[4-(1-propyl-piperidin-3-yl)-phenyl]-5-(4-pyridin- 3-yl-pyrimidin-2-ylamino)-nicotinamide |
| 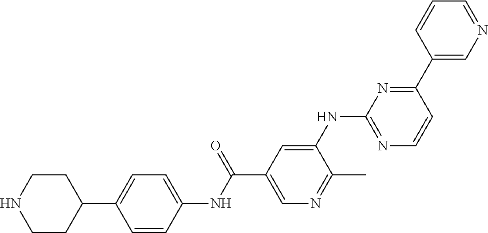 [SMILES: Cc1ncc(C(=O)Nc2ccc(C3CCNCC3)cc2)cc1Nc1nccc(-c2cccnc2)n1] | 6-Methyl-N-(4-piperidin-4-yl-phenyl)-5-(4-pyridin-3-yl- pyrimidin-2-ylamino)-nicotinamide |
| 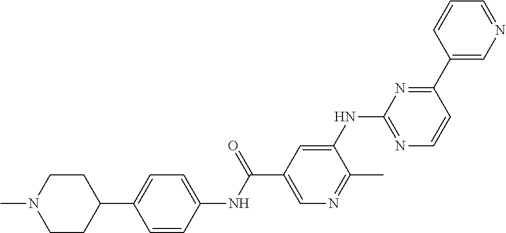 [SMILES: Cc1ncc(C(=O)Nc2ccc(C3CCN(C)CC3)cc2)cc1Nc1nccc(-c2cccnc2)n1] | 6-Methyl-N-[4-(1-methyl-piperidin-4-yl)-phenyl]-5-(4-pyridin- 3-yl-pyrimidin-2-ylamino)-nicotinamide |
| 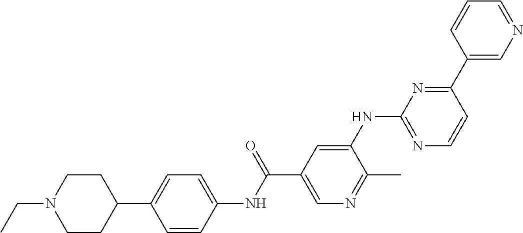 [SMILES: CCN1CCC(c2ccc(NC(=O)c3cnc(C)c(Nc4nccc(-c5cccnc5)n4)c3)cc2)CC1] | N-[4-(1-Ethyl-piperidin-4-yl)-phenyl]-6-methyl-5-(4-pyridin-3- yl-pyrimidin-2-ylamino)-nicotinamide |
| 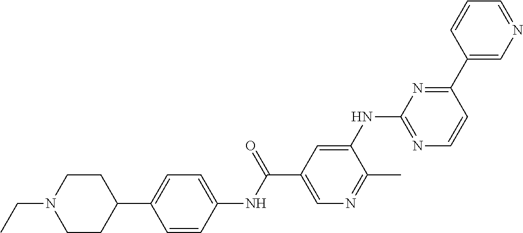 [SMILES: CCN1CCC(c2ccc(NC(=O)c3cnc(C)c(Nc4nccc(-c5cccnc5)n4)c3)cc2)CC1] | 6-Methyl-N-[4-(1-propyl-piperidin-4-yl)-phenyl]-5-(4-pyridin- 3-yl-pyrimidin-2-ylamino)-nicotinamide |
| 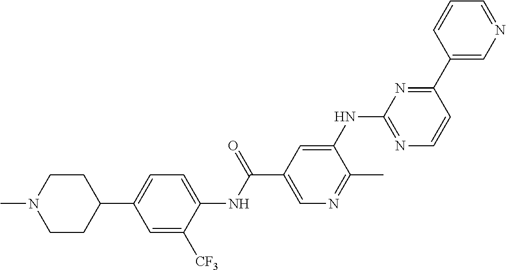 [SMILES: Cc1ncc(C(=O)Nc2ccc(C3CCN(C)CC3)cc2C(F)(F)F)cc1Nc1nccc(-c2cccnc2)n1] | 6-Methyl-N-[4-(1-methyl-piperidin-4-yl)-2-trifluoromethyl- phenyl]-5-(4-pyridin-3-yl-pyrimidin-2-ylamnio)-nicotinamide |
| 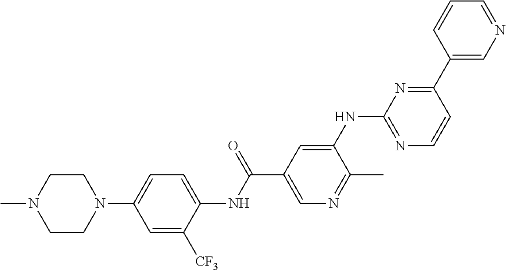 [SMILES: Cc1ncc(C(=O)Nc2ccc(N3CCN(C)CC3)cc2C(F)(F)F)cc1Nc1nccc(-c2cccnc2)n1] | 6-Methyl-N-[4-(4-methyl-piperazin-1-yl)-2-trifluoromethyl- phenyl]-5-(4-pyridin-3-yl-pyrimidin-2-ylamino)-nicotinamide |
| 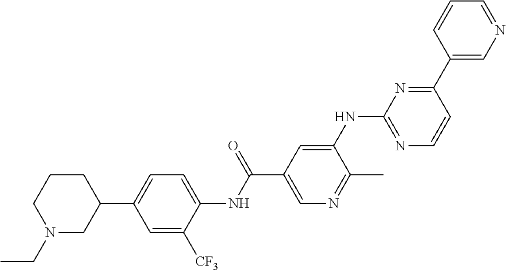 [SMILES: CCN1CCCC(c2ccc(NC(=O)c3cnc(C)c(Nc4nccc(-c5cccnc5)n4)c3)c(C(F)(F)F)c2)C1] | N-[4-(1-Ethyl-piperidin-3-yl)-2-trifluoromethyl-phenyl]-6- methyl-5-(4-pyridin-3-yl-pyrimidin-2-ylamino)-nicotinamide |
| 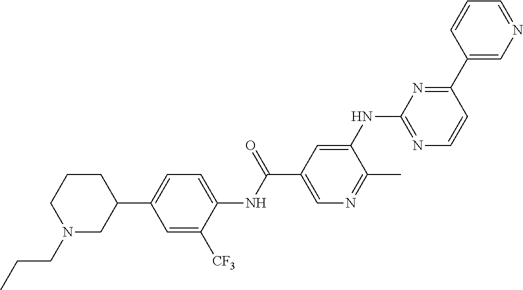 [SMILES: CCCN1CCCC(c2ccc(NC(=O)c3cnc(C)c(Nc4nccc(-c5cccnc5)n4)c3)c(C(F)(F)F)c2)C1] | 6-Methyl-N-[4-(1-propyl-piperidin-3-yl)-2-fluoromethyl- phenyl]-5-(4-pyridin-3-yl-pyrimidin-2-ylamino)-nicotinamide |
| 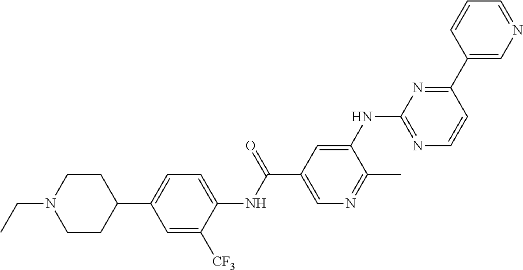 [SMILES: CCN1CCC(c2ccc(NC(=O)c3cnc(C)c(Nc4nccc(-c5cccnc5)n4)c3)c(C(F)(F)F)c2)CC1] | N-[4-(1-Ethyl-piperidin-4-yl)-2-fluoromethyl-phenyl]-6- methyl-5-(4-pyridin-3-yl-pyrimidin-2-ylamino)-nicotinamide |
| 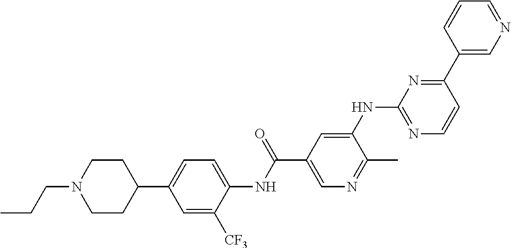 [SMILES: CCCN1CCC(c2ccc(NC(=O)c3cnc(C)c(Nc4nccc(-c5cccnc5)n4)c3)c(C(F)(F)F)c2)CC1] | 6-Methyl-N-[4-(1-propyl-piperidin-4-yl)-2-trifluoromethyl- phenyl]-5-(4-pyridin-3-yl-pyrimidin-2-ylamino)-nicotinamide |
| 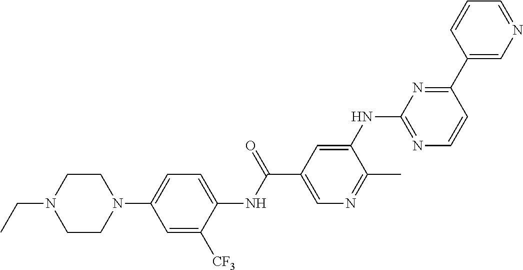 [SMILES: CCN1CCN(c2ccc(NC(=O)c3cnc(C)c(Nc4nccc(-c5cccnc5)n4)c3)c(C(F)(F)F)c2)CC1] | N-[4-(4-Ethyl-piperazin-1-yl)-2-trifluoromethyl-phenyl]-6- methyl-5-(4-pyridin-3-yl-pyrimidin-2-ylamino)-nicotinamide |
| 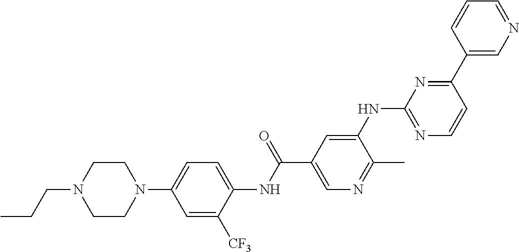 [SMILES: CCCN1CCN(c2ccc(NC(=O)c3cnc(C)c(Nc4nccc(-c5cccnc5)n4)c3)c(C(F)(F)F)c2)CC1] | 6-Methyl-N-[4-(4-propyl-piperazin-1-yl)-2-trifluoromethyl- phenyl]-5-(4-pyridin-3-yl-pyrimidin-2-ylamino)-nicotinamide |
| 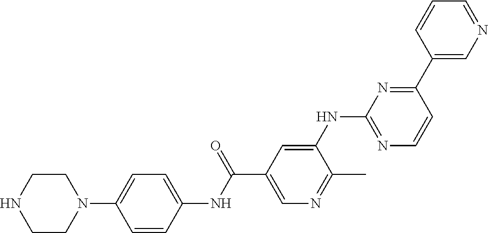 [SMILES: Cc1ncc(C(=O)Nc2ccc(N3CCNCC3)cc2)cc1Nc1nccc(-c2cccnc2)n1] | 6-Methyl-N-(4-piperazin-1-yl-phenyl)-5-(4-pyridin-3-yl- pyrimidin-2-ylamino)-nicotinamide |
| 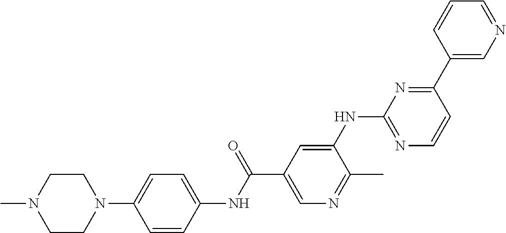 [SMILES: Cc1ncc(C(=O)Nc2ccc(N3CCN(C)CC3)cc2)cc1Nc1nccc(-c2cccnc2)n1] | 6-Methyl-N-[4-(4-methyl-piperazin-1-yl)-phenyl]-5-(4-pyridin- 3-yl-pyrimidin-2-ylamino)-nicotinamide |
| 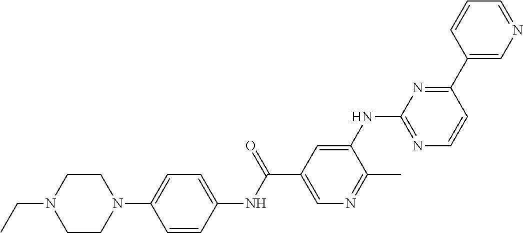 [SMILES: CCN1CCN(c2ccc(NC(=O)c3cnc(C)c(Nc4nccc(-c5cccnc5)n4)c3)cc2)CC1] | N-[4-(4-Ethyl-piperazin-1-yl)-phenyl]-6-methyl-5-(4-pyridin-3- yl-pyrimidin-2-ylamino)-nicotinamide |
| 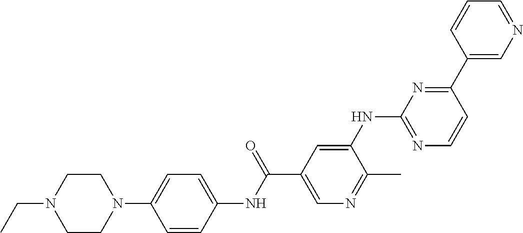 [SMILES: CCN1CCN(c2ccc(NC(=O)c3cnc(C)c(Nc4nccc(-c5cccnc5)n4)c3)cc2)CC1] | 6-Methyl-N-[4-(4-propyl-piperazin-1-yl)-phenyl]-5-(4-pyridin- 3-yl-pyrimidin-2-ylamino)-nicotinamide |
| 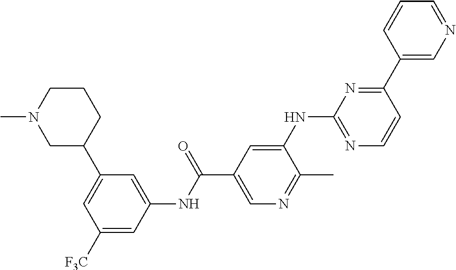 [SMILES: Cc1ncc(C(=O)Nc2cc(C3CCCN(C)C3)cc(C(F)(F)F)c2)cc1Nc1nccc(-c2cccnc2)n1] | 6-Methyl-N-[3-(1-methyl-piperidin-3-yl)-5-trifluoromethyl- phenyl]-5-(4-pyridin-3-yl-pyrimidin-2-ylamino)-nicotinamide |
| 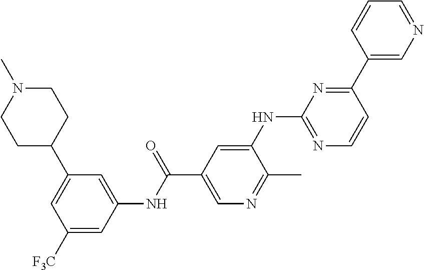 [SMILES: Cc1ncc(C(=O)Nc2cc(C3CCN(C)CC3)cc(C(F)(F)F)c2)cc1Nc1nccc(-c2cccnc2)n1] | 6-Methyl-N-[3-(1-methyl-piperidin-4-yl)-5-trifluoromethyl- phenyl]-5-(4-pyridin-3-yl-pyrimidin-2-ylamino)-nicotinamide |
| 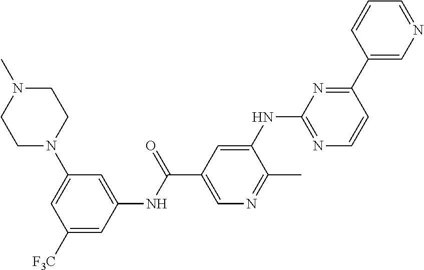 [SMILES: Cc1ncc(C(=O)Nc2cc(N3CCN(C)CC3)cc(C(F)(F)F)c2)cc1Nc1nccc(-c2cccnc2)n1] | 6-Methyl-N-[3-(4-methyl-piperazin-1-yl)-5-trifluoromethyl- phenyl]-5-(4-pyridin-3-yl-pyrimidin-2-ylamino)-nicotinamide |
| 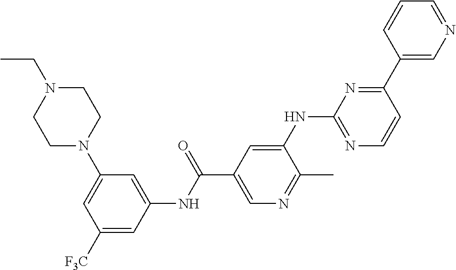 [SMILES: CCN1CCN(c2cc(NC(=O)c3cnc(C)c(Nc4nccc(-c5cccnc5)n4)c3)cc(C(F)(F)F)c2)CC1] | N-[3-(4-Ethyl-piperazin-1-yl)-5-trifluoromethyl-phenyl]-6- methyl-5-(4-pyridin-3-yl-pyrimidin-2-ylamino)-nicotinamide |
| 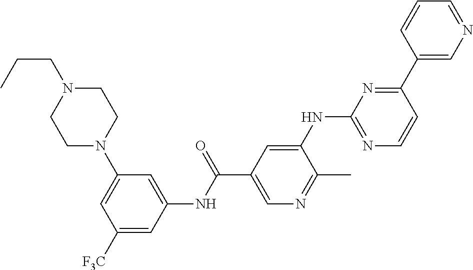 [SMILES: CCCN1CCN(c2cc(NC(=O)c3cnc(C)c(Nc4nccc(-c5cccnc5)n4)c3)cc(C(F)(F)F)c2)CC1] | 6-Methyl-N-[3-(4-propyl-piperazin-1-yl)-5-trifluoromethyl- phenyl]-5-(4-pyridin-3-yl-pyrimidin-2-ylamino)-nicotinamide |
| 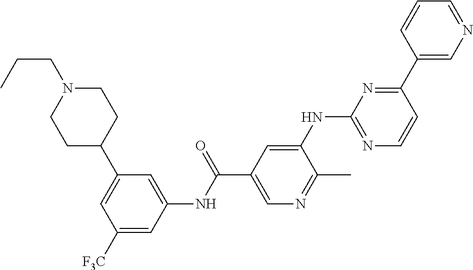 [SMILES: CCCN1CCC(c2cc(NC(=O)c3cnc(C)c(Nc4nccc(-c5cccnc5)n4)c3)cc(C(F)(F)F)c2)CC1] | 6-Methyl-N-[3-(1-propyl-piperidin-4-yl)-5-trifluoromethyl- phenyl]-5-(4-pyridin-3-yl-pyrimidin-2-ylamino)-nicotinamide |
| 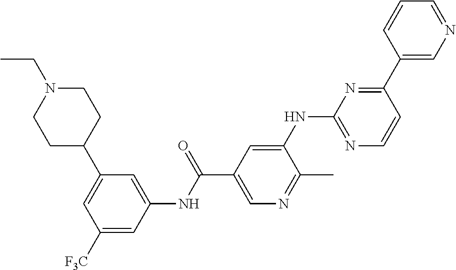 [SMILES: CCN1CCC(c2cc(NC(=O)c3cnc(C)c(Nc4nccc(-c5cccnc5)n4)c3)cc(C(F)(F)F)c2)CC1] | N-[3-(1-Ethyl-piperidin-4-yl)-5-trifluoromethyl-phenyl]-6- methyl-5-(4-pyridin-3-yl-pyrimidin-2-ylamino)-nicotinamide |
| 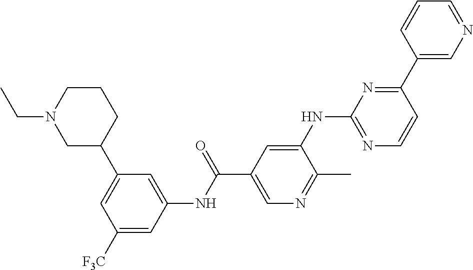 [SMILES: CCN1CCCC(c2cc(NC(=O)c3cnc(C)c(Nc4nccc(-c5cccnc5)n4)c3)cc(C(F)(F)F)c2)C1] | N-[3-(1-Ethyl-piperidin-3-yl)-5-trifluoromethyl-phenyl]-6- methyl-5-(4-pyridin-3-yl-pyrimidin-2-ylamino)-nicotinamide |
| 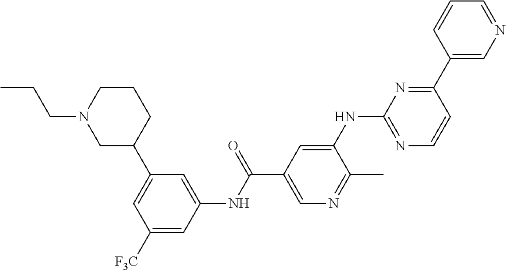 [SMILES: CCCN1CCCC(c2cc(NC(=O)c3cnc(C)c(Nc4nccc(-c5cccnc5)n4)c3)cc(C(F)(F)F)c2)C1] | 6-Methyl-N-[3-(1-propyl-piperidin-3-yl)-5-trifluoromethyl- phenyl]-5-(4-pyridin-3-yl-pyrimidin-2-ylamino)-nicotinamide |

In some embodiments, the compounds of the present invention are represented in Formula (IV), or a pharmaceutically acceptable salt, solvate, hydrate, cocrystal, or prodrug thereof, wherein X₁, X₂, or X₃ is independently CH or N; Y is CH or N; R₈ or R₉ independently is hydrogen or the masking group defined above; R₄ is the same as defined in Formula (I). In an embodiment, R₄ is hydrogen or CF₃.

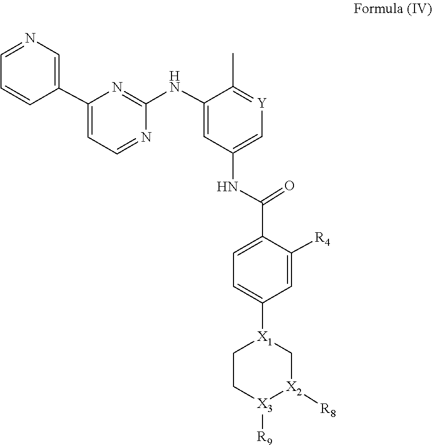

In an embodiment, R₈ or R₉ is independently selected from the group consisting of H,

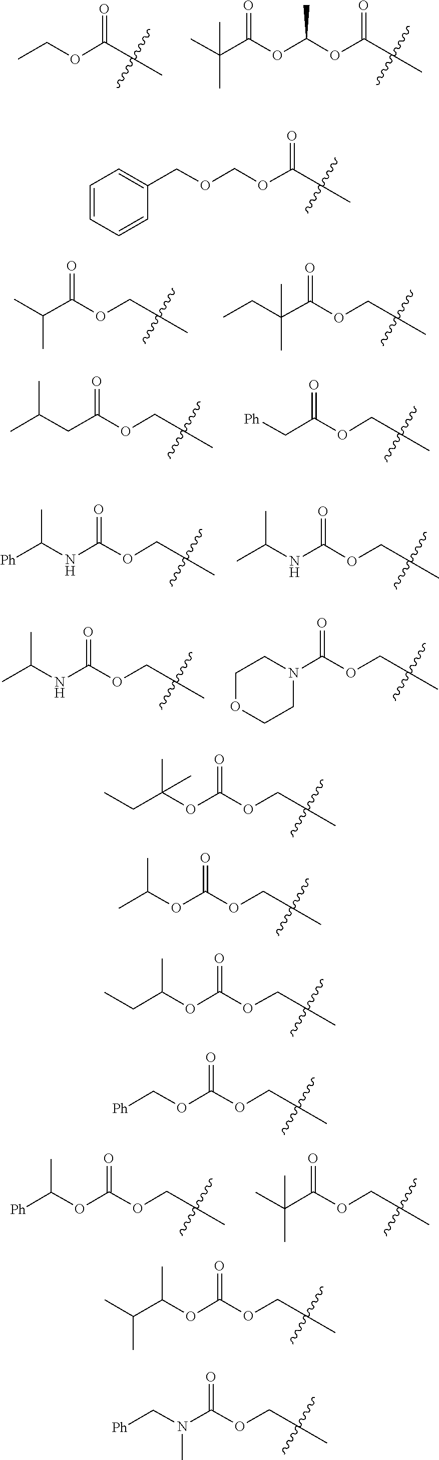

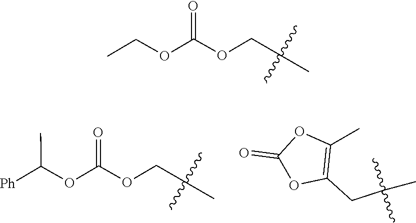

and combinations thereof.

Examples of compounds of Formula (IV) are listed in Table 3.

TABLE 3
Embodiments of Formula (IV)

| | |
|---|---|
| 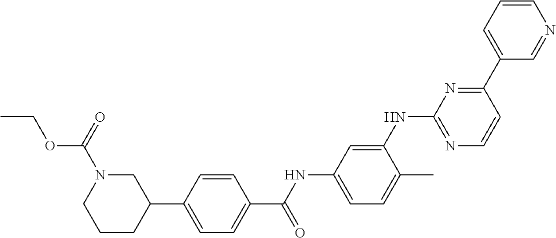 [SMILES: CCOC(=O)N1CCCC(c2ccc(C(=O)Nc3ccc(C)c(Nc4nccc(-c5cccnc5)n4)c3)cc2)C1] | 3-{4-[4-Methyl-3-(4-pyridin-3-yl-pyrimidin-2- ylamino)-phenylcarbamoyl]-phenyl}-piperidine-1- carboxylic acid ethyl ester |
| 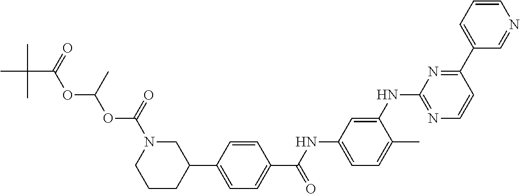 [SMILES: Cc1ccc(NC(=O)c2ccc(C3CCCN(C(=O)OC(C)OC(=O)C(C)(C)C)C3)cc2)cc1Nc1nccc(-c2cccnc2)n1] | 3-{4-[4-Methyl-3-(4-pyridin-3-yl-pyrimidin-2- ylamino)-phenylcarbamoyl]-phenyl}-piperidine-1- carboxylic acid 1-(2,2-dimethyl-propionyloxy)-ethyl ester |
| 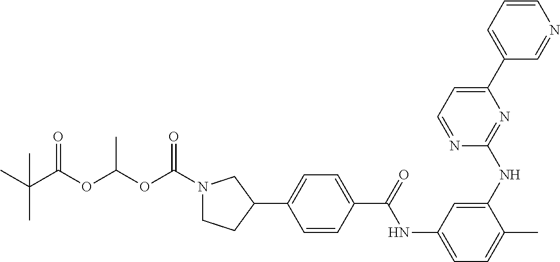 [SMILES: Cc1ccc(NC(=O)c2ccc(C3CCN(C(=O)OC(C)OC(=O)C(C)(C)C)C3)cc2)cc1Nc1nccc(-c2cccnc2)n1] | 3-{4-[4-Methyl-3-(4-pyridin-3-yl-pyrimidin-2- ylamino)-phenylcarbamoyl]-phenyl}-pyrrolidine-1- carboxylic acid 1-(2,2-dimethyl-propionyloxy)-ethyl ester |
| 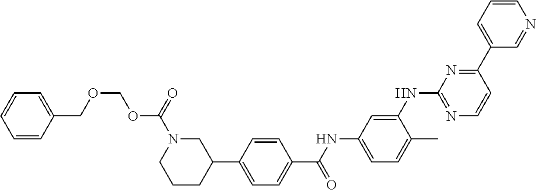 [SMILES: Cc1ccc(NC(=O)c2ccc(C3CCCN(C(=O)OCOCc4ccccc4)C3)cc2)cc1Nc1nccc(-c2cccnc2)n1] | 3-{4-[4-Methyl-3-(4-pyridin-3-yl-pyrimidin-2- ylamino)-phenylcarbamoyl]-phenyl}-piperidine-1- carboxylic acid benzyloxymethyl ester |
| 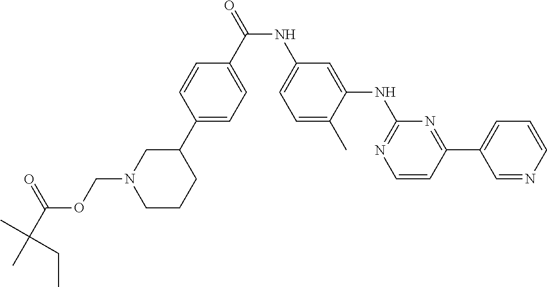 [SMILES: CCC(C)(C)C(=O)OCN1CCCC(c2ccc(C(=O)Nc3ccc(C)c(Nc4nccc(-c5cccnc5)n4)c3)cc2)C1] | 2,2-Dimethyl-butyric acid 3-{4-[4-methyl-3-(4- pyridin-3-yl-pyrimidin-2-ylamino)-phenyl- carbamoyl]-phenyl}-piperidin-1-ylmethyl ester |
| 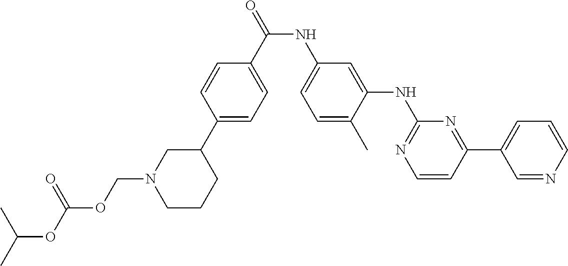 [SMILES: Cc1ccc(NC(=O)c2ccc(C3CCCN(COC(=O)OC(C)C)C3)cc2)cc1Nc1nccc(-c2cccnc2)n1] | Carbonic acid isopropyl ester 3-{4-[4-methyl-3-(4- pyridin-3-yl-pyrimidin-2-ylamino)-phenylcarbamoyl]- phenyl}-piperidin-1-ylmethyl ester |
| 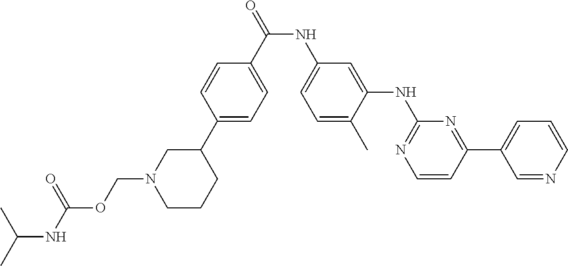 [SMILES: Cc1ccc(NC(=O)c2ccc(C3CCCN(COC(=O)NC(C)C)C3)cc2)cc1Nc1nccc(-c2cccnc2)n1] | Isopropyl-carbamic acid 3-{4-[4-methyl-3-(4-pyridin- 3-yl-pyrimidin-2-ylamino)-phenylcarbamoyl]- phenyl}-piperidin-1-ylmethyl ester |
| 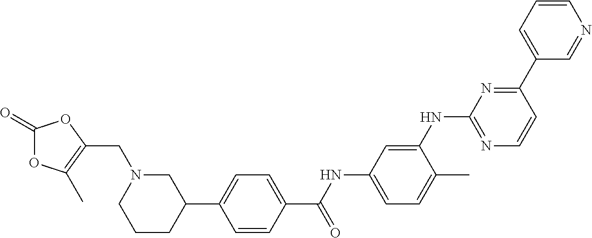 [SMILES: Cc1ccc(NC(=O)c2ccc(C3CCCN(Cc4oc(=O)oc4C)C3)cc2)cc1Nc1nccc(-c2cccnc2)n1] | 4-[1-(5-Methyl-2-oxo-[1,3]dioxol-4-ylmethyl)- piperidin-3-yl]-N-[4-methyl-3-(4-pyridin-3-yl- pyrimidin-2-ylamino)-phenyl]-benzamide |
| 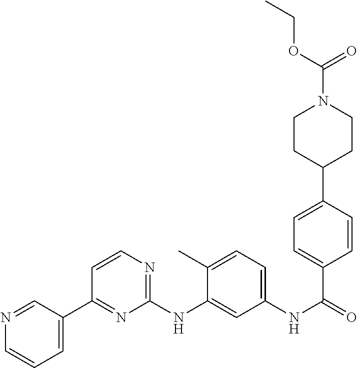 [SMILES: CCOC(=O)N1CCC(c2ccc(C(=O)Nc3ccc(C)c(Nc4nccc(-c5cccnc5)n4)c3)cc2)CC1] | 4-{4-[4-Methyl-3-(4-pyridin-3-yl-pyrimidin-2- ylamino)-phenylcarbamoyl]-phenyl}-piperidine-1- carboxylic acid ethyl ester |
| 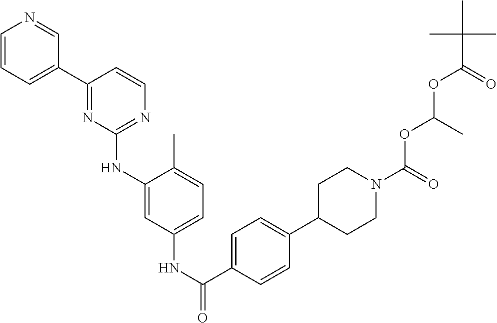 [SMILES: Cc1ccc(NC(=O)c2ccc(C3CCN(C(=O)OC(C)OC(=O)C(C)(C)C)CC3)cc2)cc1Nc1nccc(-c2cccnc2)n1] | 4-{4-[4-Methyl-3-(4-pyridin-3-yl-pyrimidin-2- ylamino)-phenylcarbamoyl]-phenyl}-piperidine-1- carboxylic acid 1-(2,2-dimethyl-propionyloxy)-ethyl ester |
| 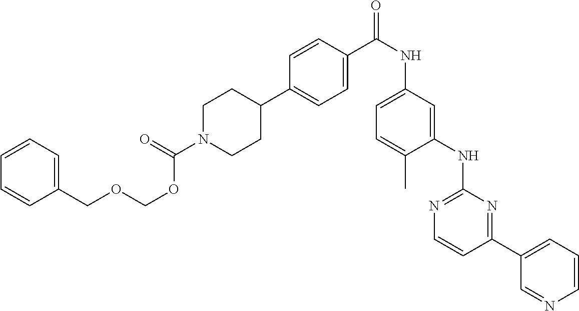 [SMILES: Cc1ccc(NC(=O)c2ccc(C3CCN(C(=O)OCOCc4ccccc4)CC3)cc2)cc1Nc1nccc(-c2cccnc2)n1] | 4-{4-[4-Methyl-3-(4-pyridin-3-yl-pyrimidin-2- ylamino)-phenylcarbamoyl]-phenyl}-piperidine-1- carboxylic acid benzyloxymethyl ester |
| 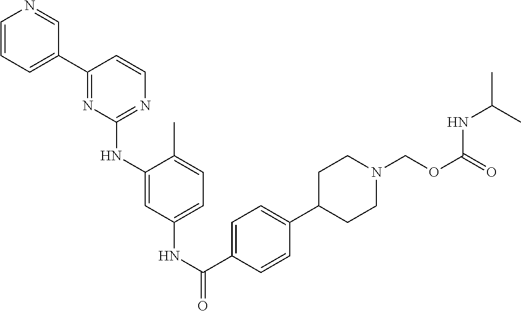 [SMILES: Cc1ccc(NC(=O)c2ccc(C3CCN(COC(=O)NC(C)C)CC3)cc2)cc1Nc1nccc(-c2cccnc2)n1] | Isopropyl-carbamic acid 4-{4-[4-methyl-3-(4-pyridin- 3-yl-pyrimidin-2-ylamino)-phenylcarbamoyl]- phenyl}-piperidin-1-ylmethyl ester |
| 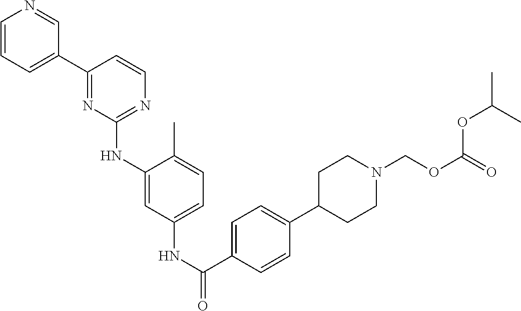 [SMILES: Cc1ccc(NC(=O)c2ccc(C3CCN(COC(=O)OC(C)C)CC3)cc2)cc1Nc1nccc(-c2cccnc2)n1] | Carbonic acid isopropyl ester 4-{4-[4-methyl-3-(4- pyridin-3-yl-pyrimidin-2-ylamino)-phenyl- carbamoyl]-phenyl}-piperidin-1-ylmethyl ester |
| 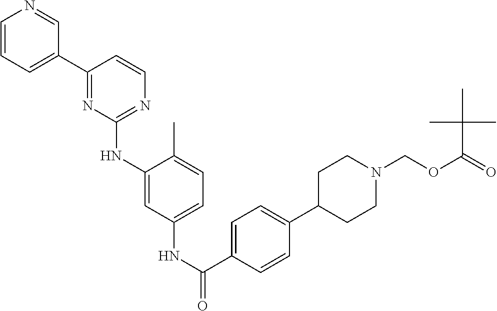 [SMILES: Cc1ccc(NC(=O)c2ccc(C3CCN(COC(=O)C(C)(C)C)CC3)cc2)cc1Nc1nccc(-c2cccnc2)n1] | 2,2-Dimethyl-propionic acid 4-{4-[4-methyl-3-(4- pyridin-3-yl-pyrimidin-2-ylamino)-phenyl- carbamoyl]-phenyl}-piperidin-1-ylmethyl ester |
| 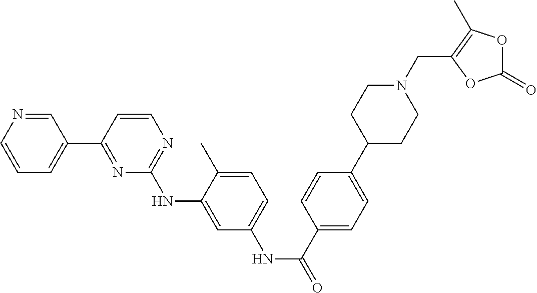 [SMILES: Cc1ccc(NC(=O)c2ccc(C3CCN(Cc4oc(=O)oc4C)CC3)cc2)cc1Nc1nccc(-c2cccnc2)n1] | 4-[1-(5-Methyl-2-oxo-[1,3]dioxol-4-ylmethyl)- piperidin-4-yl]-N-[4-methyl-3-(4-pyridin-3-yl- pyrimidin-2-ylamino)-phenyl]-benzamide |
| 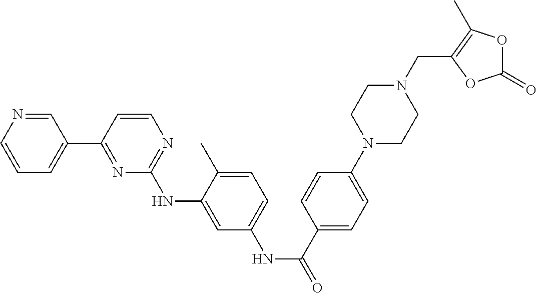 [SMILES: Cc1ccc(NC(=O)c2ccc(N3CCN(Cc4oc(=O)oc4C)CC3)cc2)cc1Nc1nccc(-c2cccnc2)n1] | 4-[4-(5-Methyl-2-oxo-[1,3]dioxol-4-ylmethyl)- piperazin-1-yl]-N-[4-methyl-3-(4-pyridin-3-yl- pyrimidin-2-ylamino)-phenyl]-benzamide |
| 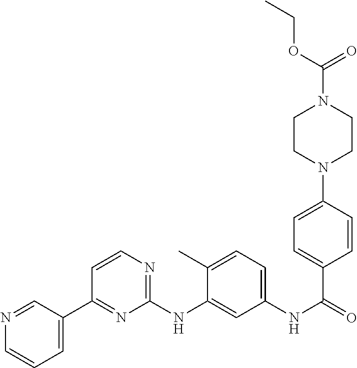 [SMILES: CCOC(=O)N1CCN(c2ccc(C(=O)Nc3ccc(C)c(Nc4nccc(-c5cccnc5)n4)c3)cc2)CC1] | 4-{4-[4-Methyl-3-(4-pyridin-3-yl-pyrimidin-2- ylamino)-phenylcarbamoyl]-phenyl}-piperazine-1- carboxylic acid ethyl ester |
| 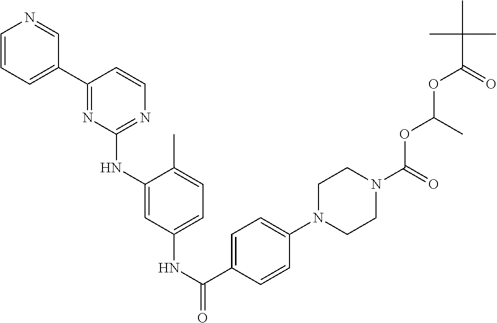 [SMILES: Cc1ccc(NC(=O)c2ccc(N3CCN(C(=O)OC(C)OC(=O)C(C)(C)C)CC3)cc2)cc1Nc1nccc(-c2cccnc2)n1] | 4-{4-[4-Methyl-3-(4-pyridin-3-yl-pyrimidin-2- ylamino)-phenylcarbamoyl]-phenyl}-piperazine-1- carboxylic acid 1-(2,2-dimethyl-propionyloxy)-ethyl ester |
| 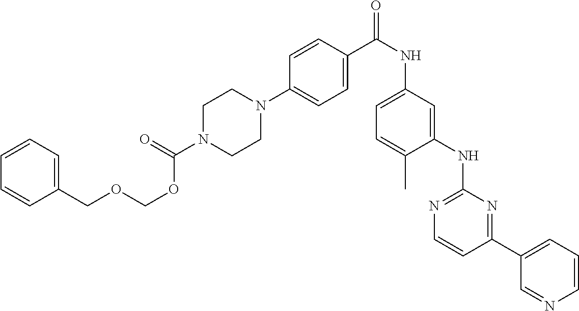 [SMILES: Cc1ccc(NC(=O)c2ccc(N3CCN(C(=O)OCOCc4ccccc4)CC3)cc2)cc1Nc1nccc(-c2cccnc2)n1] | 4-{4-[4-Methyl-3-(4-pyridin-3-yl-pyrimidin-2- ylamino)-phenylcarbamoyl]-phenyl}-piperazine-1- carboxylic acid benzyloxymethyl ester |
| 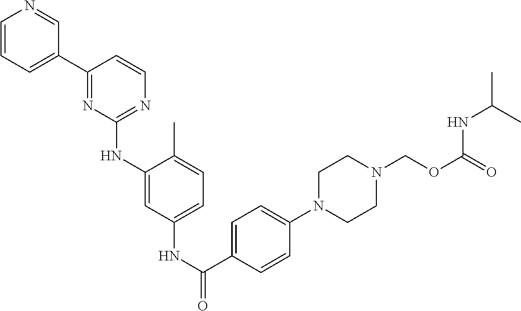 [SMILES: Cc1ccc(NC(=O)c2ccc(N3CCN(COC(=O)NC(C)C)CC3)cc2)cc1Nc1nccc(-c2cccnc2)n1] | Isopropyl-carbamic acid 4-{4-[4-methyl-3-(4- pyridin-3-yl-pyrimidin-2-ylamino)-phenyl- carbamoyl]-phenyl}-piperazin-1-ylmethyl ester |
| 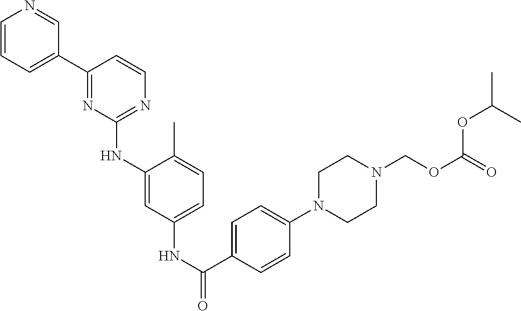 [SMILES: Cc1ccc(NC(=O)c2ccc(N3CCN(COC(=O)OC(C)C)CC3)cc2)cc1Nc1nccc(-c2cccnc2)n1] | Carbonic acid isopropyl ester 4-{4-[4-methyl-3-(4- pyridin-3-yl-pyrimidin-2-ylamino)-phenyl- carbamoyl]-phenyl}-piperazin-1-ylmethyl ester |
| 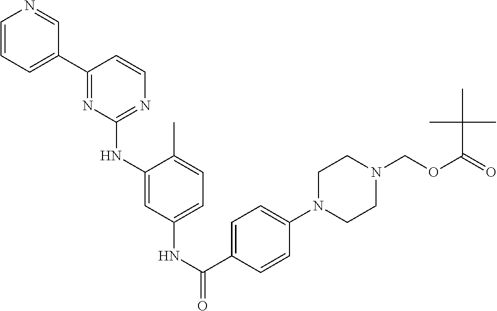 [SMILES: Cc1ccc(NC(=O)c2ccc(N3CCN(COC(=O)C(C)(C)C)CC3)cc2)cc1Nc1nccc(-c2cccnc2)n1] | 2,2-Dimethyl-propionic acid 4-{4-[4-methyl-3-(4- pyridin-3-yl-pyrimidin-2-ylamino)-phenyl- carbamoyl]-phenyl}-piperazin-1-ylmethyl ester |

In some embodiments, the compounds of the present invention are represented in Formula (V), or a pharmaceutically acceptable salt, solvate, hydrate, cocrystal, or prodrug thereof, wherein X₁, X₂, or X₃ is independently CH or N; R₈ or R₉ independently is hydrogen or the masking group defined above; R₄ is the same as defined in Formula (I). In an embodiment, R₄ is hydrogen or CF₃.

In an embodiment, R₈ or R₉ is independently selected from the group consisting of H,

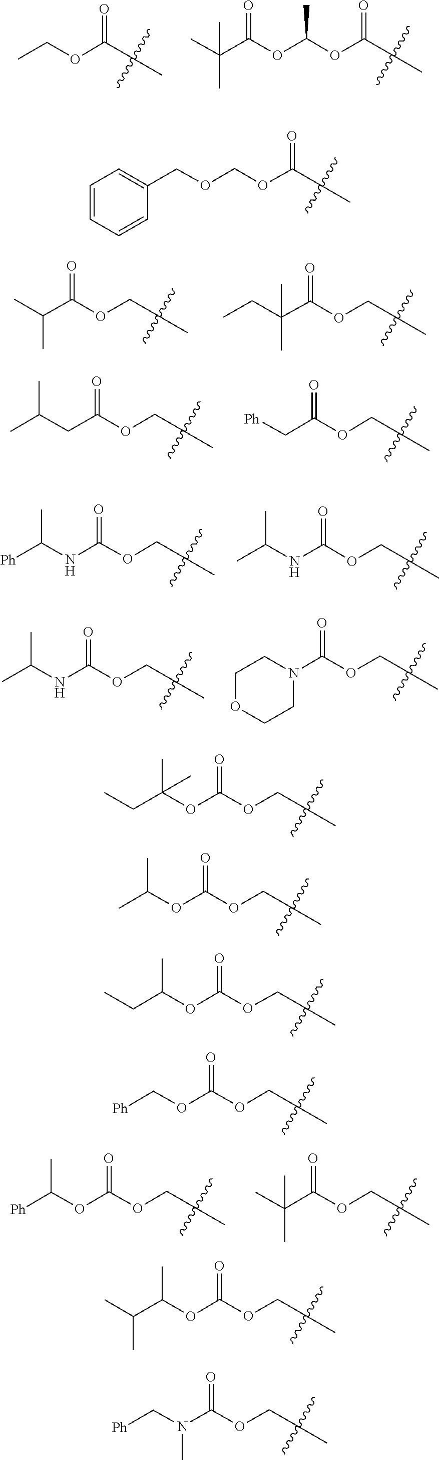

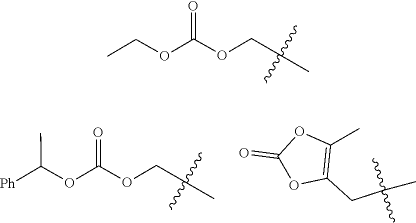

and combinations thereof.

Examples of compounds of Formula (IV) are listed in Table 4.

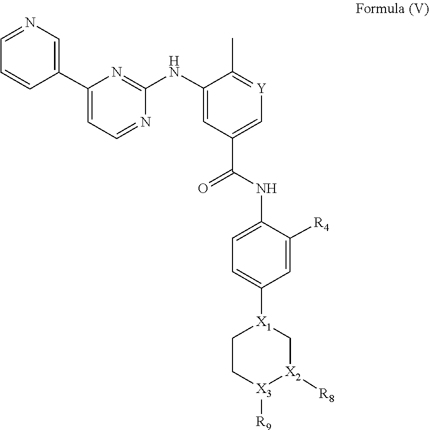

TABLE 4

| | |
|---|---|
| 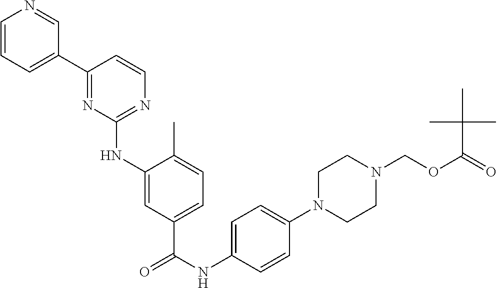 [SMILES: Cc1ccc(C(=O)Nc2ccc(N3CCN(COC(=O)C(C)(C)C)CC3)cc2)cc1Nc1nccc(-c2cccnc2)n1] | 2,2-Dimethyl-propionic acid 4-{4-[4-methyl-3-(4-pyridin-3-yl- pyrimidin-2-ylamino)-benzoylamino]-phenyl}-piperazin-1- ylmethyl ester |
| 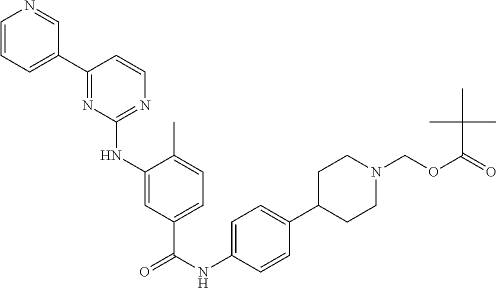 [SMILES: Cc1ccc(C(=O)Nc2ccc(C3CCN(COC(=O)C(C)(C)C)CC3)cc2)cc1Nc1nccc(-c2cccnc2)n1] | 2,2-Dimethyl-propionic acid 4-{4-[4-methyl-3-(4-pyridin-3-yl- pyrimidin-2-ylamino)-benzoylamino]phenyl}-piperidin-1- ylmethyl ester |
| 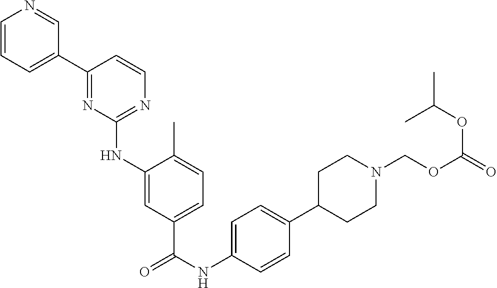 [SMILES: Cc1ccc(C(=O)Nc2ccc(C3CCN(COC(=O)OC(C)C)CC3)cc2)cc1Nc1nccc(-c2cccnc2)n1] | Carbonic acid isopropyl ester 4-{4-[4-methyl-3-(4-pyridin-3- yl-pyrimidin-2-ylamino)-benzoylamino]-phenyl}-piperidin-1- ylmethyl ester |
| 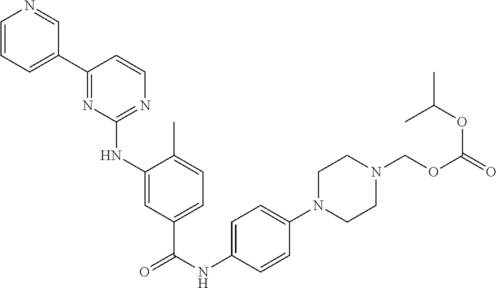 [SMILES: Cc1ccc(C(=O)Nc2ccc(N3CCN(COC(=O)OC(C)C)CC3)cc2)cc1Nc1nccc(-c2cccnc2)n1] | Carbonic acid isopropyl ester 4-{4-[4-methyl-3-(4-pyridin-3- yl-pyrimidin-2-ylamino)-benzoylamino]-phenyl}-piperazin-1- ylmethyl lester |
| 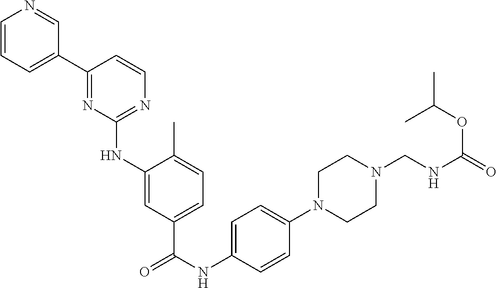 [SMILES: Cc1ccc(C(=O)Nc2ccc(N3CCN(CNC(=O)OC(C)C)CC3)cc2)cc1Nc1nccc(-c2cccnc2)n1] | (4-{4-[4-Methyl-3-(4-pyridin-3-yl-pyrimidin-2-ylamino)- benzoylamino]-phenyl}-piperazin-1-ylmethyl)-carbamic acid isopropyl ester |
| 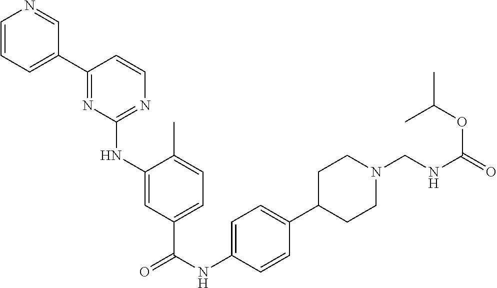 [SMILES: Cc1ccc(C(=O)Nc2ccc(C3CCN(CNC(=O)OC(C)C)CC3)cc2)cc1Nc1nccc(-c2cccnc2)n1] | (4-{4-[4-Methyl-3-(4-pyridin-3-yl-pyrimidin-2-ylamino)- benzoylamino]-phenyl}-piperidin-1-ylmethyl)-carbamic acid isopropyl ester |
| 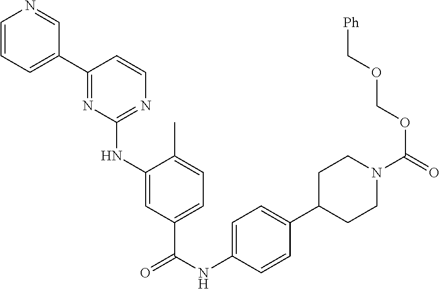 [SMILES: Cc1ccc(C(=O)Nc2ccc(C3CCN(C(=O)OCOCc4ccccc4)CC3)cc2)cc1Nc1nccc(-c2cccnc2)n1] | 4-{4-[4-Methyl-3-(4-pyridin-3-yl-pyrimidin-2-ylamino)- benzoylamino]-phenyl}-piperidine-1-carboxylic acid benzyloxymethyl ester |
| 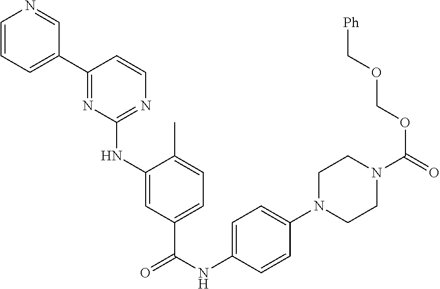 [SMILES: Cc1ccc(C(=O)Nc2ccc(N3CCN(C(=O)OCOCc4ccccc4)CC3)cc2)cc1Nc1nccc(-c2cccnc2)n1] | 4-{4-[4-Methyl-3-(4-pyridin-3-yl-pyrimidin-2-ylamino)- benzoylamino]-phenyl}-piperazine-1-carboxylic acid benzyloxymethyl ester |
| 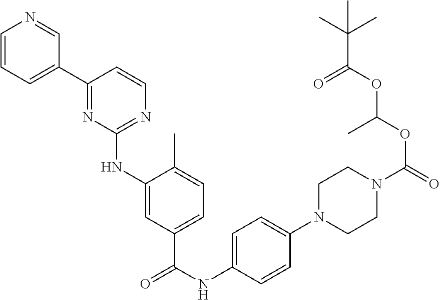 [SMILES: Cc1ccc(C(=O)Nc2ccc(N3CCN(C(=O)OC(C)OC(=O)C(C)(C)C)CC3)cc2)cc1Nc1nccc(-c2cccnc2)n1] | 4-{4-[4-Methyl-3-(4-pyridin-3-yl-pyrimidin-2-ylamino)- benzoylamino]-phenyl}-piperazine-1-carboxylic acid 1-(2,2- dimethyl-propionyloxy)-ethyl ester |
| 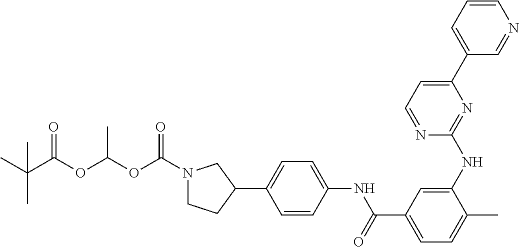 [SMILES: Cc1ccc(C(=O)Nc2ccc(C3CCN(C(=O)OC(C)OC(=O)C(C)(C)C)C3)cc2)cc1Nc1nccc(-c2cccnc2)n1] | 3-{4-[4-Methyl-3-(4-pyridin-3-yl-pyrimidin-2-ylamino)- benzoylamino]-phenyl}-pyrrolidine-1-carboxylic acid 1-(2,2- dimethyl-propionyloxy)-ethyl ester |
| 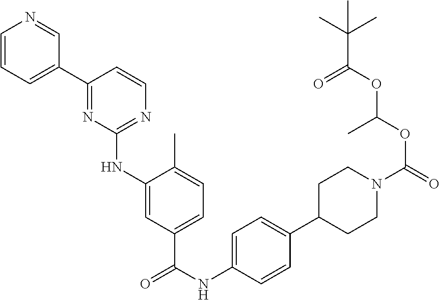 [SMILES: Cc1ccc(C(=O)Nc2ccc(C3CCN(C(=O)OC(C)OC(=O)C(C)(C)C)CC3)cc2)cc1Nc1nccc(-c2cccnc2)n1] | 4-{4-[4-Methyl-3-(4-pyridin-3-yl-pyrimidin-2-ylamino)- benzoylamino]-phenyl}-piperidine-1-carboxylic acid 1-(2,2- dimethyl-propionyloxy)-ethyl ester |
| 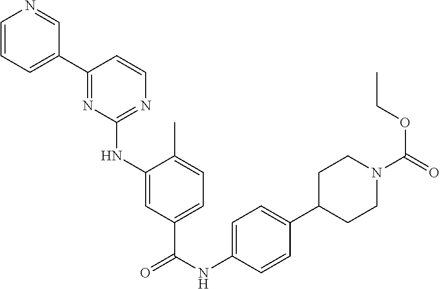 [SMILES: CCOC(=O)N1CCC(c2ccc(NC(=O)c3ccc(C)c(Nc4nccc(-c5cccnc5)n4)c3)cc2)CC1] | 4-{4-[4-Methyl-3-(4-pyridin-3-yl-pyrimidin-2-ylamino)- benzoylamino]-phenyl}-piperidine-1-carboxylic acid ethyl ester |
| 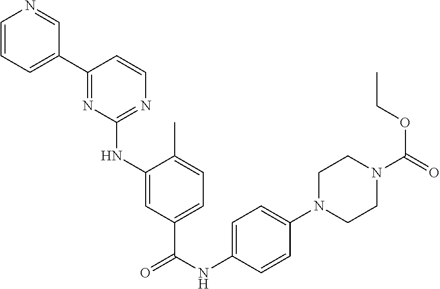 [SMILES: CCOC(=O)N1CCN(c2ccc(NC(=O)c3ccc(C)c(Nc4nccc(-c5cccnc5)n4)c3)cc2)CC1] | 4-{4-[4-Methyl-3-(4-pyridin-3-yl-pyrimidin-2-ylamino)- benzoylamino]-phenyl}-piperazine-1-carboxylic acid ethyl ester |
| 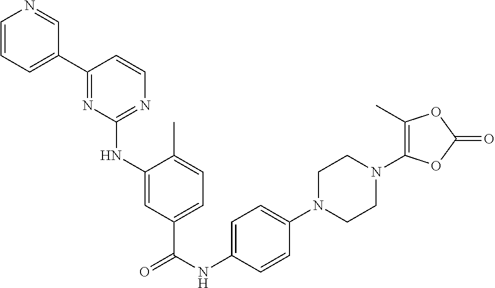 [SMILES: Cc1ccc(C(=O)Nc2ccc(N3CCN(c4oc(=O)oc4C)CC3)cc2)cc1Nc1nccc(-c2cccnc2)n1] | 4-Methyl-N-{4-[4-(5-methyl-2-oxo-[1,3]dioxol-4-yl)- piperazin-1-yl]-phenyl}-3-(4-pyridin-3-yl-pyrimidin-2- ylamino)-benzamide |
| 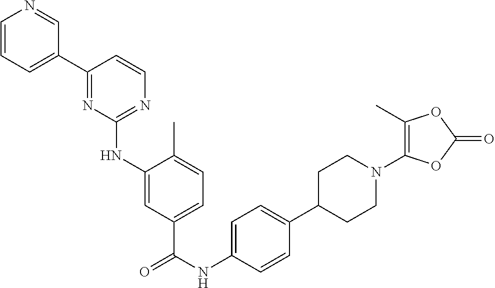 [SMILES: Cc1ccc(C(=O)Nc2ccc(C3CCN(c4oc(=O)oc4C)CC3)cc2)cc1Nc1nccc(-c2cccnc2)n1] | 4-Methyl-N-{4-[1-(5-methyl-2-oxo-[1,3]dioxol-4-yl)- piperidin-4-yl]-phenyl}-3-(4-pyridin-3-yl-pyrimidin-2- ylamino)-benzamide |
| 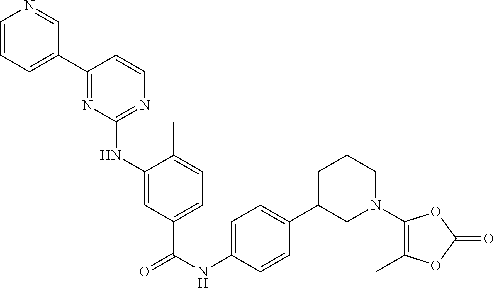 [SMILES: Cc1ccc(C(=O)Nc2ccc(C3CCCN(c4oc(=O)oc4C)C3)cc2)cc1Nc1nccc(-c2cccnc2)n1] | 4-Methyl-N-{4-[1-(5-methyl-2-oxo-[1,3]dioxol-4-yl)- piperidin-3-yl]-phenyl}-3-(4-pyridin-3-yl-pyrimidin-2- ylamino)-benzamide |
| 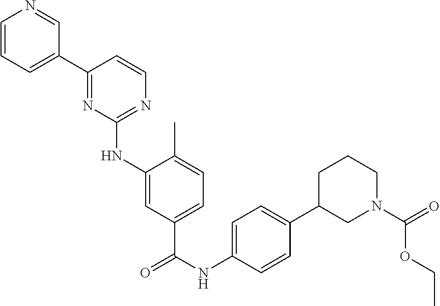 [SMILES: CCOC(=O)N1CCCC(c2ccc(NC(=O)c3ccc(C)c(Nc4nccc(-c5cccnc5)n4)c3)cc2)C1] | 3-{4-[4-Methyl-3-(4-pyridin-3-yl-pyrimidin-2-ylamino)- benzoylamino]-phenyl}-piperidine-1-carboxylic acid ethyl ester |
| 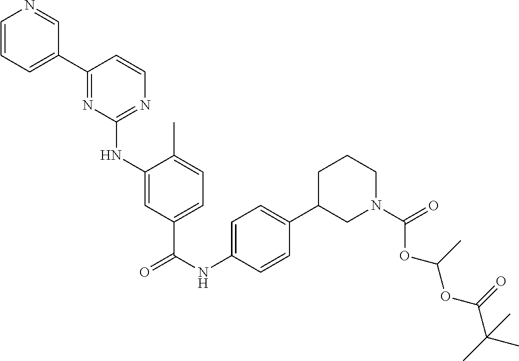 [SMILES: Cc1ccc(C(=O)Nc2ccc(C3CCCN(C(=O)OC(C)OC(=O)C(C)(C)C)C3)cc2)cc1Nc1nccc(-c2cccnc2)n1] | 3-{4-[4-Methyl-3-(4-pyridin-3-yl-pyrimidin-2-ylamino)- benzoylamino]-phenyl}-piperidine-1-carboxylic acid 1-(2,2- dimethyl-propionyloxy)-ethyl ester |
| 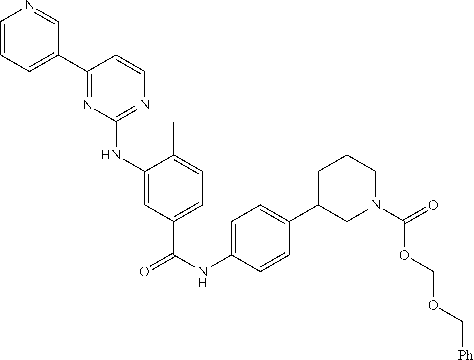 [SMILES: Cc1ccc(C(=O)Nc2ccc(C3CCCN(C(=O)OCOCc4ccccc4)C3)cc2)cc1Nc1nccc(-c2cccnc2)n1] | 3-{4-[4-Methyl-3-(4-pyridin-3-yl-pyrimidin-2-ylamino)- benzoylamino]-phenyl}-piperidine-1-carboxylic acid benzyloxymethyl ester |
| 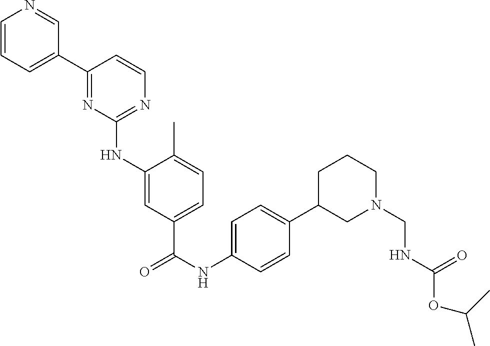 [SMILES: Cc1ccc(C(=O)Nc2ccc(C3CCCN(CNC(=O)OC(C)C)C3)cc2)cc1Nc1nccc(-c2cccnc2)n1] | (3-{4-[4-Methyl-3-(4-pyridin-3-yl-pyrimidin-2-ylamino)- benozylamino]-phenyl}-piperidin-1-ylmethyl)-carbamic acid isopropyl ester |
| 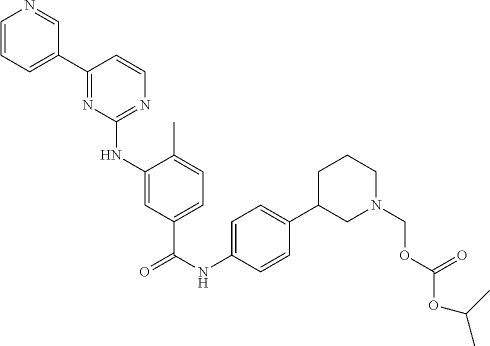 [SMILES: Cc1ccc(C(=O)Nc2ccc(C3CCCN(COC(=O)OC(C)C)C3)cc2)cc1Nc1nccc(-c2cccnc2)n1] | Carbonic acid isopropyl ester 3-{4-[4-methyl-3-(4-pyridin-3- yl-pyrimidin-2-yalmino)-benzoylamino]-phenyl}-piperidin-1- ylmethyl ester |
| 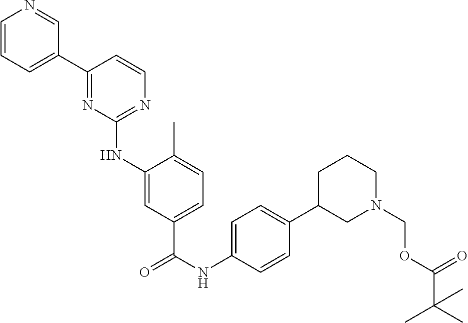 [SMILES: Cc1ccc(C(=O)Nc2ccc(C3CCCN(COC(=O)C(C)(C)C)C3)cc2)cc1Nc1nccc(-c2cccnc2)n1] | 2,2-Dimethyl-propionic acid 3-{4-[4-methyl-3-(4-pyridin-3-yl- pyrimidin-2-ylamino)-benzoylamino]-phenyl}-piperidin-1- ylmethyl ester |

The compounds of the present invention may contain asymmetric or chiral centers, and therefore, exist in different stereoisomeric forms. It is contemplated that all stereoisomeric forms of the compounds as well as mixtures thereof, including racemic mixtures, form part of the present invention. In addition, the present invention contemplates all geometric and positional isomers. For example, if the compound contains a double bond, both the cis and trans forms (designated as S and E, respectively), as well as mixtures, are contemplated.

Mixture of stereoisomers, such as diastereomeric mixtures, can be separated into their individual stereochemical components on the basis of their physical chemical differences by known methods such as chromatography and/or fractional crystallization. Enantiomers can also be separated by converting the enantiomeric mixture into a diastereomeric mixture by reaction with an appropriate optically active compound (e.g., an alcohol), separating the diastereomers and converting (e.g., hydrolyzing) the individual diastereomers to the corresponding pure enantiomers. Also, some compounds may be atropisomers (e.g., substituted biaryls).

The compounds of the present invention may exist in unsolvated as well as solvated forms with pharmaceutically acceptable solvents such as water (hydrate), ethanol, and the like. The present invention contemplates and encompasses both the solvated and unsolvated forms.

It is also possible that compounds of the present invention may exist in different tautomeric forms. All tautomers of compounds of the present invention are contemplated. For example, all of the tautomeric forms of the tetrazole moiety are included in this invention. Also, for example, all keto-enol or imine-enamine forms of the compounds are included in this invention.

Those skilled in the art will recognize that the compound names and structures contained herein may be based on a particular tautomer of a compound. While the name or structure for only a particular tautomer may be used, it is intended that all tautomers are encompassed by the present invention, unless stated otherwise.

It is also intended that the present invention encompass compounds that are synthesized in vitro using laboratory techniques, such as those well known to synthetic chemists; or synthesized using in vivo techniques, such as through metabolism, fermentation, digestion, and the like. It is also contemplated that the compounds of the present invention may be synthesized using a combination of in vitro and in vivo techniques.

The present invention also includes isotopically-labelled compounds, which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁶O, ¹⁷O, ¹⁸O, ³¹P, ³²P, ³⁵S, ¹⁸F, and ³⁶Cl. In one aspect, the present invention relates to compounds wherein one or more hydrogen atom is replaced with deuterium (²H) atoms.

### Methods of the Invention

The compounds of the invention, and pharmaceutically acceptable salts thereof, are useful for treating a subject suffering from Parkinson's Disease, Alzheimer's Disease, amyotrophic lateral sclerosis, or Huntington's disease. For example, the compounds are useful for treating Alzheimer's disease, for helping prevent or delay the onset of Alzheimer's disease, for treating patients with MCI (mild cognitive impairment) and preventing or delaying the onset of Alzheimer's disease in those who would progress from MCI to AD, for treating cerebral amyloid angiopathy and preventing its potential consequences, i.e. single and recurrent lobal hemorrhages, for treating other degenerative dementias, including dementias of mixed vascular and degenerative origin, dementia associated with Parkinson's disease, dementia associated with progressive supranuclear palsy, dementia associated with cortical basal degeneration, and diffuse Lewy body type Alzheimer's disease. The compounds and compositions of the invention are particularly useful for treating or preventing Alzheimer's disease. When treating or preventing these diseases, the compounds of the invention can either be used individually or in combination, as is best for the patient.

As used herein, the term “treating” means that the compounds of the invention can be used in humans with at least a tentative diagnosis of disease. The compounds of the invention will delay or slow the progression of the disease thereby giving the individual a more useful life span.

The term “preventing” means that the compounds of the present invention are useful when administered to a patient who has not been diagnosed as possibly having the disease at the time of administration, but who would normally be expected to develop the disease or be at increased risk for the disease. The compounds of the invention will slow the development of disease symptoms, delay the onset of the disease, or prevent the individual from developing the disease at all. Preventing also includes administration of the compounds of the invention to those individuals thought to be predisposed to the disease due to age, familial history, genetic or chromosomal abnormalities, and/or due to the presence of one or more biological markers for the disease, such as a known genetic mutation of APP or APP cleavage products in brain tissues or fluids.

In treating or preventing the above diseases, the compounds of the invention are administered in a therapeutically effective amount. The therapeutically effective amount will vary depending on the particular compound used and the route of administration, as is known to those skilled in the art.

In treating a patient displaying any of the diagnosed above conditions a physician may administer a compound of the invention immediately and continue administration indefinitely, as needed. In treating patients who are not diagnosed as having Alzheimer's disease, but who are believed to be at substantial risk for Alzheimer's disease, the physician should preferably start treatment when the patient first experiences early pre-Alzheimer's symptoms such as, memory or cognitive problems associated with aging. In addition, there are some patients who may be determined to be at risk for developing Alzheimer's through the detection of a genetic marker such as APOE4 or other biological indicators that are predictive for Alzheimer's disease. In these situations, even though the patient does not have symptoms of the disease, administration of the compounds of the invention may be started before symptoms appear, and treatment may be continued indefinitely to prevent or delay the outset of the disease.

In another aspect, the present disclosure provides a method of treating a cancer, comprising administering to a subject in need thereof a pharmaceutically effective amount of the compound described herein or a pharmaceutically acceptable salt, solvate, hydrate, cocrystal, or prodrug thereof.

In another aspect, the present disclosure provides a method of treating a cancer, comprising administering to a subject in need thereof a pharmaceutically effective amount of a composition comprising the compound described herein or a pharmaceutically acceptable salt, solvate, hydrate, cocrystal, or prodrug thereof and a pharmaceutically acceptable excipient.

In one embodiment, the subject is a human.

In one embodiment, the cancer is selected from the group consisting of bladder cancer, head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, kidney cancer, liver cancer, esophageal cancer, testicular cancer, gynecological cancer, thyroid cancer, Kaposi's sarcoma, viral-induced cancer, glioblastoma, glioblastoma multiforme, non-small-cell lung cancer, hepatocellular carcinoma, metastatic colon cancer, multiple myeloma, small-cell lung cancer, melanoma, and combinations thereof.

In one embodiment, the cancer is a hematologic malignancy selected from the group consisting of lymphoma, leukemia, multiple myeloma, acute lymphatic leukemia (ALL), chronic myeloid leukemia (CML), acute myeloid leukemia (AML), mantle cell lymphoma, and T cell lymphoma, hematological neoplasms, diffuse large B-cell lymphoma, follicle center lymphoma, Hodgkin's disease, non-Hodgkin's lymphoma, chronic lymphocytic leukemia (CLL), B-cell non-Hodgkin lymphoma, primary central nervous system lymphoma, myelodysplasia syndrome (MDS), and myeloproliferative diseases.

### Dosage Forms and Amounts

The compounds of the invention can be administered orally, parenternally, (IV, IM, depo-IM, SQ, and depo SQ), sublingually, intranasally (inhalation), intrathecally, topically, or rectally. Dosage forms known to those of skill in the art are suitable for delivery of the compounds of the invention.

Compositions are provided that contain therapeutically effective amounts of the compounds of the invention. The compounds are preferably formulated into suitable pharmaceutical preparations such as tablets, capsules, or elixirs for oral administration or in sterile solutions or suspensions for parenternal administration. Typically the compounds described above are formulated into pharmaceutical compositions using techniques and procedures well known in the art.

About 1 to 500 mg of a compound or mixture of compounds of the invention or a physiologically acceptable salt or ester is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare compositions, one or more compounds of the invention are mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods are known and include, but are not limited to, using cosolvents such as dimethylsulfoxide (DMSO), using surfactants such as Tween (D, and dissolution in aqueous sodium bicarbonate. Derivatives of the compounds, such as salts or prodrugs may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

The compounds of the invention may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder.

The compounds and compositions of the invention can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampules, vials, and the like for parenternal administration; and patches, medipads, creams, and the like for topical administration.

The concentration of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action.

Solutions or suspensions used for parenternal, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenternal preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known for example, as described in U.S. Pat. No. 4,522,811.

The active compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

Compounds of the invention may be administered enterally or parenterally. When administered orally, compounds of the invention can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, it is preferred that they be of the sustained release type so that the compounds of the invention need to be administered only once or twice daily.

The oral dosage forms are administered to the patient 1, 2, 3, or 4 times daily. It is preferred that the compounds of the invention be administered either three or fewer times, more preferably once or twice daily. Hence, it is preferred that the compounds of the invention be administered in oral dosage form. It is preferred that whatever oral dosage form is used, that it be designed so as to protect the compounds of the invention from the acidic environment of the stomach. Enteric coated tablets are well known to those skilled in the art. In addition, capsules filled with small spheres each coated to protect from the acidic stomach, are also well known to those skilled in the art.

When administered orally, an administered amount therapeutically effective to treat or prevent AD is from about 0.1 mg/day to about 1,000 mg/day. It is preferred that the oral dosage is from about 1 mg/day to about 100 mg/day. It is more preferred that the oral dosage is from about 5 mg/day to about 50 mg/day. It is understood that while a patient may be started at one dose, that dose may be varied over time as the patient's condition changes.

Compounds of the invention may also be advantageously delivered in a nano crystal dispersion formulation. Preparation of such formulations is described, for example, in U.S. Pat. No. 5,145,684. Nano crystalline dispersions of HIV protease inhibitors and their method of use are described in U.S. Pat. No. 6,045,829. The nano crystalline formulations typically afford greater bioavailability of drug compounds.

The compounds of the invention can be administered parenterally, for example, by IV, IM, depo-IM, SC, or depo-SC. When administered parenterally, a therapeutically effective amount of about 0.5 to about 100 mg/day, preferably from about 5 to about 50 mg daily should be delivered. When a depot formulation is used for injection once a month or once every two weeks, the dose should be about 0.5 mg/day to about 50 mg/day, or a monthly dose of from about 15 mg to about 1,500 mg. In part because of the forgetfulness of the patients with Alzheimer's disease, it is preferred that the parenteral dosage form be a depo formulation.

The compounds of the invention can be administered sublingually. When given sublingually, the compounds of the invention should be given one to four times daily in the amounts described above for IM administration.

The compounds of the invention can be administered intranasally. When given by this route, the appropriate dosage forms are a nasal spray or dry powder, as is known to those skilled in the art. The dosage of the compounds of the invention for intranasal administration is the amount described above for IM administration.

The compounds of the invention can be administered intrathecally. When given by this route the appropriate dosage form can be a parenternal dosage form as is known to those skilled in the art. The dosage of the compounds of the invention for intrathecal administration is the amount described above for IM administration.

The compounds of the invention can be administered topically. When given by this route, the appropriate dosage form is a cream, ointment, or patch. Because of the amount of the compounds of the invention to be administered, the patch is preferred. When administered topically, the dosage is from about 0.5 mg/day to about 200 mg/day. Because the amount that can be delivered by a patch is limited, two or more patches may be used. The number and size of the patch is not important, what is important is that a therapeutically effective amount of the compounds of the invention be delivered as is known to those skilled in the art. The compounds of the invention can be administered rectally by suppository as is known to those skilled in the art. When administered by suppository, the therapeutically effective amount is from about 0.5 mg to about 500 mg.

The compounds of the invention can be administered by implants as is known to those skilled in the art. When administering a compound of the invention by implant, the therapeutically effective amount is the amount described above for depot administration.

The compounds of the invention are used in the same manner, by the same routes of administration, using the same pharmaceutical dosage forms, and at the same dosing schedule as described above, for preventing disease or treating patients with MCI (mild cognitive impairment) and preventing or delaying the onset of Alzheimer's disease in those who would progress from MCI to AD, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, and diffuse Lewy body type of Alzheimer's disease.

The compounds of the invention can be used in combination, with each other or with other therapeutic agents or approaches used to treat or prevent the conditions listed above. Such agents or approaches include: acetylcholine esterase inhibitors such as tacrine (tetrahydroaminoacridine, marketed as COGNEX®), donepezil hydrochloride, (marketed as Aricept® and rivastigmine (marketed as Exelon®); gamma-secretase inhibitors; anti-inflammatory agents such as cyclooxygenase II inhibitors; anti-oxidants such as Vitamin E and ginkolides; immunological approaches, such as, for example, immunization with A beta peptide or administration of anti-A beta peptide antibodies; statins; and direct or indirect neurotropic agents such as Cerebrolysin®, AIT-082 (Emilieu, 2000, Arch. Neurol. 57:454), and other neurotropic agents of the future.

It should be apparent to one skilled in the art that the exact dosage and frequency of administration will depend on the particular compounds of the invention administered, the particular condition being treated, the severity of the condition being treated, the age, weight, general physical condition of the particular patient, and other medication the individual may be taking as is well known to administering physicians who are skilled in this art.

### EXAMPLES

### Synthetic Methods

Compound in accordance with Formula (I) can be made through the application of the following methodologies.

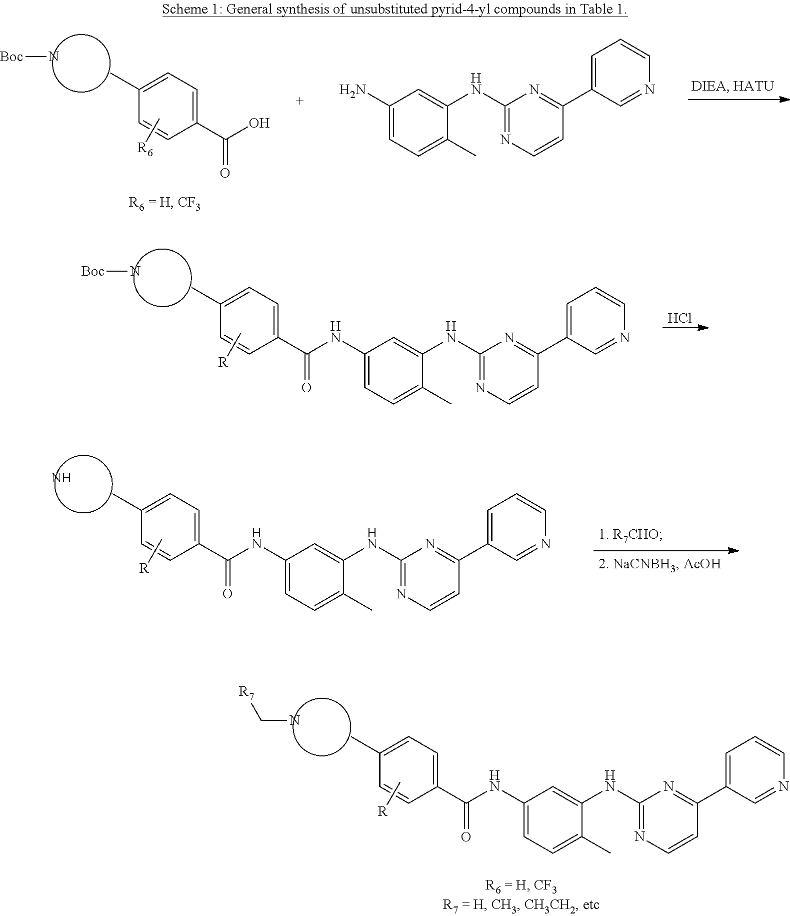

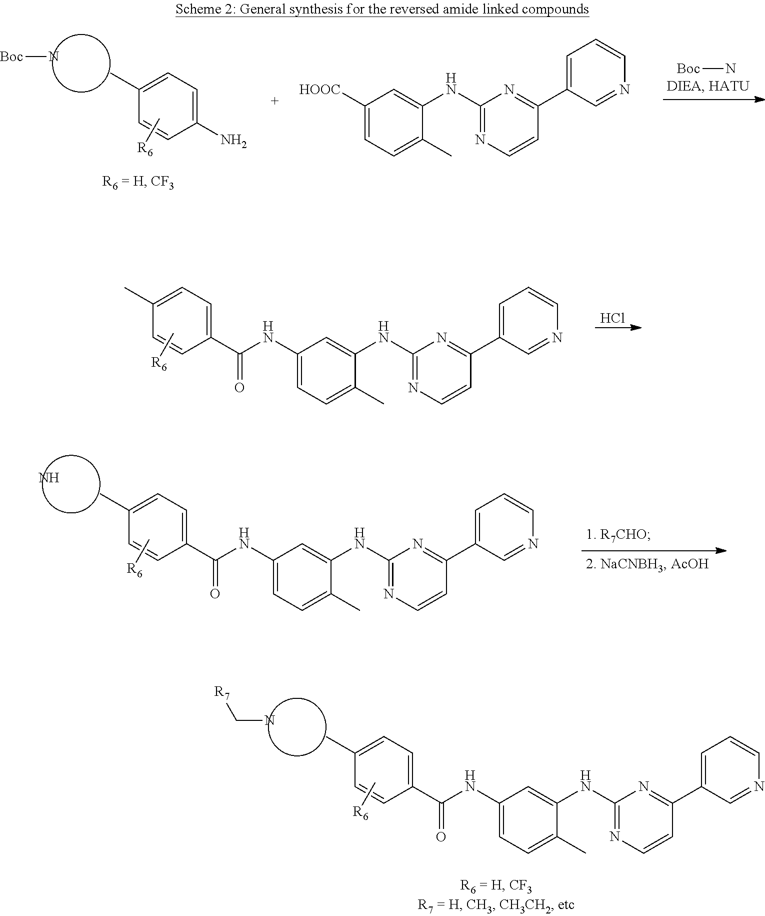

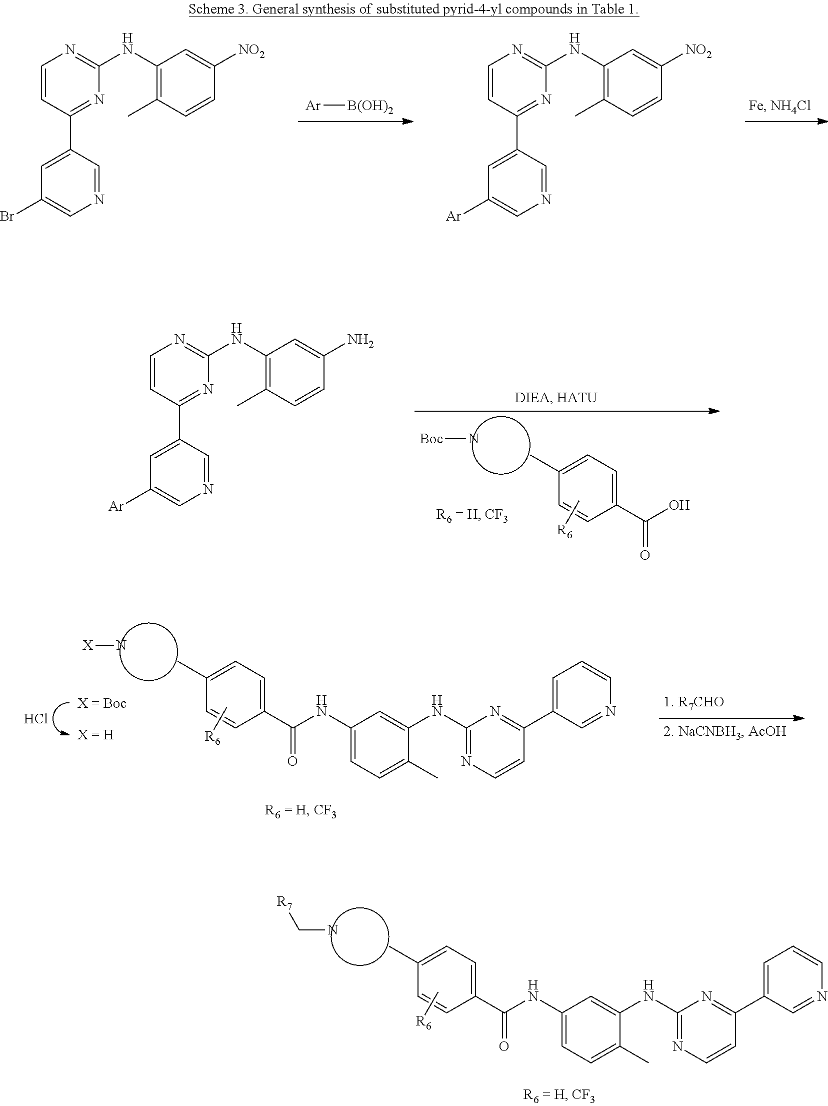

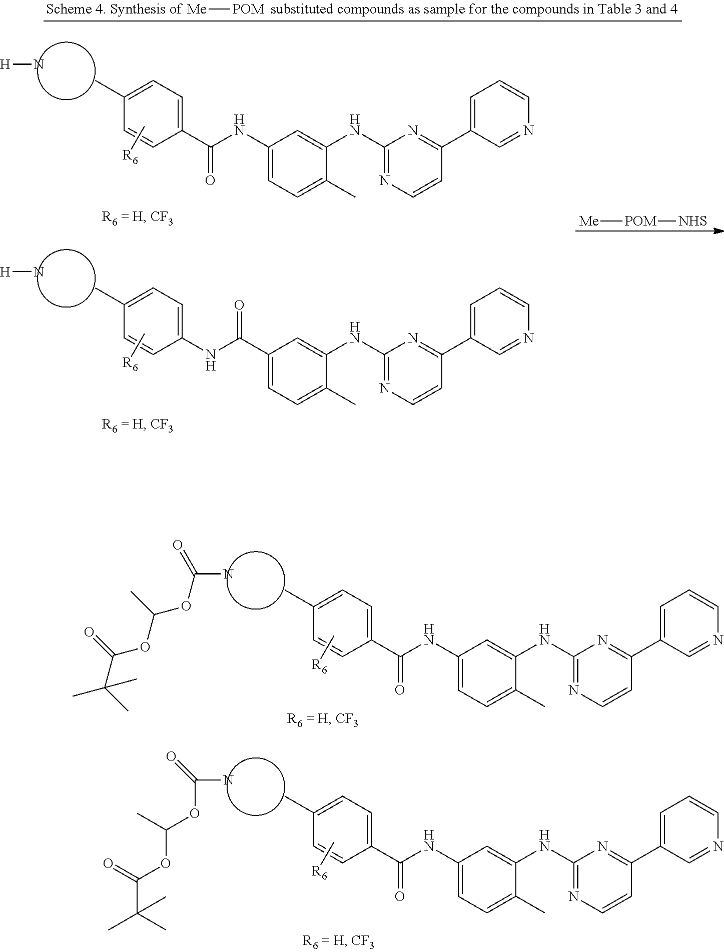

### Example 1: N-(4-(2-(dimethylamino)propyl)phenyl)-4-methyl-3-((2-(pyridin-3-yl)pyrimidin-4-yl)amino)benzamide

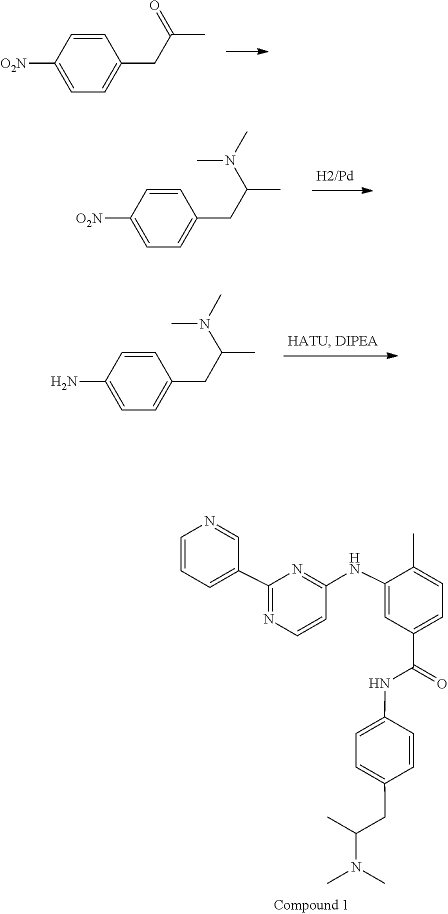 [SMILES: CC(=O)Cc1ccc([N+](=O)[O-])cc1 ; CC(Cc1ccc([N+](=O)[O-])cc1)N(C)C ; CC(Cc1ccc(N)cc1)N(C)C ; Cc1ccc(C(=O)Nc2ccc(CC(C)N(C)C)cc2)cc1Nc1ccnc(-c2cccnc2)n1]

### Step 1: Dimethyl-[1-methyl-2-(4-nitro-phenyl)-ethyl]-amine

To a solution of 1-(4-Nitro-phenyl)-propan-2-one (0.7 g, 4 mmol) in 100 mL of CH₂Cl₂ was added Dimethylamine in MeOH (1M, 6 mL) under nitrogen protection at room temperature, stirred for 30 min, then NaBH(OAc)₃ (1.5 g) portion wisely in 3 hours. The reaction mixture was stirred at room temperature for overnight and quenched with water. The separated aqueous layer was neutralized with 4N of aq. NaOH to pH=8-9 and extracted with CH₂Cl₂. The organic layer was dried by Na₂SO₄, concentered. The residue was slurried in MTBE and 4N HCl in dioxane (5 mL) was added. Precipitates were collected by filtration to give Dimethyl-[1-methyl-2-(4-nitro-phenyl)-ethyl]-amine (HCl slat) as off-white solids (0.8 g, 81%).

### Step 2: 4-(2-Dimethylamino-propyl)-phenylamine

To a solution of Dimethyl-[1-methyl-2-(4-nitro-phenyl)-ethyl]-amine (0.8 g) in 100 mL of MeOH was added Pd (c) 10% (100 mg) under nitrogen protection. The reaction mixture was purged with H2 three times and stirred under H2 atmosphere for overnight. The reaction mixture was filtrated through a celite pad and filtrate was concentered to give 4-(2-Dimethylamino-propyl)-phenylamine (0.7 g).

### Step 3: N-(4-(2-(dimethylamino)propyl)phenyl)-4-methyl-3-((2-(pyridin-3-yl)pyrimidin-4-yl)amino)benzamide

To a suspension of 3-(2-Pyridin-3-yl-pyrimidin-4-ylamino)-benzoic acid (0.7 g) in 100 mL of DMF at 0° C. was added 4-(2-Dimethylamino-propyl)-phenylamine (0.5 g), HATU (0.8 g), and DIPEA (1.2 mL). The reaction mixture was stirred at room temperature overnight before pure into ice water (800 mL). The mixture extracted with EtOAc, washed with brine, dried over Na2SO4, and concentrated. The collected sticky fine solid was slurried in MTBE and stirred at room temperature for 2 hours, filtered and dried to obtain Compound 1 as yellow solid (0.9 g, 74%). MS: [M+H]⁺: 467.2; HPLC purity: 100.0%

### Example 2: 4-methyl-N-(4-(1-methylpiperidin-4-yl)phenyl)-3-((2-(pyridin-3-yl)pyrimidin-4-yl)amino)benzamide

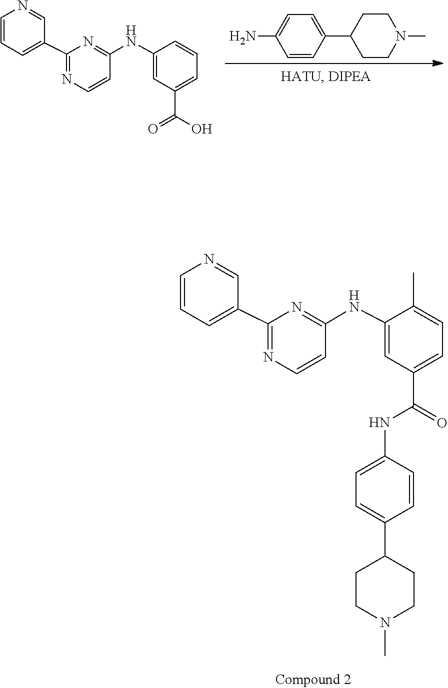 [SMILES: O=C(O)c1cccc(Nc2ccnc(-c3cccnc3)n2)c1 ; CN1CCC(c2ccc(N)cc2)CC1 ; Cc1ccc(C(=O)Nc2ccc(C3CCN(C)CC3)cc2)cc1Nc1ccnc(-c2cccnc2)n1]

To a suspension of 3-(2-Pyridin-3-yl-pyrimidin-4-ylamino)-benzoic acid (0.7 g) in 100 mL of DMF at 0° C. was added 4-(1-Methyl-piperidin-4-yl)-phenylamine (0.6 g), HATU (0.9 g), and DIPEA (1.3 mL). The reaction mixture was stirred at room temperature overnight before pure into ice water. The slurry was stirred at room temperature, filtrated, and washed with MTBE to get Compound 2 as pale brown solids (1.1 g, 81%). MS: [M+H]⁺: 479.2; HPLC purity: 97.9%.

### Example 3: Assay for Levels of Aβ40 and A1342

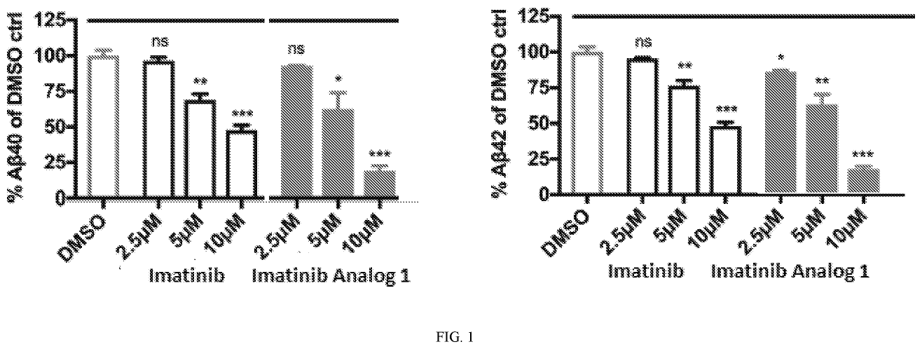

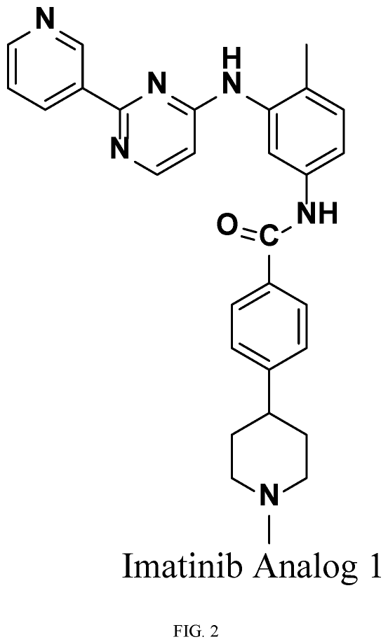

Aβ40 and Aβ42 peptides are the main building blocks for the formation of toxic AP. Inhibiting production of Aβ40 and Aβ42 and/or increasing clearance of Aβ40 and Aβ42 are the important strategies of drug development for the treatment of AD. The developmental γ-secretase inhibitors, BACE inhibitors and AP antibodies belong to this category. Anti-cancer drugs Imatinib and Nilotinib are found to be able to decrease AP and tau aggregate, two pathogenic hallmarks of AD. Imatinib and Nilotinib lower the levels of Aβ40 and Aβ42 by inhibiting production of Aβ40 and Aβ42 as well as mediate autophagy degradation pathways. Due to their limited brain permeability and mild potency, more potent Imatinib and Nilotinib analogs with improved brain penetration were developed and disclosed herein. For example, in the ELISA assay, Imatinib analog 1 is more potent than Imatinib on reducing Aβ40 and Aβ42 levels (FIGS. 1 and 2). These Imatinib analogs also showed in vivo efficacy in AD animal studies (Sun. J. Med. Chem. 2019, 3122-3134)

In the cell based assays, 6-well tissue culture plates (Corning) were seeded at 4.0×10⁵-4.5×10⁵N2a695 cells/mL, 2 mL/well for overnight incubation. Media were carefully removed and fresh media containing certain concentration of compounds were gently layered onto adherent cells (>95% confluent). After cells were incubated with compounds for 5 h at 37° C. in 5% CO₂, culture media were collected. To measure soluble AP concentrations in culture media, these were transferred to strips of 96-well plate for human Aβ40 peptide or to 96-well V-Plex Plus MSD (Mesoscale Discovery) plate for Ab Peptide Panel 1 (6E10) Kit (Catalog number K15200G) and processed as per manufacturer instructions. Signals for Ab were measured using Perkin Elmer Envision and SQ120 MSD ELISA reader.

The compound 2 is a Nilotinib analog. The compounds 1 and 2 and other Nilotinib analogs described herein showed much improved brain permeability compared to Nilotinib and Imatinib as demonstrated in in vivo PK studies.

### Example 4: In Vivo PK Study

In the PK profiling study, Compounds 1 and 2 were administered orally to three Sprague Dawley rats. The animals were fasted overnight before drug administration. The food supply were resumed 4 hrs post dose. Blood samples were collected at 8 time points after dose: 0.25 h, 0.5 h, 1 h, 2 h, 4 h, 6 h, 8 h and 24 h. There are three samples for each time point. Brain tissues were collected after 24 hour blood sample collection. Drug concentrations in blood and brain were analyzed by LC-MS/MS. The analytical results were confirmed using quality control samples for intra-assay variation. The accuracy of >66.7% of the quality control samples should be between 80-120% of the known values. Standard set of parameters including area under the curve (AUC₍₀₋ₜ₎ and AUC_{(0-∞)}), elimination half-life (T_{1/2}), maximum plasma concentration (Cₘₐₓ), time to reach maximum plasma concentration (Tₘₐₓ) were calculated using noncompartmental analysis modules in FDA certified pharmacokinetic program Phoenix WinNonlin 7.0 (Pharsight, USA). The ratio of brain to plasma concentration were calculated by the concentration in brain tissue versus the concentration in plasma. In the animal studies and clinical trial, the ratio of CSF to plasma of Nilotinib in patients is only about 0.5-1%. With higher potency and significantly improved brain permeability, these Nilotinib analogs have great potential for the treatment of neurodegenerative diseases including AD, PD and other diseases.

TABLE 5
PK Parameters of Compounds 1 and 2

| | | | | Cmax | AUC₍₀₋ₜ₎ | AUC_{(0-∞)} | AUC_{(0-t) Brain}/ |
|---|---|---|---|---|---|---|---|
| Compound | Tissue | T1/2 (h) | Tmax (h) | ng/mL | h*ng/mL | h*ng/mL | AUC_{(0-t) plasma} |
| 1 | Plasma | 3.98 | 4.00 | 6675.33 | 64018.72 | 65367.69 | 12.1% |
| | Brain | 7.10 | 6.00 | 557.24 | 7715.31 | 8742.29 | |
| 2 | Plasma | 26.84 | 6.00 | 5375.86 | 91970.52 | 218573.64 | 14.2% |
| | Brain | 20.23 | 6.00 | 796.38 | 13094.62 | 25641.86 | |

1. A compound of Formula (I) or a pharmaceutically acceptable salt, solvate, hydrate, cocrystal, or prodrug thereof, 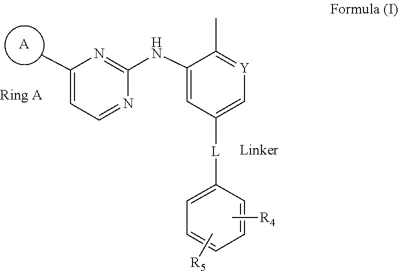
wherein Ring A is a substituted or unsubstituted 6-membered heteroaryl ring containing 1 or 2 nitrogen atoms with the remaining ring atoms being carbon;
L is a linker is selected from the group consisting of —NHC(O)—, —C(O)NH—, —SO₂NH—, —NHSO₂—, —C(CF₃)NH—, —NH—C(CF₃)—, —C(CH₂CH₂)—NH—, —NH(CH₂CH₂)C—, —C(F)═CH—, —CH═(F)C—, —C(CH₂OCH₂)NH—, —NH(CH₂OCH₂)C—, —C(CH₂OCH₂)O—, —O(CH₂OCH₂)C—, and combinations thereof;
Y═CH, or N;
R₄ is selected from the group consisting of hydrogen, halogens, —OMe, —CF₃, cyano, and ethylene; and
R₅ is an unsubstituted or substituted alkylamine.

2. The compound of claim 1, wherein Ring A is selected from: 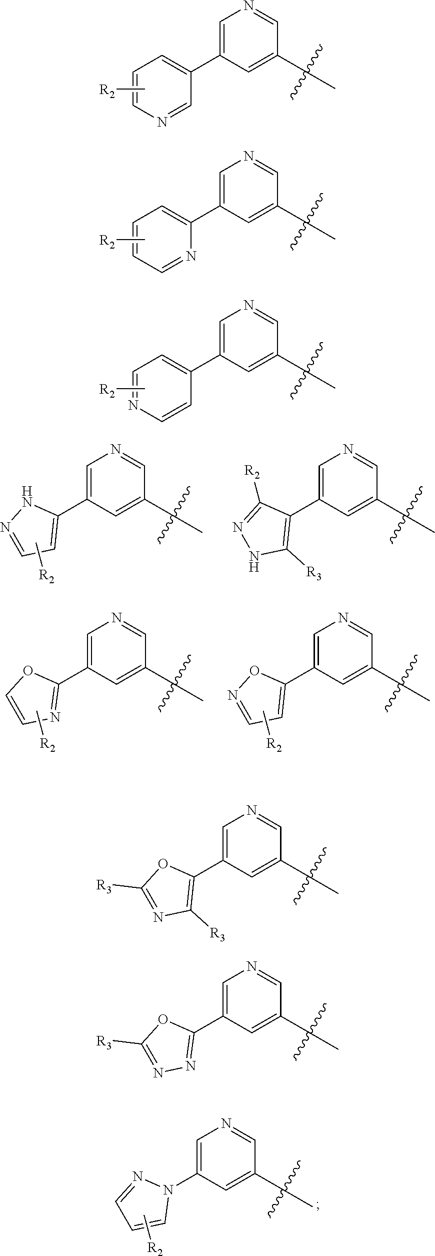
wherein R₂ and R₃ independently for each occurrence are selected from the group consisting of hydrogen, halogen, (C1-C6)alkyl, amino, cyano, (C2-C8)alkynyl, mono(C1-C6)alkylamino, di(C1-C6)alkylamino, and (C1-C6)alkyoxy.

3. The compound of claim 1, wherein R₅ is selected from the group consisting of dimethylaminoethyl, diethylaminoethyl, dipropylaminoethyl, 2-(dimethylamino)propyl, 3-piperidinyl, 1-methyl-pyrrolidin-3-yl, 1-ethyl-pyrrolidin-3-yl, 1-propyl-pyrrolidin-3-yl, 1-isopropyl-pyrrolidin-3-yl, 1-iso-butyl-pyrrolidin-3-yl, t-butyl-pyrrolidin-3-yl, 1-neo-pentyl-pyrrolidin-3-yl, 1-methyl-piperidin-3-yl, 1-ethyl-piperidin-3-yl, 1-propyl-piperidin-3-yl, 1-butyl-piperidin-3-yl, 1-isopropyl-piperidin-3-yl, 1-iso-butyl-piperidin-3-yl, 1-t-butyl-piperidin-3-yl, 1-neo-pentyl-piperidin-3-yl, 1-methyl-piperidin-4-yl, 1-ethyl-piperidin-4-yl, 1-propyl-piperidin-4-yl, 1-isopropyl-piperidin-4-yl, 1-iso-butyl-piperidin-4-yl, 1-t-butyl)-piperidin-4-yl, 1-neo-pentyl-piperidin-4-yl, 4-methyl-piperazin-1-yl, 4-ethyl-piperazin-1-yl, 4-propyl-piperazin-1-yl, 4-isopropyl-piperazin-1-yl, 4-iso-butyl-piperazin-1-yl, 4-t-butyl-piperazin-1-yl, 4-neo-pentyl-piperazin-1-yl, and 1-methyl-azepan-3-yl, 1-ethyl-azepan-3-yl, 1-propyl-azepan-3-yl, 1-ethyl-azepan-3-yl, 4-(4-Methyl-piperazin-1-yl)-2-trifluoromethyl-phenyl, 4-(4-Ethyl-piperazin-1-yl)-2-trifluoromethyl-phenyl, 4-(4-Propyl-piperazin-1-yl)-2-trifluoromethyl-phenyl, 4-(4-Isopropyl-piperazin-1-yl)-2-trifluoromethyl-phenyl, 4-(4-Isobutyl-piperazin-1-yl)-2-trifluoromethyl-phenyl, 4-(4-Isobutyl-piperazin-1-yl)-2-trifluoromethyl-phenyl, 4-(4-t-Butyl-piperazin-1-yl)-2-trifluoromethyl-phenyl, 4-(4-neo-Pentyl-piperazin-1-yl)-2-trifluoromethyl-phenyl, 4-(1-Methyl-piperidin-4-yl)-2-trifluoromethyl-phenyl, 4-(1-Ethyl-piperidin-4-yl)-2-trifluoromethyl-phenyl, 4-(1-Propyl-piperidin-4-yl)-2-trifluoromethyl-phenyl, 4-(1-Isopropyl-piperidin-4-yl)-2-trifluoromethyl-phenyl, 4-(1-Isobutyl-piperidin-4-yl)-2-trifluoromethyl-phenyl, 4-(1-t-Butyl-piperidin-4-yl)-2-trifluoromethyl-phenyl, 4-(1-neo-pentyl-piperidin-4-yl)-2-trifluoromethyl-phenyl, 4-(1-Methyl-piperidin-3-yl)-2-trifluoromethyl-phenyl, 4-(1-Ethyl-piperidin-3-yl)-2-trifluoromethyl-phenyl, 4-(1-Propyl-piperidin-3-yl)-2-trifluoromethyl-phenyl, 4-(1-Isopropyl-piperidin-3-yl)-2-trifluoromethyl-phenyl, 4-(1-Isobutyl-piperidin-3-yl)-2-trifluoromethyl-phenyl, 4-(1-t-Butyl-piperidin-3-yl)-2-trifluoromethyl-phenyl, 4-(1-neo-pentyl-piperidin-3-yl)-2-trifluoromethyl-phenyl, 3-(4-Methyl-piperazin-1-yl)-5-trifluoromethyl-phenyl, 3-(4-Ethyl-piperazin-1-yl)-5-trifluoromethyl-phenyl, 3-(4-Propyl-piperazin-1-yl)-5-trifluoromethyl-phenyl, 3-(4-Isopropyl-piperazin-1-yl)-5-trifluoromethyl-phenyl, 3-(4-Isobutyl-piperazin-1-yl)-5-trifluoromethyl-phenyl, 3-(4-t-Butyl-piperazin-1-yl)-5-trifluoromethyl-phenyl, 3-(4-neo-pentyl-piperazin-1-yl)-5-trifluoromethyl-phenyl, 3-(1-Methyl-piperidin-4-yl)-5-trifluoromethyl-phenyl, 3-(1-Ethyl-piperidin-4-yl)-5-trifluoromethyl-phenyl, 3-(1-Propyl-piperidin-4-yl)-5-trifluoromethyl-phenyl, 3-(1-Isopropyl-piperidin-4-yl)-5-trifluoromethyl-phenyl, 3-(1-Isobutyl-piperidin-4-yl)-5-trifluoromethyl-phenyl, 3-(1-t-Butyl-piperidin-4-yl)-5-trifluoromethyl-phenyl, 3-(1-neo-pentyl-piperidin-4-yl)-5-trifluoromethyl-phenyl, 3-(1-Methyl-piperidin-3-yl)-5-trifluoromethyl-phenyl, 3-(1-Ethyl-piperidin-3-yl)-5-trifluoromethyl-phenyl, 3-(1-Propyl-piperidin-3-yl)-5-trifluoromethyl-phenyl, 3-(1-Isopropyl-piperidin-3-yl)-5-trifluoromethyl-phenyl, 3-(1-Isobutyl-piperidin-3-yl)-5-trifluoromethyl-phenyl, 3-(1-t-Butyl-piperidin-3-yl)-5-trifluoromethyl-phenyl, 3-(1-neo-pentyl-piperidin-3-yl)-5-trifluoromethyl-phenyl.

4. A compound of Formula (II) or a pharmaceutically acceptable salt, solvate, hydrate, cocrystal, or prodrug thereof, 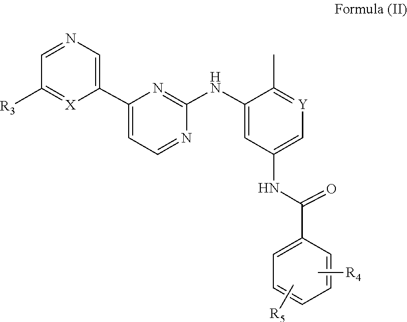
wherein X is CH or N;
R₃ is selected from the group consisting of hydrogen, halogen, (C1-C6)alkyl, amino, cyano, (C2-C8)alkynyl, mono(C1-C6)alkylamino, di(C1-C6)alkylamino, and (C1-C6)alkyoxy;
R₄ is hydrogen or CF₃;
R₅ is an unsubstituted or substituted alkylamine.

5. The compound of claim 4, wherein R₅ is selected from the group consisting of dimethylaminoethyl, diethylaminoethyl, dipropylaminoethyl, 2-(dimethylamino)propyl, 3-piperidinyl, 1-methyl-pyrrolidin-3-yl, 1-ethyl-pyrrolidin-3-yl, 1-propyl-pyrrolidin-3-yl, 1-isopropyl-pyrrolidin-3-yl, 1-iso-butyl-pyrrolidin-3-yl, t-butyl-pyrrolidin-3-yl, 1-neo-pentyl-pyrrolidin-3-yl, 1-methyl-piperidin-3-yl, 1-ethyl-piperidin-3-yl, 1-propyl-piperidin-3-yl, 1-butyl-piperidin-3-yl, 1-isopropyl-piperidin-3-yl, 1-iso-butyl-piperidin-3-yl, 1-t-butyl-piperidin-3-yl, 1-neo-pentyl-piperidin-3-yl, 1-methyl-piperidin-4-yl, 1-ethyl-piperidin-4-yl, 1-propyl-piperidin-4-yl, 1-isopropyl-piperidin-4-yl, 1-iso-butyl-piperidin-4-yl, 1-t-butyl)-piperidin-4-yl, 1-neo-pentyl-piperidin-4-yl, 4-methyl-piperazin-1-yl, 4-ethyl-piperazin-1-yl, 4-propyl-piperazin-1-yl, 4-isopropyl-piperazin-1-yl, 4-iso-butyl-piperazin-1-yl, 4-t-butyl-piperazin-1-yl, 4-neo-pentyl-piperazin-1-yl, and 1-methyl-azepan-3-yl, 1-ethyl-azepan-3-yl, 1-propyl-azepan-3-yl, 1-ethyl-azepan-3-yl, 4-(4-Methyl-piperazin-1-yl)-2-trifluoromethyl-phenyl, 4-(4-Ethyl-piperazin-1-yl)-2-trifluoromethyl-phenyl, 4-(4-Propyl-piperazin-1-yl)-2-trifluoromethyl-phenyl, 4-(4-Isopropyl-piperazin-1-yl)-2-trifluoromethyl-phenyl, 4-(4-Isobutyl-piperazin-1-yl)-2-trifluoromethyl-phenyl, 4-(4-Isobutyl-piperazin-1-yl)-2-trifluoromethyl-phenyl, 4-(4-t-Butyl-piperazin-1-yl)-2-trifluoromethyl-phenyl, 4-(4-neo-Pentyl-piperazin-1-yl)-2-trifluoromethyl-phenyl, 4-(1-Methyl-piperidin-4-yl)-2-trifluoromethyl-phenyl, 4-(1-Ethyl-piperidin-4-yl)-2-trifluoromethyl-phenyl, 4-(1-Propyl-piperidin-4-yl)-2-trifluoromethyl-phenyl, 4-(1-Isopropyl-piperidin-4-yl)-2-trifluoromethyl-phenyl, 4-(1-Isobutyl-piperidin-4-yl)-2-trifluoromethyl-phenyl, 4-(1-t-Butyl-piperidin-4-yl)-2-trifluoromethyl-phenyl, 4-(1-neo-pentyl-piperidin-4-yl)-2-trifluoromethyl-phenyl, 4-(1-Methyl-piperidin-3-yl)-2-trifluoromethyl-phenyl, 4-(1-Ethyl-piperidin-3-yl)-2-trifluoromethyl-phenyl, 4-(1-Propyl-piperidin-3-yl)-2-trifluoromethyl-phenyl, 4-(1-Isopropyl-piperidin-3-yl)-2-trifluoromethyl-phenyl, 4-(1-Isobutyl-piperidin-3-yl)-2-trifluoromethyl-phenyl, 4-(1-t-Butyl-piperidin-3-yl)-2-trifluoromethyl-phenyl, 4-(1-neo-pentyl-piperidin-3-yl)-2-trifluoromethyl-phenyl, 3-(4-Methyl-piperazin-1-yl)-5-trifluoromethyl-phenyl, 3-(4-Ethyl-piperazin-1-yl)-5-trifluoromethyl-phenyl, 3-(4-Propyl-piperazin-1-yl)-5-trifluoromethyl-phenyl, 3-(4-Isopropyl-piperazin-1-yl)-5-trifluoromethyl-phenyl, 3-(4-Isobutyl-piperazin-1-yl)-5-trifluoromethyl-phenyl, 3-(4-t-Butyl-piperazin-1-yl)-5-trifluoromethyl-phenyl, 3-(4-neo-pentyl-piperazin-1-yl)-5-trifluoromethyl-phenyl, 3-(1-Methyl-piperidin-4-yl)-5-trifluoromethyl-phenyl, 3-(1-Ethyl-piperidin-4-yl)-5-trifluoromethyl-phenyl, 3-(1-Propyl-piperidin-4-yl)-5-trifluoromethyl-phenyl, 3-(1-Isopropyl-piperidin-4-yl)-5-trifluoromethyl-phenyl, 3-(1-Isobutyl-piperidin-4-yl)-5-trifluoromethyl-phenyl, 3-(1-t-Butyl-piperidin-4-yl)-5-trifluoromethyl-phenyl, 3-(1-neo-pentyl-piperidin-4-yl)-5-trifluoromethyl-phenyl, 3-(1-Methyl-piperidin-3-yl)-5-trifluoromethyl-phenyl, 3-(1-Ethyl-piperidin-3-yl)-5-trifluoromethyl-phenyl, 3-(1-Propyl-piperidin-3-yl)-5-trifluoromethyl-phenyl, 3-(1-Isopropyl-piperidin-3-yl)-5-trifluoromethyl-phenyl, 3-(1-Isobutyl-piperidin-3-yl)-5-trifluoromethyl-phenyl, 3-(1-t-Butyl-piperidin-3-yl)-5-trifluoromethyl-phenyl, 3-(1-neo-pentyl-piperidin-3-yl)-5-trifluoromethyl-phenyl.

6. The compound of the claim 4 selected from the group consisting of:
4-(2-Diethylamino-ethyl)-N-[4-methyl-3-(4-pyridin-3-yl-pyrimidin-2-ylamino)-phenyl]-benzamide;
4-(2-Dipropylamino-ethyl)-N-[4-methyl-3-(4-pyridin-3-yl-pyrimidin-2-ylamino)-phenyl]-benzamide;
4-(2-Dimethylamino-propyl)-N-[4-methyl-3-(4-pyridin-3-yl-pyrimidin-2-ylamino)-phenyl]-benzamide;
N-[4-Methyl-3-(4-pyridin-3-yl-pyrimidin-2-ylamino)-phenyl]-4-(1-methyl-pyrrolidin-3-yl)-benzamide;
4-(1-Ethyl-pyrrolidin-3-yl)-N-[4-methyl-3-(4-pyridin-3-yl-pyrimidin-2-ylamino)-phenyl]-benzamide;
N-[4-Methyl-3-(4-pyridin-3-yl-pyrimidin-2-ylamino)-phenyl]-4-(1-propyl-pyrrolidin-3-yl)-benzamide;
4-(1-Ethyl-piperidin-3-yl)-N-[4-methyl-3-(4-pyridin-3-yl-pyrimidin-2-ylamino)-phenyl]-benzamide;
N-[4-Methyl-3-(4-pyridin-3-yl-pyrimidin-2-ylamino)-phenyl]-4-(1-propyl-piperidin-3-yl)-benzamide;
4-(1-Isopropyl-piperidin-3-yl)-N-[4-methyl-3-(4-pyridin-3-yl-pyrimidin-2-ylamino)-phenyl]-benzamide;
4-(1-Isobutyl-piperidin-3-yl)-N-[4-methyl-3-(4-pyridin-3-yl-pyrimidin-2-ylamino)-phenyl]-benzamide;
4-(1-tert-Butyl-piperidin-3-yl)-N-[4-methyl-3-(4-pyridin-3-yl-pyrimidin-2-ylamino)-phenyl]-benzamide;
4-[1-(2,2-Dimethyl-propyl)-piperidin-3-yl]-N-[4-methyl-3-(4-pyridin-3-yl-pyrimidin-2-ylamino)-phenyl]-benzamide;
4-(1-Ethyl-piperidin-4-yl)-N-[4-methyl-3-(4-pyridin-3-yl-pyrimidin-2-ylamino)-phenyl]-benzamide;
N-[4-Methyl-3-(4-pyridin-3-yl-pyrimidin-2-ylamino)-phenyl]-4-(1-propyl-piperidin-4-yl)-benzamide;
4-(1-Isopropyl-piperidin-4-yl)-N-[4-methyl-3-(4-pyridin-3-yl-pyrimidin-2-ylamino)-phenyl]-benzamide;
4-(1-Isobutyl-piperidin-4-yl)-N-[4-methyl-3-(4-pyridin-3-yl-pyrimidin-2-ylamino)-phenyl]-benzamide;
4-(1-tert-Butyl-piperidin-4-yl)-N-[4-methyl-3-(4-pyridin-3-yl-pyrimidin-2-ylamino)-phenyl]-benzamide;
4-[1-(2,2-Dimethyl-propyl)-piperidin-4-yl]-N-[4-methyl-3-(4-pyridin-3-yl-pyrimidin-2-ylamino)-phenyl]-benzamide;
4-(4-Ethyl-piperazin-1-yl)-N-[4-methyl-3-(4-pyridin-3-yl-pyrimidin-2-ylamino)-phenyl]-benzamide;
N-[4-Methyl-3-(4-pyridin-3-yl-pyrimidin-2-ylamino)-phenyl]-4-(4-propyl-piperazin-1-yl)-benzamide;
4-(4-Isopropyl-piperazin-1-yl)-N-[4-methyl-3-(4-pyridin-3-yl-pyrimidin-2-ylamino)-phenyl]-benzamide;
4-(4-Isobutyl-piperazin-1-yl)-N-[4-methyl-3-(4-pyridin-3-yl-pyrimidin-2-ylamino)-phenyl]-benzamide;
4-(4-tert-Butyl-piperazin-1-yl)-N-[4-methyl-3-(4-pyridin-3-yl-pyrimidin-2-ylamino)-phenyl]-benzamide;
4-[4-(2,2-Dimethyl-propyl)-piperazin-1-yl]-N-[4-methyl-3-(4-pyridin-3-yl-pyrimidin-2-ylamino)-phenyl]-benzamide;
4-(2-Dimethylamino-propyl)-N-[4-methyl-3-(4-pyridin-3-yl-pyrimidin-2-ylamino)-phenyl]-2-trifluoromethyl-benzamide;
N-[4-Methyl-3-(4-pyridin-3-yl-pyrimidin-2-ylamino)-phenyl]-4-(1-methyl-pyrrolidin-3-yl)-2-trifluoromethyl-benzamide;
4-(1-Methyl-piperidin-3-yl)-N-[4-methyl-3-(4-pyridin-3-yl-pyrimidin-2-ylamino)-phenyl]-2-trifluoromethyl-benzamide;
3-(1-Methyl-piperidin-3-yl)-N-[4-methyl-3-(4-pyridin-3-yl-pyrimidin-2-ylamino)-phenyl]-5-trifluoromethyl-benzamide;
N-[4-Methyl-3-(4-pyridin-3-yl-pyrimidin-2-ylamino)-phenyl]-3-(1-methyl-pyrrolidin-3-yl)-5-trifluoromethyl-benzamide;
4-(1-Ethyl-piperidin-3-yl)-N-[4-methyl-3-(4-pyridin-3-yl-pyrimidin-2-ylamino)-phenyl]-2-trifluoromethyl-benzamide;
N-[4-Methyl-3-(4-pyridin-3-yl-pyrimidin-2-ylamino)-phenyl]-4-(1-propyl-piperidin-3-yl)-2-trifluoromethyl-benzamide;
4-(1-Isopropyl-piperidin-3-yl)-N-[4-methyl-3-(4-pyridin-3-yl-pyrimidin-2-ylamino)-phenyl]-2-trifluoromethyl-benzamide;
4-(1-Methyl-piperidin-4-yl)-N-[4-methyl-3-(4-pyridin-3-yl-pyrimidin-2-ylamino)-phenyl]-2-trifluoromethyl-benzamide;
3-(1-Methyl-piperidin-4-yl)-N-[4-methyl-3-(4-pyridin-3-yl-pyrimidin-2-ylamino)-phenyl]-5-trifluoromethyl-benzamide;
4-(1-Ethyl-piperidin-4-yl)-N-[4-methyl-3-(4-pyridin-3-yl-pyrimidin-2-ylamino)-phenyl]-2-trifluoromethyl-benzamide;
N-[4-Methyl-3-(4-pyridin-3-yl-pyrimidin-2-ylamino)-phenyl]-4-(1-propyl-piperidin-4-yl)-2-trifluoromethyl-benzamide;
4-(1-Isopropyl-piperidin-4-yl)-N-[4-methyl-3-(4-pyridin-3-yl-pyrimidin-2-ylamino)-phenyl]-2-trifluoromethyl-benzamide;
4-(4-Ethyl-piperazin-1-yl)-N-[4-methyl-3-(4-pyridin-3-yl-pyrimidin-2-ylamino)-phenyl]-2-trifluoromethyl-benzamide;
N-[4-Methyl-3-(4-pyridin-3-yl-pyrimidin-2-ylamino)-phenyl]-4-(4-propyl-piperazin-1-yl)-2-trifluoromethyl-benzamide;
4-(4-Isopropyl-piperazin-1-yl)-N-[4-methyl-3-(4-pyridin-3-yl-pyrimidin-2-ylamino)-phenyl]-2-trifluoromethyl-benzamide;
4-(1-Methyl-azepan-3-yl)-N-[4-methyl-3-(4-pyridin-3-yl-pyrimidin-2-ylamino)-phenyl]-2-trifluoromethyl-benzamide;
4-(1-Methyl-azepan-3-yl)-N-[4-methyl-3-(4-pyrazin-2-yl-pyrimidin-2-ylamino)-phenyl]-2-trifluoromethyl-benzamide;
4-(1-Methyl-azepan-3-yl)-N-[4-methyl-3-(4-pyrazin-2-yl-pyrimidin-2-ylamino)-phenyl]-benzamide;
4-(1-Methyl-azepan-3-yl)-N-[4-methyl-3-(4-pyridin-3-yl-pyrimidin-2-ylamino)-phenyl]-benzamide;
4-(1-Methyl-azepan-3-yl)-N-(4-methyl-3-{4-[5-(1-methyl-1H-pyrazol-3-yl)-pyridin-3-yl]-pyrimidin-2-ylamino}-phenyl)-benzamide;
N-(4-Methyl-3-{4-[5-(1-methyl-1H-pyrazol-3-yl)-pyridin-3-yl]-pyrimidin-2-ylamino}-phenyl)-4-(1-methyl-piperidin-3-yl)-benzamide;
N-(4-Methyl-3-{4-[5-(1-methyl-1H-pyrazol-3-yl)-pyridin-3-yl]-pyrimidin-2-ylamino}-phenyl)-4-(1-methyl-piperidin-4-yl)-benzamide;
N-(4-Methyl-3-{4-[5-(1-methyl-1H-pyrazol-3-yl)-pyridin-3-yl]-pyrimidin-2-ylamino}-phenyl)-4-(4-methyl-piperazin-1-yl)-benzamide;
N-(4-Methyl-3-{4-[5-(4-methyl-isoxazol-5-yl)-pyridin-3-yl]-pyrimidin-2-ylamino}-phenyl)-4-(4-methyl-piperazin-1-yl)-benzamide;
N-(4-Methyl-3-{4-[5-(4-methyl-isoxazol-5-yl)-pyridin-3-yl]-pyrimidin-2-ylamino}-phenyl)-4-(4-methyl-piperazin-1-yl)-2-trifluoromethyl-benzamide;
N-(4-Methyl-3-{4-[5-(4-methyl-isoxazol-5-yl)-pyridin-3-yl]-pyrimidin-2-ylamino}-phenyl)-4-(1-methyl-piperidin-4-yl)-2-trifluoromethyl-benzamide;
N-(4-Methyl-3-{4-[5-(4-methyl-isoxazol-5-yl)-pyridin-3-yl]-pyrimidin-2-ylamino}-phenyl)-4-(1-methyl-piperidin-4-yl)-benzamide;
N-(4-Methyl-3-{4-[5-(4-methyl-isoxazol-5-yl)-pyridin-3-yl]-pyrimidin-2-ylamino}-phenyl)-4-(1-methyl-piperidin-3-yl)-benzamide;
N-(4-Methyl-3-{4-[5-(4-methyl-isoxazol-5-yl)-pyridin-3-yl]-pyrimidin-2-ylamino}-phenyl)-4-(1-methyl-piperidin-3-yl)-2-trifluoromethyl-benzamide;
4-(1-Methyl-azepan-3-yl)-N-(4-methyl-3-{4-[5-(4-methyl-isoxazol-5-yl)-pyridin-3-yl]-pyrimidin-2-ylamino}-phenyl)-2-trifluoromethyl-benzamide;
4-(1-Methyl-azepan-3-yl)-N-(4-methyl-3-{4-[5-(4-methyl-isoxazol-5-yl)-pyridin-3-yl]-pyrimidin-2-ylamino}-phenyl)-benzamide;
4-(2-Dimethylamino-ethyl)-N-(4-methyl-3-{4-[5-(4-methyl-isoxazol-5-yl)-pyridin-3-yl]-pyrimidin-2-ylamino}-phenyl)-benzamide;
4-(2-Dimethylamino-ethyl)-N-(4-methyl-3-{4-[5-(4-methyl-isoxazol-5-yl)-pyridin-3-yl]-pyrimidin-2-ylamino}-phenyl)-2-trifluoromethyl-benzamide;
4-(2-Dimethylamino-propyl)-N-(4-methyl-3-{4-[5-(4-methyl-isoxazol-5-yl)-pyridin-3-yl]-pyrimidin-2-ylamino}-phenyl)-benzamide;
4-(2-Dimethylamino-propyl)-N-(4-methyl-3-{4-[5-(4-methyl-isoxazol-5-yl)-pyridin-3-yl]-pyrimidin-2-ylamino}-phenyl)-2-trifluoromethyl-benzamide;
N-(4-Methyl-3-{4-[5-(4-methyl-isoxazol-5-yl)-pyridin-3-yl]-pyrimidin-2-ylamino}-phenyl)-4-(1-methyl-pyrrolidin-3-yl)-benzamide;
N-(4-Methyl-3-{4-[5-(4-methyl-isoxazol-5-yl)-pyridin-3-yl]-pyrimidin-2-ylamino}-phenyl)-4-(1-methyl-pyrrolidin-3-yl)-2-trifluoromethyl-benzamide;
N-(4-Methyl-3-{4-[5-(4-methyl-isoxazol-5-yl)-pyridin-3-yl]-pyrimidin-2-ylamino}-phenyl)-3-(1-methyl-pyrrolidin-3-yl)-5-trifluoromethyl-benzamide;
4-(1-Ethyl-piperidin-3-yl)-N-(4-methyl-3-{4-[5-(4-methyl-isoxazol-5-yl)-pyridin-3-yl]-pyrimidin-2-ylamino}-phenyl)-benzamide;
4-(1-Ethyl-piperidin-3-yl)-N-(4-methyl-3-{4-[5-(4-methyl-isoxazol-5-yl)-pyridin-3-yl]-pyrimidin-2-ylamino}-phenyl)-2-trifluoromethyl-benzamide;
4-(2-Dimethylamino-ethyl)-N-[6-methyl-5-(4-pyridin-3-yl-pyrimidin-2-ylamino)-pyridin-3-yl]-benzamide;
4-(2-Dimethylamino-propyl)-N-[6-methyl-5-(4-pyridin-3-yl-pyrimidin-2-ylamino)-pyridin-3-yl]-benzamide;
4-(2-Diethylamino-ethyl)-N-[6-methyl-5-(4-pyridin-3-yl-pyrimidin-2-ylamino)-pyridin-3-yl]-benzamide;
4-(2-Dipropylamino-ethyl)-N-[6-methyl-5-(4-pyridin-3-yl-pyrimidin-2-ylamino)-pyridin-3-yl]-benzamide;
N-[6-Methyl-5-(4-pyridin-3-yl-pyrimidin-2-ylamino)-pyridin-3-yl]-4-pyrrolidin-3-yl-benzamide;
N-[6-Methyl-5-(4-pyridin-3-yl-pyrimidin-2-ylamino)-pyridin-3-yl]-4-(1-methyl-pyrrolidin-3-yl)-benzamide;
4-(1-Ethyl-pyrrolidin-3-yl)-N-[6-methyl-5-(4-pyridin-3-yl-pyrimidin-2-ylamino)-pyridin-3-yl]-benzamide;
N-[6-Methyl-5-(4-pyridin-3-yl-pyrimidin-2-ylamino)-pyridin-3-yl]-4-(1-propyl-pyrrolidin-3-yl)-benzamide;
4-(1-Isopropyl-pyrrolidin-3-yl)-N-[6-methyl-5-(4-pyridin-3-yl-pyrimidin-2-ylamino)-pyridin-3-yl]-benzamide;
4-(1-Isobutyl-pyrrolidin-3-yl)-N-[6-methyl-5-(4-pyridin-3-yl-pyrimidin-2-ylamino)-pyridin-3-yl]-benzamide;
4-[1-(2,2-Dimethyl-propyl)-pyrrolidin-3-yl]-N-[6-methyl-5-(4-pyridin-3-yl-pyrimidin-2-ylamino)-pyridin-3-yl]-benzamide;
N-[6-Methyl-5-(4-pyridin-3-yl-pyrimidin-2-ylamino)-pyridin-3-yl]-4-piperidin-3-yl-benzamide;
4-(1-Methyl-piperidin-3-yl)-N-[6-methyl-5-(4-pyridin-3-yl-pyrimidin-2-ylamino)-pyridin-3-yl]-benzamide;
4-(1-Methyl-piperidin-3-yl)-N-[6-methyl-5-(4-pyridin-3-yl-pyrimidin-2-ylamino)-pyridin-3-yl]-2-trifluoromethyl-benzamide;
4-(1-Ethyl-piperidin-3-yl)-N-[6-methyl-5-(4-pyridin-3-yl-pyrimidin-2-ylamino)-pyridin-3-yl]-2-trifluoromethyl-benzamide;
4-(1-Ethyl-piperidin-3-yl)-N-[6-methyl-5-(4-pyridin-3-yl-pyrimidin-2-ylamino)-pyridin-3-yl]-benzamide;
N-[6-Methyl-5-(4-pyridin-3-yl-pyrimidin-2-ylamino)-pyridin-3-yl]-4-(1-propyl-piperidin-3-yl)-benzamide;
N-[6-Methyl-5-(4-pyridin-3-yl-pyrimidin-2-ylamino)-pyridin-3-yl]-4-(1-propyl-piperidin-3-yl)-2-trifluoromethyl-benzamide;
N-[6-Methyl-5-(4-pyridin-3-yl-pyrimidin-2-ylamino)-pyridin-3-yl]-4-piperidin-4-yl-benzamide;
4-(1-Methyl-piperidin-4-yl)-N-[6-methyl-5-(4-pyridin-3-yl-pyrimidin-2-ylamino)-pyridin-3-yl]-benzamide;
4-(1-Methyl-piperidin-4-yl)-N-[6-methyl-5-(4-pyridin-3-yl-pyrimidin-2-ylamino)-pyridin-3-yl]-2-trifluoromethyl-benzamide;
4-(1-Ethyl-piperidin-4-yl)-N-[6-methyl-5-(4-pyridin-3-yl-pyrimidin-2-ylamino)-pyridin-3-yl]-benzamide;
4-(1-Ethyl-piperidin-4-yl)-N-[6-methyl-5-(4-pyridin-3-yl-pyrimidin-2-ylamino)-pyridin-3-yl]-2-trifluoromethyl-benzamide;
N-[6-Methyl-5-(4-pyridin-3-yl-pyrimidin-2-ylamino)-pyridin-3-yl]-4-(1-propyl-piperidin-4-yl)-benzamide;
N-[6-Methyl-5-(4-pyridin-3-yl-pyrimidin-2-ylamino)-pyridin-3-yl]-4-(1-propyl-piperidin-4-yl)-2-trifluoromethyl-benzamide;
N-[6-Methyl-5-(4-pyridin-3-yl-pyrimidin-2-ylamino)-pyridin-3-yl]-4-piperazin-1-yl-benzamide;
4-(4-Methyl-piperazin-1-yl)-N-[6-methyl-5-(4-pyridin-3-yl-pyrimidin-2-ylamino)-pyridin-3-yl]-benzamide;
4-(4-Ethyl-piperazin-1-yl)-N-[6-methyl-5-(4-pyridin-3-yl-pyrimidin-2-ylamino)-pyridin-3-yl]-benzamide;
N-[6-Methyl-5-(4-pyridin-3-yl-pyrimidin-2-ylamino)-pyridin-3-yl]-4-(4-propyl-piperazin-1-yl)-benzamide;
N-[6-Methyl-5-(4-pyridin-3-yl-pyrimidin-2-ylamino)-pyridin-3-yl]-4-(4-propyl-piperazin-1-yl)-2-trifluoromethyl-benzamide;
4-(4-Ethyl-piperazin-1-yl)-N-[6-methyl-5-(4-pyridin-3-yl-pyrimidin-2-ylamino)-pyridin-3-yl]-2-trifluoromethyl-benzamide;
4-(4-Methyl-piperazin-1-yl)-N-[6-methyl-5-(4-pyridin-3-yl-pyrimidin-2-ylamino)-pyridin-3-yl]-2-trifluoromethyl-benzamide;
3-(4-Methyl-piperazin-1-yl)-N-[6-methyl-5-(4-pyridin-3-yl-pyrimidin-2-ylamino)-pyridin-3-yl]-5-trifluoromethyl-benzamide;
3-(4-Ethyl-piperazin-1-yl)-N-[6-methyl-5-(4-pyridin-3-yl-pyrimidin-2-ylamino)-pyridin-3-yl]-5-trifluoromethyl-benzamide;
N-[6-Methyl-5-(4-pyridin-3-yl-pyrimidin-2-ylamino)-pyridin-3-yl]-3-(4-propyl-piperazin-1-yl)-5-trifluoromethyl-benzamide;
N-[6-Methyl-5-(4-pyridin-3-yl-pyrimidin-2-ylamino)-pyridin-3-yl]-3-(1-propyl-piperidin-4-yl)-5-trifluoromethyl-benzamide;
3-(1-Ethyl-piperidin-4-yl)-N-[6-methyl-5-(4-pyridin-3-yl-pyrimidin-2-ylamino)-pyridin-3-yl]-5-trifluoromethyl-benzamide;
3-(1-Methyl-piperidin-4-yl)-N-[6-methyl-5-(4-pyridin-3-yl-pyrimidin-2-ylamino)-pyridin-3-yl]-5-trifluoromethyl-benzamide;
3-(1-Methyl-piperidin-3-yl)-N-[6-methyl-5-(4-pyridin-3-yl-pyrimidin-2-ylamino)-pyridin-3-yl]-5-trifluoromethyl-benzamide;
3-(1-Ethyl-piperidin-3-yl)-N-[6-methyl-5-(4-pyridin-3-yl-pyrimidin-2-ylamino)-pyridin-3-yl]-5-trifluoromethyl-benzamide;
N-[6-Methyl-5-(4-pyridin-3-yl-pyrimidin-2-ylamino)-pyridin-3-yl]-3-(1-propyl-piperidin-3-yl)-5-trifluoromethyl-benzamide; and a pharmaceutically acceptable salt, solvate, hydrate, cocrystal, or prodrug thereof.

7. A compound of Formula (III) or a pharmaceutically acceptable salt, solvate, hydrate, cocrystal, or prodrug thereof, 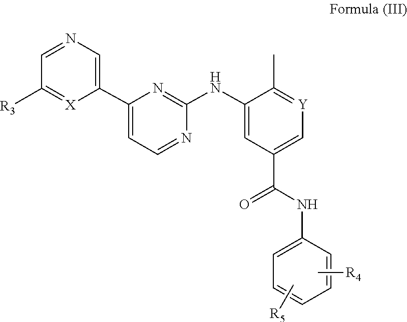
wherein X is CH or N;
Y is CH or N;
R₃ is selected from the group consisting of hydrogen, halogen, (C1-C6)alkyl, amino, cyano, (C2-C8)alkynyl, mono(C1-C6)alkylamino, di(C1-C6)alkylamino, and (C1-C6)alkyoxy;
R₄ is hydrogen or CF₃;
R₅ is an unsubstituted or substituted alkylamine.

8. The compound of claim 7, wherein R₅ is selected from the group consisting of dimethylaminoethyl, diethylaminoethyl, dipropylaminoethyl, 2-(dimethylamino)propyl, 3-piperidinyl, 1-methyl-pyrrolidin-3-yl, 1-ethyl-pyrrolidin-3-yl, 1-propyl-pyrrolidin-3-yl, 1-isopropyl-pyrrolidin-3-yl, 1-iso-butyl-pyrrolidin-3-yl, t-butyl-pyrrolidin-3-yl, 1-neo-pentyl-pyrrolidin-3-yl, 1-methyl-piperidin-3-yl, 1-ethyl-piperidin-3-yl, 1-propyl-piperidin-3-yl, 1-butyl-piperidin-3-yl, 1-isopropyl-piperidin-3-yl, 1-iso-butyl-piperidin-3-yl, 1-t-butyl-piperidin-3-yl, 1-neo-pentyl-piperidin-3-yl, 1-methyl-piperidin-4-yl, 1-ethyl-piperidin-4-yl, 1-propyl-piperidin-4-yl, 1-isopropyl-piperidin-4-yl, 1-iso-butyl-piperidin-4-yl, 1-t-butyl)-piperidin-4-yl, 1-neo-pentyl-piperidin-4-yl, 4-methyl-piperazin-1-yl, 4-ethyl-piperazin-1-yl, 4-propyl-piperazin-1-yl, 4-isopropyl-piperazin-1-yl, 4-iso-butyl-piperazin-1-yl, 4-t-butyl-piperazin-1-yl, 4-neo-pentyl-piperazin-1-yl, and 1-methyl-azepan-3-yl, 1-ethyl-azepan-3-yl, 1-propyl-azepan-3-yl, 1-ethyl-azepan-3-yl, 4-(4-Methyl-piperazin-1-yl)-2-trifluoromethyl-phenyl, 4-(4-Ethyl-piperazin-1-yl)-2-trifluoromethyl-phenyl, 4-(4-Propyl-piperazin-1-yl)-2-trifluoromethyl-phenyl, 4-(4-Isopropyl-piperazin-1-yl)-2-trifluoromethyl-phenyl, 4-(4-Isobutyl-piperazin-1-yl)-2-trifluoromethyl-phenyl, 4-(4-Isobutyl-piperazin-1-yl)-2-trifluoromethyl-phenyl, 4-(4-t-Butyl-piperazin-1-yl)-2-trifluoromethyl-phenyl, 4-(4-neo-Pentyl-piperazin-1-yl)-2-trifluoromethyl-phenyl, 4-(1-Methyl-piperidin-4-yl)-2-trifluoromethyl-phenyl, 4-(1-Ethyl-piperidin-4-yl)-2-trifluoromethyl-phenyl, 4-(1-Propyl-piperidin-4-yl)-2-trifluoromethyl-phenyl, 4-(1-Isopropyl-piperidin-4-yl)-2-trifluoromethyl-phenyl, 4-(1-Isobutyl-piperidin-4-yl)-2-trifluoromethyl-phenyl, 4-(1-t-Butyl-piperidin-4-yl)-2-trifluoromethyl-phenyl, 4-(1-neo-pentyl-piperidin-4-yl)-2-trifluoromethyl-phenyl, 4-(1-Methyl-piperidin-3-yl)-2-trifluoromethyl-phenyl, 4-(1-Ethyl-piperidin-3-yl)-2-trifluoromethyl-phenyl, 4-(1-Propyl-piperidin-3-yl)-2-trifluoromethyl-phenyl, 4-(1-Isopropyl-piperidin-3-yl)-2-trifluoromethyl-phenyl, 4-(1-Isobutyl-piperidin-3-yl)-2-trifluoromethyl-phenyl, 4-(1-t-Butyl-piperidin-3-yl)-2-trifluoromethyl-phenyl, 4-(1-neo-pentyl-piperidin-3-yl)-2-trifluoromethyl-phenyl, 3-(4-Methyl-piperazin-1-yl)-5-trifluoromethyl-phenyl, 3-(4-Ethyl-piperazin-1-yl)-5-trifluoromethyl-phenyl, 3-(4-Propyl-piperazin-1-yl)-5-trifluoromethyl-phenyl, 3-(4-Isopropyl-piperazin-1-yl)-5-trifluoromethyl-phenyl, 3-(4-Isobutyl-piperazin-1-yl)-5-trifluoromethyl-phenyl, 3-(4-t-Butyl-piperazin-1-yl)-5-trifluoromethyl-phenyl, 3-(4-neo-pentyl-piperazin-1-yl)-5-trifluoromethyl-phenyl, 3-(1-Methyl-piperidin-4-yl)-5-trifluoromethyl-phenyl, 3-(1-Ethyl-piperidin-4-yl)-5-trifluoromethyl-phenyl, 3-(1-Propyl-piperidin-4-yl)-5-trifluoromethyl-phenyl, 3-(1-Isopropyl-piperidin-4-yl)-5-trifluoromethyl-phenyl, 3-(1-Isobutyl-piperidin-4-yl)-5-trifluoromethyl-phenyl, 3-(1-t-Butyl-piperidin-4-yl)-5-trifluoromethyl-phenyl, 3-(1-neo-pentyl-piperidin-4-yl)-5-trifluoromethyl-phenyl, 3-(1-Methyl-piperidin-3-yl)-5-trifluoromethyl-phenyl, 3-(1-Ethyl-piperidin-3-yl)-5-trifluoromethyl-phenyl, 3-(1-Propyl-piperidin-3-yl)-5-trifluoromethyl-phenyl, 3-(1-Isopropyl-piperidin-3-yl)-5-trifluoromethyl-phenyl, 3-(1-Isobutyl-piperidin-3-yl)-5-trifluoromethyl-phenyl, 3-(1-t-Butyl-piperidin-3-yl)-5-trifluoromethyl-phenyl, 3-(1-neo-pentyl-piperidin-3-yl)-5-trifluoromethyl-phenyl.

9. The compound of the claim 7 selected from the group consisting of:
N-[4-(2-Dimethylamino-ethyl)-phenyl]-4-methyl-3-(4-pyridin-3-yl-pyrimidin-2-ylamino)-benzamide;
4-Methyl-N-[4-(1-methyl-pyrrolidin-3-yl)-phenyl]-3-(4-pyridin-3-yl-pyrimidin-2-ylamino)-benzamide;
N-[4-(1-Ethyl-pyrrolidin-3-yl)-phenyl]-4-methyl-3-(4-pyridin-3-yl-pyrimidin-2-ylamino)-benzamide;
4-Methyl-N-[4-(1-propyl-pyrrolidin-3-yl)-phenyl]-3-(4-pyridin-3-yl-pyrimidin-2-ylamino)-benzamide;
N-[4-(2-Dimethylamino-propyl)-phenyl]-4-methyl-3-(4-pyridin-3-yl-pyrimidin-2-ylamino)-benzamide;
N-[4-(2-Diethylamino-ethyl)-phenyl]-4-methyl-3-(4-pyridin-3-yl-pyrimidin-2-ylamino)-benzamide;
N-[4-(2-Propylamino-ethyl)-phenyl]-4-methyl-3-(4-pyridin-3-yl-pyrimidin-2-ylamino)-benzamide;
4-Methyl-N-(4-piperidin-3-yl-phenyl)-3-(4-pyridin-3-yl-pyrimidin-2-ylamino)-benzamide;
4-Methyl-3-(4-pyridin-3-yl-pyrimidin-2-ylamino)-N-(4-pyrrolidin-3-yl-phenyl)-benzamide;
4-Methyl-N-[4-(1-methyl-piperidin-3-yl)-phenyl]-3-(4-pyridin-3-yl-pyrimidin-2-ylamino)-benzamide;
N-[4-(1-Ethyl-piperidin-3-yl)-phenyl]-4-methyl-3-(4-pyridin-3-yl-pyrimidin-2-ylamino)-benzamide;
4-Methyl-N-[4-(1-propyl-piperidin-3-yl)-phenyl]-3-(4-pyridin-3-yl-pyrimidin-2-ylamino)-benzamide;
N-[4-(1-Isopropyl-piperidin-3-yl)-phenyl]-4-methyl-3-(4-pyridin-3-yl-pyrimidin-2-ylamino)-benzamide;
N-[4-(1-tert-Butyl-piperidin-3-yl)-phenyl]-4-methyl-3-(4-pyridin-3-yl-pyrimidin-2-ylamino)-benzamide;
N-[4-(1-Isobutyl-piperidin-3-yl)-phenyl]-4-methyl-3-(4-pyridin-3-yl-pyrimidin-2-ylamino)-benzamide;
N-{4-[1-(2,2-Dimethyl-propyl)-piperidin-3-yl]-phenyl}-4-methyl-3-(4-pyridin-3-yl-pyrimidin-2-ylamino)-benzamide;
4-Methyl-N-(4-piperidin-4-yl-phenyl)-3-(4-pyridin-3-yl-pyrimidin-2-ylamino)-benzamide;
4-Methyl-N-[4-(1-methyl-piperidin-4-yl)-phenyl]-3-(4-pyridin-3-yl-pyrimidin-2-ylamino)-benzamide;
N-[4-(1-Ethyl-piperidin-4-yl)-phenyl]-4-methyl-3-(4-pyridin-3-yl-pyrimidin-2-ylamino)-benzamide;
4-Methyl-N-[4-(1-propyl-piperidin-4-yl)-phenyl]-3-(4-pyridin-3-yl-pyrimidin-2-ylamino)-benzamide;
N-[4-(1-Isopropyl-piperidin-4-yl)-phenyl]-4-methyl-3-(4-pyridin-3-yl-pyrimidin-2-ylamino)-benzamide;
N-[4-(1-tert-Butyl-piperidin-4-yl)-phenyl]-4-methyl-3-(4-pyridin-3-yl-pyrimidin-2-ylamino)-benzamide;
N-[4-(1-Isobutyl-piperidin-4-yl)-phenyl]-4-methyl-3-(4-pyridin-3-yl-pyrimidin-2-ylamino)-benzamide;
N-{4-[1-(2,2-Dimethyl-propyl)-piperidin-4-yl]-phenyl}-4-methyl-3-(4-pyridin-3-yl-pyrimidin-2-ylamino)-benzamide;
N-{4-[4-(2,2-Dimethyl-propyl)-piperazin-1-yl]-phenyl}-4-methyl-3-(4-pyridin-3-yl-pyrimidin-2-ylamino)-benzamide;
N-[4-(4-Isobutyl-piperazin-1-yl)-phenyl]-4-methyl-3-(4-pyridin-3-yl-pyrimidin-2-ylamino)-benzamide;
4-Methyl-N-[4-(4-propyl-piperazin-1-yl)-phenyl]-3-(4-pyridin-3-yl-pyrimidin-2-ylamino)-benzamide;
N-[4-(4-Ethyl-piperazin-1-yl)-phenyl]-4-methyl-3-(4-pyridin-3-yl-pyrimidin-2-ylamino)-benzamide;
4-Methyl-N-[4-(4-methyl-piperazin-1-yl)-phenyl]-3-(4-pyridin-3-yl-pyrimidin-2-ylamino)-benzamide;
4-Methyl-N-(4-piperazin-1-yl-phenyl)-3-(4-pyridin-3-yl-pyrimidin-2-ylamino)-benzamide;
N-(4-Azepan-3-yl-phenyl)-4-methyl-3-(4-pyridin-3-yl-pyrimidin-2-ylamino)-benzamide;
4-Methyl-N-[4-(1-methyl-azepan-3-yl)-phenyl]-3-(4-pyridin-3-yl-pyrimidin-2-ylamino)-benzamide;
N-[4-(1-Ethyl-azepan-3-yl)-phenyl]-4-methyl-3-(4-pyridin-3-yl-pyrimidin-2-ylamino)-benzamide;
4-Methyl-N-[4-(1-propyl-azepan-3-yl)-phenyl]-3-(4-pyridin-3-yl-pyrimidin-2-ylamino)-benzamide;
N-[4-(1-Isopropyl-azepan-3-yl)-phenyl]-4-methyl-3-(4-pyridin-3-yl-pyrimidin-2-ylamino)-benzamide;
N-[4-(1-Isobutyl-azepan-3-yl)-phenyl]-4-methyl-3-(4-pyridin-3-yl-pyrimidin-2-ylamino)-benzamide;
N-{4-[1-(2,2-Dimethyl-propyl)-azepan-3-yl]-phenyl}-4-methyl-3-(4-pyridin-3-yl-pyrimidin-2-ylamino)-benzamide;
N-[4-(1-tert-Butyl-azepan-3-yl)-phenyl]-4-methyl-3-(4-pyridin-3-yl-pyrimidin-2-ylamino)-benzamide;
4-Methyl-N-[4-(1-methyl-azepan-3-yl)-2-trifluoromethyl-phenyl]-3-(4-pyridin-3-yl-pyrimidin-2-ylamino)-benzamide;
4-Methyl-N-[4-(1-methyl-piperidin-3-yl)-2-trifluoromethyl-phenyl]-3-(4-pyridin-3-yl-pyrimidin-2-ylamino)-benzamide;
4-Methyl-N-[4-(1-methyl-piperidin-4-yl)-2-trifluoromethyl-phenyl]-3-(4-pyridin-3-yl-pyrimidin-2-ylamino)-benzamide;
4-Methyl-N-[4-(4-methyl-piperazin-1-yl)-2-trifluoromethyl-phenyl]-3-(4-pyridin-3-yl-pyrimidin-2-ylamino)-benzamide;
4-Methyl-N-[4-(4-methyl-piperazin-1-yl)-2-trifluoromethyl-phenyl]-3-(4-pyrazin-2-yl-pyrimidin-2-ylamino)-benzamide;
4-Methyl-N-[4-(1-methyl-piperidin-4-yl)-2-trifluoromethyl-phenyl]-3-(4-pyrazin-2-yl-pyrimidin-2-ylamino)-benzamide;
4-Methyl-N-[4-(1-methyl-piperidin-3-yl)-2-trifluoromethyl-phenyl]-3-(4-pyrazin-2-yl-pyrimidin-2-ylamino)-benzamide;
4-Methyl-N-[4-(1-methyl-azepan-3-yl)-2-trifluoromethyl-phenyl]-3-(4-pyrazin-2-yl-pyrimidin-2-ylamino)-benzamide;
4-Methyl-N-[4-(1-methyl-azepan-3-yl)-2-trifluoromethyl-phenyl]-3-{4-[5-(1-methyl-1H-pyrazol-3-yl)-pyridin-3-yl]-pyrimidin-2-ylamino}-benzamide;
4-Methyl-N-[4-(1-methyl-piperidin-3-yl)-2-trifluoromethyl-phenyl]-3-{4-[5-(1-methyl-1H-pyrazol-3-yl)-pyridin-3-yl]-pyrimidin-2-ylamino}-benzamide;
4-Methyl-N-[4-(1-methyl-piperidin-4-yl)-2-trifluoromethyl-phenyl]-3-{4-[5-(1-methyl-1H-pyrazol-3-yl)-pyridin-3-yl]-pyrimidin-2-ylamino}-benzamide;
4-Methyl-N-[4-(4-methyl-piperazin-1-yl)-2-trifluoromethyl-phenyl]-3-{4-[5-(1-methyl-1H-pyrazol-3-yl)-pyridin-3-yl]-pyrimidin-2-ylamino}-benzamide;
4-Methyl-N-[4-(4-methyl-piperazin-1-yl)-phenyl]-3-{4-[5-(1-methyl-1H-pyrazol-3-yl)-pyridin-3-yl]-pyrimidin-2-ylamino}-benzamide;
4-Methyl-N-[4-(1-methyl-piperidin-4-yl)-phenyl]-3-{4-[5-(1-methyl-1H-pyrazol-3-yl)-pyridin-3-yl]-pyrimidin-2-ylamino}-benzamide;
4-Methyl-N-[4-(1-methyl-piperidin-3-yl)-phenyl]-3-{4-[5-(1-methyl-1H-pyrazol-3-yl)-pyridin-3-yl]-pyrimidin-2-ylamino}-benzamide;
4-Methyl-N-[4-(1-methyl-azepan-3-yl)-phenyl]-3-{4-[5-(1-methyl-1H-pyrazol-3-yl)-pyridin-3-yl]-pyrimidin-2-ylamino}-benzamide;
4-Methyl-N-[4-(1-methyl-azepan-3-yl)-phenyl]-3-{4-[5-(4-methyl-isoxazol-5-yl)-pyridin-3-yl]-pyrimidin-2-ylamino}-benzamide;
4-Methyl-3-{4-[5-(4-methyl-isoxazol-5-yl)-pyridin-3-yl]-pyrimidin-2-ylamino}-N-[4-(1-methyl-piperidin-3-yl)-phenyl]-benzamide;
4-Methyl-3-{4-[5-(4-methyl-isoxazol-5-yl)-pyridin-3-yl]-pyrimidin-2-ylamino}-N-[4-(1-methyl-piperidin-3-yl)-2-trifluoromethyl-phenyl]-benzamide;
4-Methyl-3-{4-[5-(4-methyl-isoxazol-5-yl)-pyridin-3-yl]-pyrimidin-2-ylamino}-N-[4-(1-methyl-piperidin-4-yl)-2-trifluoromethyl-phenyl]-benzamide;
4-Methyl-3-{4-[5-(4-methyl-isoxazol-5-yl)-pyridin-3-yl]-pyrimidin-2-ylamino}-N-[4-(1-methyl-pyrrolidin-3-yl)-5-trifluoromethyl-phenyl]-benzamide;
4-Methyl-3-{4-[5-(4-methyl-isoxazol-5-yl)-pyridin-3-yl]-pyrimidin-2-ylamino}-N-[4-(1-methyl-pyrrolidin-3-yl)-2-trifluoromethyl-phenyl]-benzamide;
4-Methyl-3-{4-[5-(4-methyl-isoxazol-5-yl)-pyridin-3-yl]-pyrimidin-2-ylamino}-N-[4-(1-methyl-piperidin-4-yl)-phenyl]-benzamide;
4-Methyl-3-{4-[5-(4-methyl-isoxazol-5-yl)-pyridin-3-yl]-pyrimidin-2-ylamino}-N-[4-(4-methyl-piperazin-1-yl)-phenyl]-benzamide;
4-Methyl-3-{4-[5-(4-methyl-isoxazol-5-yl)-pyridin-3-yl]-pyrimidin-2-ylamino}-N-[4-(4-methyl-piperazin-1-yl)-2-trifluoromethyl-phenyl]-benzamide;
N-[4-(2-Dimethylamino-ethyl)-2-trifluoromethyl-phenyl]-4-methyl-3-{4-[5-(4-methyl-isoxazol-5-yl)-pyridin-3-yl]-pyrimidin-2-ylamino}-benzamide;
N-[4-(2-Dimethylamino-ethyl)-phenyl]-4-methyl-3-{4-[5-(4-methyl-isoxazol-5-yl)-pyridin-3-yl]-pyrimidin-2-ylamino}-benzamide;
N-[4-(2-Dimethylamino-propyl)-phenyl]-4-methyl-3-{4-[5-(4-methyl-isoxazol-5-yl)-pyridin-3-yl]-pyrimidin-2-ylamino}-benzamide;
N-[4-(2-Dimethylamino-propyl)-2-trifluoromethyl-phenyl]-4-methyl-3-{4-[5-(4-methyl-isoxazol-5-yl)-pyridin-3-yl]-pyrimidin-2-ylamino}-benzamide;
N-[4-(4-Ethyl-piperazin-1-yl)-2-trifluoromethyl-phenyl]-4-methyl-3-(4-pyridin-3-yl-pyrimidin-2-ylamino)-benzamide;
4-Methyl-N-[4-(4-propyl-piperazin-1-yl)-2-trifluoromethyl-phenyl]-3-(4-pyridin-3-yl-pyrimidin-2-ylamino)-benzamide;
4-Methyl-N-[4-(1-propyl-piperidin-4-yl)-2-trifluoromethyl-phenyl]-3-(4-pyridin-3-yl-pyrimidin-2-ylamino)-benzamide;
N-[4-(1-Ethyl-piperidin-4-yl)-2-trifluoromethyl-phenyl]-4-methyl-3-(4-pyridin-3-yl-pyrimidin-2-ylamino)-benzamide;
N-[4-(1-Ethyl-piperidin-3-yl)-2-trifluoromethyl-phenyl]-4-methyl-3-(4-pyridin-3-yl-pyrimidin-2-ylamino)-benzamide;
4-Methyl-N-[4-(1-propyl-piperidin-3-yl)-2-trifluoromethyl-phenyl]-3-(4-pyridin-3-yl-pyrimidin-2-ylamino)-benzamide;
4-Methyl-N-[3-(1-propyl-piperidin-3-yl)-5-trifluoromethyl-phenyl]-3-(4-pyridin-3-yl-pyrimidin-2-ylamino)-benzamide;
N-[3-(1-Ethyl-piperidin-3-yl)-5-trifluoromethyl-phenyl]-4-methyl-3-(4-pyridin-3-yl-pyrimidin-2-ylamino)-benzamide;
N-[3-(1-Ethyl-piperidin-4-yl)-5-trifluoromethyl-phenyl]-4-methyl-3-(4-pyridin-3-yl-pyrimidin-2-ylamino)-benzamide;
4-Methyl-N-[3-(1-propyl-piperidin-4-yl)-5-trifluoromethyl-phenyl]-3-(4-pyridin-3-yl-pyrimidin-2-ylamino)-benzamide;
4-Methyl-N-[3-(4-propyl-piperazin-1-yl)-5-trifluoromethyl-phenyl]-3-(4-pyridin-3-yl-pyrimidin-2-ylamino)-benzamide;
N-[3-(4-Ethyl-piperazin-1-yl)-5-trifluoromethyl-phenyl]-4-methyl-3-(4-pyridin-3-yl-pyrimidin-2-ylamino)-benzamide;
N-[4-(2-Dimethylamino-ethyl)-phenyl]-6-methyl-5-(4-pyridin-3-yl-pyrimidin-2-ylamino)-nicotinamide;
N-[4-(2-Dimethylamino-propyl)-phenyl]-6-methyl-5-(4-pyridin-3-yl-pyrimidin-2-ylamino)-nicotinamide;
N-[4-(2-Diethylamino-ethyl)-phenyl]-6-methyl-5-(4-pyridin-3-yl-pyrimidin-2-ylamino)-nicotinamide;
N-[4-(2-Dipropylamino-ethyl)-phenyl]-6-methyl-5-(4-pyridin-3-yl-pyrimidin-2-ylamino)-nicotinamide;
6-Methyl-5-(4-pyridin-3-yl-pyrimidin-2-ylamino)-N-(4-pyrrolidin-3-yl-phenyl)-nicotinamide;
6-Methyl-N-[4-(1-methyl-pyrrolidin-3-yl)-phenyl]-5-(4-pyridin-3-yl-pyrimidin-2-ylamino)-nicotinamide;
N-[4-(1-Ethyl-pyrrolidin-3-yl)-phenyl]-6-methyl-5-(4-pyridin-3-yl-pyrimidin-2-ylamino)-nicotinamide;
6-Methyl-N-[4-(1-propyl-pyrrolidin-3-yl)-phenyl]-5-(4-pyridin-3-yl-pyrimidin-2-ylamino)-nicotinamide;
N-[4-(1-Isopropyl-pyrrolidin-3-yl)-phenyl]-6-methyl-5-(4-pyridin-3-yl-pyrimidin-2-ylamino)-nicotinamide;
N-[4-(1-Isobutyl-pyrrolidin-3-yl)-phenyl]-6-methyl-5-(4-pyridin-3-yl-pyrimidin-2-ylamino)-nicotinamide;
N-{4-[1-(2,2-Dimethyl-propyl)-pyrrolidin-3-yl]-phenyl}-6-methyl-5-(4-pyridin-3-yl-pyrimidin-2-ylamino)-nicotinamide;
N-[4-(1-tert-Butyl-pyrrolidin-3-yl)-phenyl]-6-methyl-5-(4-pyridin-3-yl-pyrimidin-2-ylamino)-nicotinamide;
6-Methyl-N-(4-piperidin-3-yl-phenyl)-5-(4-pyridin-3-yl-pyrimidin-2-ylamino)-nicotinamide;
6-Methyl-N-[4-(1-methyl-piperidin-3-yl)-phenyl]-5-(4-pyridin-3-yl-pyrimidin-2-ylamino)-nicotinamide;
6-Methyl-N-[4-(1-methyl-piperidin-3-yl)-2-trifluoromethyl-phenyl]-5-(4-pyridin-3-yl-pyrimidin-2-ylamino)-nicotinamide;
N-[4-(1-Ethyl-piperidin-3-yl)-phenyl]-6-methyl-5-(4-pyridin-3-yl-pyrimidin-2-ylamino)-nicotinamide;
6-Methyl-N-[4-(1-propyl-piperidin-3-yl)-phenyl]-5-(4-pyridin-3-yl-pyrimidin-2-ylamino)-nicotinamide;
6-Methyl-N-(4-piperidin-4-yl-phenyl)-5-(4-pyridin-3-yl-pyrimidin-2-ylamino)-nicotinamide;
6-Methyl-N-[4-(1-methyl-piperidin-4-yl)-phenyl]-5-(4-pyridin-3-yl-pyrimidin-2-ylamino)-nicotinamide;
N-[4-(1-Ethyl-piperidin-4-yl)-phenyl]-6-methyl-5-(4-pyridin-3-yl-pyrimidin-2-ylamino)-nicotinamide;
6-Methyl-N-[4-(1-propyl-piperidin-4-yl)-phenyl]-5-(4-pyridin-3-yl-pyrimidin-2-ylamino)-nicotinamide;
6-Methyl-N-[4-(1-methyl-piperidin-4-yl)-2-trifluoromethyl-phenyl]-5-(4-pyridin-3-yl-pyrimidin-2-ylamino)-nicotinamide;
6-Methyl-N-[4-(4-methyl-piperazin-1-yl)-2-trifluoromethyl-phenyl]-5-(4-pyridin-3-yl-pyrimidin-2-ylamino)-nicotinamide;
N-[4-(1-Ethyl-piperidin-3-yl)-2-trifluoromethyl-phenyl]-6-methyl-5-(4-pyridin-3-yl-pyrimidin-2-ylamino)-nicotinamide;
6-Methyl-N-[4-(1-propyl-piperidin-3-yl)-2-trifluoromethyl-phenyl]-5-(4-pyridin-3-yl-pyrimidin-2-ylamino)-nicotinamide;
N-[4-(1-Ethyl-piperidin-4-yl)-2-trifluoromethyl-phenyl]-6-methyl-5-(4-pyridin-3-yl-pyrimidin-2-ylamino)-nicotinamide;
6-Methyl-N-[4-(1-propyl-piperidin-4-yl)-2-trifluoromethyl-phenyl]-5-(4-pyridin-3-yl-pyrimidin-2-ylamino)-nicotinamide;
N-[4-(4-Ethyl-piperazin-1-yl)-2-trifluoromethyl-phenyl]-6-methyl-5-(4-pyridin-3-yl-pyrimidin-2-ylamino)-nicotinamide;
6-Methyl-N-[4-(4-propyl-piperazin-1-yl)-2-trifluoromethyl-phenyl]-5-(4-pyridin-3-yl-pyrimidin-2-ylamino)-nicotinamide;
6-Methyl-N-(4-piperazin-1-yl-phenyl)-5-(4-pyridin-3-yl-pyrimidin-2-ylamino)-nicotinamide;
6-Methyl-N-[4-(4-methyl-piperazin-1-yl)-phenyl]-5-(4-pyridin-3-yl-pyrimidin-2-ylamino)-nicotinamide;
N-[4-(4-Ethyl-piperazin-1-yl)-phenyl]-6-methyl-5-(4-pyridin-3-yl-pyrimidin-2-ylamino)-nicotinamide;
6-Methyl-N-[4-(4-propyl-piperazin-1-yl)-phenyl]-5-(4-pyridin-3-yl-pyrimidin-2-ylamino)-nicotinamide;
6-Methyl-N-[3-(1-methyl-piperidin-3-yl)-5-trifluoromethyl-phenyl]-5-(4-pyridin-3-yl-pyrimidin-2-ylamino)-nicotinamide;
6-Methyl-N-[3-(1-methyl-piperidin-4-yl)-5-trifluoromethyl-phenyl]-5-(4-pyridin-3-yl-pyrimidin-2-ylamino)-nicotinamide;
6-Methyl-N-[3-(4-methyl-piperazin-1-yl)-5-trifluoromethyl-phenyl]-5-(4-pyridin-3-yl-pyrimidin-2-ylamino)-nicotinamide;
N-[3-(4-Ethyl-piperazin-1-yl)-5-trifluoromethyl-phenyl]-6-methyl-5-(4-pyridin-3-yl-pyrimidin-2-ylamino)-nicotinamide;
6-Methyl-N-[3-(4-propyl-piperazin-1-yl)-5-trifluoromethyl-phenyl]-5-(4-pyridin-3-yl-pyrimidin-2-ylamino)-nicotinamide;
6-Methyl-N-[3-(1-propyl-piperidin-4-yl)-5-trifluoromethyl-phenyl]-5-(4-pyridin-3-yl-pyrimidin-2-ylamino)-nicotinamide;
N-[3-(1-Ethyl-piperidin-4-yl)-5-trifluoromethyl-phenyl]-6-methyl-5-(4-pyridin-3-yl-pyrimidin-2-ylamino)-nicotinamide;
N-[3-(1-Ethyl-piperidin-3-yl)-5-trifluoromethyl-phenyl]-6-methyl-5-(4-pyridin-3-yl-pyrimidin-2-ylamino)-nicotinamide;
6-Methyl-N-[3-(1-propyl-piperidin-3-yl)-5-trifluoromethyl-phenyl]-5-(4-pyridin-3-yl-pyrimidin-2-ylamino)-nicotinamide; and a pharmaceutically acceptable salt, solvate, hydrate, cocrystal, or prodrug thereof.

10-13. (canceled)

14. A method of treating or preventing neurodenerative diseases including Parkinson's Disease, Alzheimer's Disease, amyotrophic lateral sclerosis and Huntington's disease, comprising administering to a subject in need thereof a pharmaceutically effective amount of flail the compound of claim 1.

15. The method of claim 14, wherein the subject is a human.

16. A method of treating a cancer, comprising administering to a subject in need thereof a pharmaceutically effective amount of flail the compound of claim 1.

17. The method of claim 16, wherein the subject is a human.

18. The method of claim 16, wherein the cancer is selected from the group consisting of bladder cancer, head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, kidney cancer, liver cancer, esophageal cancer, testicular cancer, gynecological cancer, thyroid cancer, Kaposi's sarcoma, viral-induced cancer, glioblastoma, glioblastoma multiforme, non-small-cell lung cancer, hepatocellular carcinoma, metastatic colon cancer, multiple myeloma, small-cell lung cancer, melanoma, and combinations thereof.
